(19) United States
(12) Patent Application Publication
(10) Pub. No.: US 2006/0088522 A1
(43) Pub. Date: Apr. 27, 2006

(54) Humanized anti-5T4 antibodies and anti-5T4/calicheamicin conjugates

(75) Inventors: Erwin R. Boghaert, Monroe, NY (US); Nitin K. Damle, Upper Saddle River, NJ (US); Davinder S. Gill, Burlington, MA (US); Kimberly A. Marquette, Somerville, MA (US); Lioudmila Tchistiakova, Andover, MA (US); Philip Ross Hamann, Thiells, NY (US); Arthur Kunz, New City, NY (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/221,902

(22) Filed: Sep. 9, 2005

Related U.S. Application Data
(60) Provisional application No. 60/608,494, filed on Sep. 10, 2004.

(51) Int. Cl. A61K 39/395; C07K 16/46; C07K 16/30

(52) U.S. Cl. 424/133.1; 424/178.1; 530/391.1; 530/387.3

(57) ABSTRACT

Chimeric and humanized anti-5T4 antibodies and antibody/drug conjugates and methods for preparing and using the same.

### RELATED APPLICATIONS

Priority is claimed to U.S. Provisional Patent Application No. 60/608,494, filed on Sep. 10, 2004, and incorporated by reference in its entirety herein.

### FIELD OF THE INVENTION

The present invention generally relates to humanized antibodies and antibody/drug conjugates (i.e., immunoconjugates) for the treatment of malignant disorders. More particularly, the present invention relates to humanized anti-5T4 antibodies, isolated variable region nucleic acids and polypeptides for preparing the antibodies, and anti-5T4/cytotoxin conjugates, particularly, anti-5T4/calicheamicin conjugates.

### BACKGROUND OF THE INVENTION

Drug conjugates developed for systemic pharmacotherapy are target-specific therapeutic agents. The concept involves coupling a therapeutic agent to a carrier molecule with binding specificity for a defined target cell population. The availability of high affinity monoclonal antibodies has fostered the development of immunotherapies, i.e., antibody-targeted drugs. Therapeutic agents that have been conjugated to monoclonal antibodies include cytotoxins, biological response modifiers, enzymes (e.g., ribonucleases), apoptosis-inducing proteins and peptides, and radioisotopes. Antibody/cytotoxin conjugates are frequently termed immunocytotoxins, whereas antibody/drug conjugates consisting of antibodies and low-molecular-weight drugs such as methothrexate and adriamycin are called chemoantibody/drug conjugates. Immunomodulators contain biological response modifiers that are known to have regulatory functions such as lymphokines, growth factors, and complement-activating cobra venom factor (CVF). Radioantibody/drug conjugates consist of radioactive isotopes, which may be used as therapeutics to kill cells by their radiation or used for imaging. Antibody-mediated drug delivery to tumor cells augments tumor-killing efficacy of the drug by minimizing its uptake in normal tissues. See e.g., Reff et al. (2002) Cancer Control 9:152-66; Sievers (2000) Cancer Chemother. Pharmacol. 46 Suppl:S1 8-22; Goldenberg (2001) Crit. Rev. Oncol. Hematol. 39:195-201. MYLOTARG® (gemtuzumab ozogamicin) is a commercially available antibody/drug conjugate that works according to this principle and which is approved for the treatment of acute myeloid leukemia in elderly patients. See Sievers et al. (1999) Blood 93: 3678-84. In this case, the targeting molecule is an anti-CD33 monoclonal antibody that is conjugated to calicheamicin.

Immunotherapy in humans has been limited, in part due to adverse responses to non-human monoclonal antibodies. Early clinical trials using rodent antibodies revealed human anti-mouse antibody (HAMA) and human anti-rat antibody (HARA) responses, which lead to rapid clearance of the antibody. Less immunogenic antibodies have since been developed, including chimeric antibodies, humanized antibodies, PRIMATIZED® antibodies, and human antibodies prepared using transgenic mice or phage display libraries. See Morrison et al. (1984) Proc. Natl. Acad. Sci. USA 81:6851-5; Queen et al. (1989) Proc. Natl. Acad. Sci. USA 86:10029-33; Newman et al. (1992) Biotechnology (NY) 10:1455-60; Green et al. (1994) Nat. Genet. 7:13-21; Marks et al. (1991) J. Mol. Biol. 222:581-97. Avoidance of a HAMA response permits high dose and repeated dose administration to achieve a therapeutic response.

Chimeric antibodies are prepared using recombinant cloning techniques to include variable regions, which contain the antigen-binding sites, from a non-human species antibody (i.e., a species immunized with the antigen) and constant regions from a human immunoglobulin. Humanized antibodies are a type of chimeric antibody, wherein only those residues of the variable regions that are responsible for antigen binding are derived from a non-human species, while the remaining variable region residues as well as the constant regions are human. Humanized antibodies are even less immunogenic than traditional chimeric antibodies and show improved stability following administration to humans. See Benincosa et al. (2000) J. Pharmacol. Exp. Ther. 292:810-6; Kalofonos et al. (1994) Eur. J. Cancer 30A:1842-50; Subramanian et al. (1998) Pediatr. Infect. Dis. J. 17:110-5.

Candidate antibodies for drug targeting include antibodies that recognize oncofetal antigens, i.e., antigens present on fetal cells and neoplastic cells, and which are largely absent from normal adult cells. See e.g., Magdelenat (1992) J. Immunol. Methods 150: 133-43. The 5T4 oncofetal antigen is a 72 kDa highly glycosylated transmembrane glycoprotein comprising a 42 kDa non-glycosylated core (Hole et al. (1988) Br. J. Cancer 57: 239-46, Hole et al. (1990) Int. J. Cancer 45: 179-84; PCT International Publication No. WO89/07947; U.S. Pat. No. 5,869,053). 5T4 includes an extracellular domain characterized by two leucine-rich repeats (LRRs) and an intervening hydrophilic region, which is an accessible antigen for targeted therapy (Myers et al. (1994) J. Biol. Chem. 269: 9319-24).

Human 5T4 is expressed in numerous cancer types, including carcinomas of the bladder, breast, cervix, endometrium, lung, esophagus, ovary, pancreas, stomach, and testes, and is substantially absent from normal tissues, except for syncytiotrophoblast in placenta (see, e.g., Southall et al. (1990) Br. J. Cancer 61: 89-95 (immunohistological distribution of 5T4 antigen in normal and malignant tissues); Mieke et al. (1997) Clin. Cancer Res. 3: 1923-1930 (low intercellular adhesion molecule 1 and high 5T4 expression on tumor cells correlate with reduced disease-free survival in colorectal carcinoma patients); Starzynska et al. (1994) Br. J. Cancer 69: 899-902 (prognostic significance of 5T4 oncofetal antigen expression in colorectal carcinoma); Starzynska et al. (1992) Br. J. Cancer 66: 867-869 (expression of 5T4 antigen in colorectal and gastric carcinoma); Jones et al. (1990) Br. J. Cancer 61: 96-100 (expression of 5T4 antigen in cervical cancer); Connor and Stern (199) Int. J. Cancer 46: 1029-1034 (loss of MHC class-I expression in cervical carcinomas); Ali et al. (2001) Oral Oncology 37: 57-64 (pattern of expression of the 5T4 oncofoetal antigen on normal, dysplastic and malignant oral mucosa); PCT International Publication No. WO89/07947; U.S. Pat. No. 5,869,053). For example, tissues reported to have no expression of 5T4 include the liver, skin, spleen, thymus, central nervous system (CNS), adrenal gland, and ovary. Tissues reported to have focal or low expression of 5T4 include the liver, skin, spleen, lymph node, tonsil, thyroid, prostate, and seminal vesicles. Weak-moderate diffuse expression of 5T4 has been reported in the kidney, lung, pancreas, pharynx, and gastrointestinal tract. The only tissue reported to have high expression of 5T4 is syncytiotrophoblast; 5T4 was also absent from normal serum or the serum of pregnant women (i.e., levels<10 ng/ml). Overexpression of 5T4 in tumors has been correlated with disease progression, and assessment of 5T4 expression has been suggested as a useful approach for identifying patients with short-term prognosis (Mulder et al. (1997) Clin. Cancer Res. 3: 1923-30, Naganuma et al. (2002) Anticancer Res. 22: 1033-8, Starzynska et al. (1994) Br. J. Cancer 69: 899-902, Starzynska et al. (1998) Eur. J. Gastroenterol. Hepatol. 10: 479-84, Wrigley et al. (1995) Int. J. Gynecol. Cancer 5: 269-274).

Several non-human anti-5T4 antibodies have been described, including mAb5T4, also called the H8 antibody, which recognizes a conformational epitope of the 5T4 antigen (Shaw et al. (2002) Biochem. J. 363: 137-45, PCT International Publication No. WO98/55607), a rat monoclonal antibody (Woods et al. (2002) Biochem. J. 366: 353-65), and the 5T4 mouse monoclonal antibody (U.S. Pat. No. 5,869,053). Single chain anti-5T4 antibodies have also been described, as well as fusion proteins that include anti-5T4 antibody sequences fused to a therapeutic molecule. For example, anti-5T4 antibody sequences fused to the human IgG1 constant domain or the extracellular domain of murine B7.1 induces cytolysis of 5T4-expressing tumor cell lines (Myers et al. (2002) Cancer Gene Ther. 9: 884-96, Shaw et al. (2000) Biochim. Biophys. Acta. 1524: 238-46; U.S. patent application Publication No. 2003/0018004). Similarly, a single chain anti-5T4 antibody fused to a superantigen may stimulate. T cell-dependent cytolysis of non-small cell lung carcinoma cells in vitro (Forsberg et al. (2001) Br. J. Cancer 85: 129-36). A phase I clinical trial using PNU-214936, a murine Fab fragment of the monoclonal antibody 5T4 fused to a mutated superantigen staphylococcal enterocytotoxin A (SEA), showed limited cytotoxicity and some anti-tumor response (Cheng et al. (2004) J. Clin. Oncol. 22(4):602-609). As an alternate therapeutic approach, recombinant 5T4 vaccines are suggested for the treatment of cancers (Mulryan et al. (2002) Mol. Cancer Ther. 1: 1129-37; UK Patent Application Publication Nos. 2,370,571 and 2,378,704; EP Patent Application Publication Nos. EP 1,160,323 and 1,152,060).

Notwithstanding substantial interest in 5T4 as a potential target for immunotherapy, therapies that employ an anti-5T4 antibody conjugated to a therapeutic agent have not been described. The present invention provides humanized anti-5T4 antibodies and antibody/drug conjugates, as well as methods for producing the disclosed antibodies and antibody/drug conjugates and methods for their therapeutic use(s).

### SUMMARY OF THE INVENTION

The present invention provides chimeric and humanized anti-5T4 antibodies and antibody fragments, and methods for preparing and using the same. The anti-5T4 antibodies of the invention comprise at least one light chain or at least one heavy chain, or fragments thereof, wherein the chimeric or humanized anti-5T4 antibody or antibody fragment (a) specifically binds to human 5T4 antigen with a binding affinity of at least about 1×10⁻⁷ M to about 1×10⁻¹² M; (b) specifically binds to human 5T4 antigen with a binding affinity greater than 1×10⁻¹¹ M; (c) specifically binds to human 5T4 antigen with a binding affinity greater than 5×10⁻¹¹ M; (d) specifically binds to human 5T4 antigen with a binding affinity greater than a binding affinity of murine H8 anti-5T4 antibody binding to human 5T4 antigen; (e) specifically targets 5T4-expressing cells in vivo; (f) competes for binding to human 5T4 antigen with an antibody of any one of (a)-(e); (g) specifically binds to an epitope bound by any one of (a)-(e); or (h) comprises an antigen binding domain of any one of (a)-(e). Chimeric and humanized anti-5T4 antibodies of the invention comprise constant regions that are derived from human constant regions, such as IgG1 or IgG4 constant regions. For example, the human IgG1 heavy chain constant region can comprise an amino acid sequence of any one of SEQ ID NOs:25 or 85-89. As another example, the human IgG4 heavy chain constant region can comprise proline at position 241.

Representative chimeric anti-5T4 antibodies of the invention include antibodies comprising (a) a light chain variable region sequence comprising amino acids 1-107 of SEQ ID NO:1, (b) heavy chain variable region sequence comprises amino acids 1-120 of SEQ ID NO:2, or (c) a light chain comprising a variable region comprising an amino acid sequence of residues 1-107 of SEQ ID NO:1, and a heavy chain comprises a variable region comprising an amino acid sequence of residues 1-120 of SEQ ID NO:2. Additional representative chimeric anti-5T4 antibodies include antibodies comprising (a) a light chain comprising an amino acid sequence of SEQ ID NO:1, and a heavy chain comprising an amino acid sequence of SEQ ID NO:2; or (b) a light chain comprising an amino acid sequence of SEQ ID NO:3, and a heavy chain comprising an amino acid sequence of SEQ ID NO:4.

Representative humanized anti-5T4 antibodies of the invention include antibodies comprising (a) framework regions comprising residues of a human antibody framework region; and (b) one or more CDRs of the light chain variable region of SEQ ID NO:17 or one or more CDRs of the heavy chain variable region of SEQ ID NO:18. For example, residues of a human antibody framework region can comprise (a) a human antibody light chain framework region of a DPK24 subgroup IV germ line clone, a VκIII subgroup, or a VκI subgroup germ line clone; (b)a human antibody heavy chain framework region selected from the group consisting of DP-75, DP-8(VH1-2), DP-25, VI-2b and VI-3 (VH1-03), DP-15 and V1-8 (VH1-08), DP-14 and V1-18 (VH1-18), DP-5 and V1-24P (VH1-24), DP-4 (VH1-45), DP-7 (VH1-46), DP-10, DA-6 and YAC-7 (VH1-69), DP-88 (VH1-e), DP-3 and DA-8 (VH1-f); (c) a consensus sequence of a heavy chain framework region of (b); or (d) a framework region that is at least 95% identical to a framework region of (a)-(c).

Representative humanized anti-5T4 antibodies of the invention can also include two or more CDRs of SEQ ID NOs:17 or 18, such as two or all three CDRs of the light chain variable region of SEQ ID NO:17, or two or all three CDRs of the heavy chain variable region of SEQ ID NO:18, or one or more CDRs or the light chain variable region of SEQ ID NO:17 and one or more CDRs of the heavy chain variable region of SEQ ID NO:18, or all of the CDRs or SEQ ID NOs:17 and 18.

Representative humanized anti-5T4 antibodies of the invention can also comprise a light chain variable region comprising (a) an amino acid sequence of SEQ ID NO:17 or 23; (b) an amino acid sequence that is at least 78% identical to SEQ ID NO:17; or (c) an amino acid sequence that is at least 81% identical to SEQ ID NO:23. Similarly, humanized anti-5T4 antibodies of the invention can comprise a light chain variable region sequence encoded by a nucleic acid comprising: (a) a nucleotide sequence of SEQ ID NO:22 or 81; (b) a nucleotide sequence that is at least 90% identical to the nucleic acid of SEQ ID NO:22; (c)a nucleotide sequence that is at least 91% identical to the nucleic acid of SEQ ID NO:81; or (d) a nucleic acid that specifically hybridizes to the complement of SEQ ID NO:22 or SEQ ID NO:81 under stringent hybridization conditions.

Representative humanized anti-5T4 antibodies of the invention can also comprise a heavy chain variable region comprising (a) an amino acid sequence set forth as any one of SEQ ID NOs:18, 19, and 21; (b) an amino acid sequence that is at least 83% identical to SEQ ID NO:18; (c) an amino acid sequence that is at least 81% identical to SEQ ID NO:19; or (d) an amino acid sequence that is at least 86% identical to SEQ ID NO:21. Similarly, humanized antibodies of the invention can comprise a heavy chain variable region sequence encoded by a nucleic acid comprising (a) a nucleotide sequence of SEQ ID NO:20, 82, or 83; (b) a nucleotide sequence that is at least 91% identical to the nucleic acid of SEQ ID NO:20 or SEQ ID NO:83; (c) a nucleotide sequence that is at least 94% identical to the nucleic acid of SEQ ID NO:82; or (d) a nucleic acid that specifically hybridizes to the complement of any one of SEQ ID NOs:20, 82, and 83 under stringent hybridization conditions.

Additional representative humanized anti-5T4 antibodies of the invention include antibodies comprising (a) a light chain variable region comprising an amino acid sequence of residues 1-107 of SEQ ID NO:5, and a heavy chain variable region comprising an amino acid sequence of residues 1-120 of SEQ ID NO:6; (b) a light chain amino acid sequence of SEQ ID NO:5, and a heavy chain amino acid sequence of SEQ ID NO:6; (c) a light chain variable region comprising an amino acid sequence of residues 1-107 of SEQ ID NO:7, and a heavy chain variable region comprising an amino acid sequence of residues 1-120 of SEQ ID NO:8; (d) a light chain amino acid sequence of SEQ ID NO:7, and a heavy chain amino acid sequence of SEQ ID NO:8; (e) a light chain variable region comprising an amino acid sequence of residues 1-107 of SEQ ID NO:9, and a heavy chain variable region comprising an amino acid sequence of residues 1-120 of SEQ ID NO:10; (f) a light chain amino acid sequence of SEQ ID NO:9, and a heavy chain amino acid sequence of SEQ ID NO:10; (g) a light chain variable region comprising an amino acid sequence of residues 1-107 of SEQ ID NO:11, and a heavy chain variable region comprising an amino acid sequence of residues 1-120 of SEQ ID NO:12; (h) a light chain amino acid sequence of SEQ ID NO:11, and a heavy chain amino acid sequence of SEQ ID NO:12; (i) a light chain variable region comprising an amino acid sequence of residues 1-107 of SEQ ID NO:11, and a heavy chain variable region comprising an amino acid sequence of SEQ ID NO:19; (j) a light chain amino acid sequence of SEQ ID NO:11, and a heavy chain amino acid sequence of SEQ ID NO:84; (k) a light chain variable region comprising an amino acid sequence of residues 1-107 of SEQ ID NO:11, and a heavy chain variable region comprising an amino acid sequence of residues 1-120 of SEQ ID NO:8; and (l) a light chain amino acid sequence of SEQ ID NO:11, and a heavy chain amino acid sequence of SEQ ID NO:8.

Also provided are antibody/drug conjugates for drug delivery comprising (a) a chimeric or humanized anti-5T4 antibody or antibody fragment of the invention; and (b) a drug, which is directly or indirectly bound to the antibody. Representative drugs include therapeutic agents, such as cytotoxins, radioisotopes, immunomodulatory agents, anti-angiogenic agents, anti-proliferative agents, pro-apoptotic agents, chemotherapeutic agents, and therapeutic nucleic acids. A cytotoxin may be, for example, an antibiotic, an inhibitor of tubulin polymerization, an alkylating agent, a protein synthesis inhibitor, a protein kinase inhibitor, a phosphatase inhibitor, a topoisomerase inhibitor, or an enzyme. Antibiotic cytotoxins, such as calicheamicin, calicheamicin, N-acetyl-γ-calicheamicin, or derivatives thereof such as N-acetyl-γ-calicheamicin dimethyl hydrazide, are particularly useful for anti-cancer therapies.

The disclosed anti-5T4 antibody/drug conjugates may include a linker for binding the antibody to the drug. Representative linkers include 4-(4′acetylphenoxy)butanoic acid (AcBut), 3-acetylphenyl acidic acid (AcPac), and 4-mercapto-4-methyl-pentanoic acid (Amide). The antibody/drug conjugates may also include polyethylene glycol or other agents to enhance drug incorporation.

The present invention further provides a method for preparing antibody/drug conjugates having the formula:
5T4Ab(—X—W)ₘ
wherein 5T4Ab is a chimeric or humanized anti-5T4 antibody or antibody fragment; X is a linker that comprises a product of any reactive group that may react with an anti-5T4 antibody; W is a drug; m is the average loading for a purified conjugation product; and (—X—W)ₘ is a drug derivative. According to the method, the drug derivative is added to the chimeric or humanized anti-5T4 antibody or antibody fragment wherein the drug is 3-10% by weight of the chimeric or humanized anti-5T4 antibody or antibody fragment. The drug derivative and the chimeric or humanized anti-5T4 antibody or antibody fragment are then incubated in a non-nucleophilic, protein-compatible, buffered solution having a pH in a range from about 7 to 9 to produce an antibody/drug conjugate, wherein the solution further comprises (i) a suitable organic cosolvent, and (ii) one or more additives comprising at least one bile acid or its salt, and wherein the incubation is conducted at a temperature ranging from about 30° C. to about 35° C. for a period of time ranging from about 15 minutes to about 24 hours. The resultant conjugate is then subjected to a chromatographic separation process to separate antibody/drug conjugates with a loading in the range of 3-10% by weight drug and with low conjugated fraction (LCF) from unconjugated chimeric or humanized anti-5T4 antibody or antibody fragment, drug derivative, and aggregated conjugates. Antibody/drug conjugates produced by the method are also provided.

For delivery of a drug to 5T4-expressing cells, the present invention provides methods whereby cells are contacted with an antibody/drug conjugate comprising (i) a chimeric or humanized anti-5T4 antibody, and (ii) a drug which is bound to the humanized anti-5T4 antibody directly or indirectly. According to the disclosed methods, the drug is internalized within the target cell. Therapeutic methods are also disclosed herein, which comprise administering to the subject having a 5T4-positive cancer a therapeutically effective amount of an anti-5T4 antibody/drug conjugate comprising (i) a chimeric or humanized anti-5T4 antibody or antibody fragment, and (ii) a therapeutic agent which is bound to the humanized anti-5T4 antibody or antibody fragment directly or indirectly. Anti-5T4 therapies of the invention may be combined with any other known therapy for improved effect. A second therapeutic agent may be administered in combination with an anti-5T4 antibody/drug conjugate simultaneously or consecutively in any order.

### BRIEF DESCRIPTION OF THE DRAWINGS

FIG. 1 shows the results of Western blot analysis to assess 5T4 expression in tumorigenic cell lines. Western blots were generated from lysates of cultured cells as well as from allografted tumors in nude mice. CT26/neo, CT26 mouse colon carcinoma cells expressing the neomycin resistance gene; CT26/5T4, CT26 cells expressing 5T4 antigen.

FIG. 2 shows the results of Western blot analysis of CT26/5T4 and CT26/neo samples after exposure of the cell lines to biotin. Sample A is the fraction of 5T4 that has been biotinylated and capable of binding to avidin. Sample S is the residual amount of 5T4 present in the supernatant after precipitation of the cell extract with avidin. This represents a fraction that has not been biotinylated and is therefore located in the cell plasma. 5T4 is detected in both membrane (A) and intracellular (S) fractions.

FIGS. 3A-3B show the results of experiments to quantify intracellular versus membrane-associated 5T4 antigen. FIG. 3A shows Western blots prepared using CT26/5T4 cell extracts diluted as indicated. The biotinylated sample represents the residual amount of 5T4 present in the sample following depletion of the biotinylated sample using avidin, i.e., the amount of non-membrane-associated 5T4. The total sample represents the sum of the residual amount and the amount depleted by avidin, i.e., amount of non-membrane-associated and membrane associated 5T4 antigen. FIG. 3B shows the linear regression curves determined by the dilution of the sample and the optical density of the H8-reactive band. The amount of membrane-associated 5T4 antigen is depicted as the difference between the optical density of the total sample and the optical density of the biotinylated sample after avidin depletion. As described in Example 1, the amount of 5T4 on the cell membrane (5T4M) was calculated to be 24% of total cellular 5T4 in CT26/5T4 cells.

FIG. 4 shows Western blot results that demonstrate 5T4 antigen on the cell surface of CT26/5T4 cells, DLD-1 cells (human colon carcinoma cells), N87 cells (human gastric carcinoma cells), PC3-MM2 cells (human prostate carcinoma cells), and PC3 cells (human prostate carcinoma cells).

FIGS. 5A-5B show results of FACS analysis to detect membrane localization of 5T4 antigen. In MDAMB435/neo cells, the signal of H8 coincides with that of a control IgG (FIG. 5A). In contrast, in MDAMB435/5T4 cells, the signal resulting from the H8 antibody is more than 100-fold greater than that of the control antibody, indicating the presence of 5T4 on the cell membrane (FIG. 5B). Black, detection of 5T4 antigen; gray, detection by a control IgG.

FIG. 6 show results of FACS analysis to detect 5T4 antigen on the membranes of N87 (human gastric carcinoma cells), PC14PE6 (human lung carcinoma cells), and NCl-H157 cells (human lung carcinoma cells). In each case, the signal resulting from the H8 antibody is about 10-fold greater than that of the control antibody, indicating the presence of 5T4 on the cell membrane. Gray, detection of 5T4 antigen; black, detection by a control IgG.

FIG. 7 is a line graph depicting measurements of fluorescently labeled H8 antibody detected on the cell surface of CT26/5T4 cells and in the cell culture medium. The mean fluorescence of membrane-associated antibody decreased as a function of time. The antibody was not released in the medium. These results demonstrate that the H8 antibody/5T4 complex is internalized by CT26/5T4 cells.

FIG. 8 is a line graph depicting selective cytolysis of MDAMB435/5T4 cells exposed to an anti-5T4 conjugate comprising H8 antibody conjugated to calicheamicin using 4-mercapto-4-methyl-pentanoic acid as a linker.

FIGS. 9A-9B are line graphs that depict selective cytolysis of 5T4-expressing cells exposed to an anti-5T4 conjugate (H8PEG2K-AcBut-CalichDMH) comprising PEGylated H8 antibody conjugated to calicheamicin using 4-(4′-acetylphenoxy)butanoic acid (AcBut) as a linker. See Example 2. FIG. 9A shows that MDAMB435/neo cells lacking 5T4 antigen are approximately equally susceptible to cytolysis by H8PEG2K-AcBut-CalichDMH as by free calicheamicin. FIG. 9B shows enhanced cytolysis of 5T4-expressing cells exposed to H8PEG2K-AcBut-CalichDMH as compared to free calicheamicin.

FIG. 10 is a line graph that depicts growth inhibition of MDAMB435/5T4 tumors exposed to H8-calicheamicin conjugates prepared using the indicated linkers. PBS, phosphate buffered saline; H8-AcBut-CalichDMH, H8 antibody conjugated to calicheamicin using 4-(4′-acetylphenoxy)butanoic acid (AcBut); H8-AcPac-CalichDMH, H8 antibody conjugated to calicheamicin using 3-acetylphenyl acidic acid; H8-Amide-CalichDMH, H8 antibody conjugated to calicheamicin using 4-mercapto-4-methyl-pentanoic acid; H8PEG(mal2)-AcBut-CalichDMH, PEGylated H8 antibody conjugated to calicheamicin using 4-(4′-acetylphenoxy)butanoic acid (AcBut).

FIGS. 11A-11B are line graphs that depict growth inhibition of MDAMB435/5T4 tumors in the presence of control substances (FIG. 11A) or H8-calicheamicin conjugates (FIG. 11B). CMA, anti-CD33 antibody conjugated to calicheamicin (negative control, i.e., used to assess cytotoxicity due to tumor uptake of a conjugate by cells lacking the targeted antigen); PBS, phosphate buffered saline; H8+CalichDMH, a mixture of H8 antibody and calicheamicin (unconjugated); CalichDMH, free calicheamicin; H8-AcPac-CalichDMH, H8 antibody conjugated to calicheamicin using 3-acetylphenyl acidic acid; H8-amide-CalichDMA, H8 antibody conjugated to calicheamicin using 4-mercapto-4-methyl-pentanoic acid.

FIG. 12 is a line graph that depicts growth inhibition of NCl-H157 tumors exposed to the indicated H8-calicheamicin conjugates or control substances. H8-AcPac-CalichDMH, H8 antibody conjugated to calicheamicin using 3-acetylphenyl acidic acid; H8-amide-CalichDMA, H8 antibody conjugated to calicheamicin using 4-mercapto-4-methyl-pentanoic acid; CMA, anti-CD33 antibody conjugated to calicheamicin (negative control); PBS, phosphate buffered saline; H8, unconjugated H8 antibody.

FIGS. 13A-13B are line graphs that depict growth inhibition of N87 tumors in the presence of control substances (FIG. 13A) or H8-calicheamicin conjugates (FIG. 13B). CMA, anti-CD33 antibody conjugated to calicheamicin (positive control); PBS, phosphate buffered saline; H8+CalichDMH, a mixture of H8 antibody and calicheamicin (unconjugated); CalichDMH, free calicheamicin; H8-AcPac-CalichDMH, H8 antibody conjugated to calicheamicin using 3-acetylphenyl acidic acid; H8-amide-CalichDMA, H8 antibody conjugated to calicheamicin using 4-mercapto-4-methyl-pentanoic acid.

FIG. 14 is a line graph that depicts growth inhibition of PC14PE6 tumors exposed to H8/calicheamicin conjugates or control substances. H8-AcPac-CalichDMH, H8 antibody conjugated to calicheamicin using 3-acetylphenyl acidic acid; H8-amide-CalichDMA, H8 antibody conjugated to calicheamicin using 4-mercapto-4-methyl-pentanoic acid; CMA, anti-CD33 antibody conjugated to calicheamicin (negative control); PBS, phosphate buffered saline; H8, unconjugated H8 antibody.

FIGS. 15A-15G are images of normal and tumor-infested lungs of an orthotopic model of lung cancer. FIG. 15A is a picture of an excised normal mouse lung; the heart appears dark. FIG. 15B is a picture of an excised mouse lung infested with tumor nodules following intravenous injection of PC14PE6 tumor cells (see Example 4); H, heart. FIG. 15C is a macroscopic image (4× magnification) showing the thorax after collapse of the lungs. Lung nodules (LN) are distinguishable from normal lung tissue (L). The thoracic cavity was filled with hemorrhagic fluid (pleural effusion, PE). FIGS. 15D-15G are photomicrographs of hematoxylin and eosin stained sections of paraffin-embedded lung and heart tissue, which demonstrate the extent of tumor infiltration and destruction of normal tissue. FIGS. 15D-15E show infiltrates of tumor cells in the pleural cavity (15D) and the pericardium (15E). FIGS. 15F-15G show the reduction of functional lung tissue by proliferating tumor tissue in the perialveolar space.

FIG. 16 is a line graph showing the surviving fraction (%) of mice bearing orthotopic lung tumors that have received the indicated treatments. All treatments were administered intraperitoneally 6 days after injection of the PC14PE6 cells. See Example 4. H8 (thick solid black line), unconjugated murine H8 antibody; PBS (solid white line), phosphate-buffered saline; CMA 2 (thin solid black line), anti-CD33 antibody conjugated to calicheamicin administered at a dose of 2 μg calicheamicin; CMA 4 (line with small dashes), anti-CD33 antibody conjugated to calicheamicin administered at a dose of 4 μg calicheamicin; H8-AcPac-CalichDMH 2 (line with large dashes), H8-calicheamicin conjugate administered at a dose of 2 μg calicheamicin; H8-AcPac-CalichDMH 4 (line with large dashes), H8-calicheamicin conjugate administered at a dose of 4 μg calicheamicin. The results for H8-calicheacmicin conjugate administered at a dose of 2 μg or at a dose of 4 μg were indistinguishable over a period of 120 days. Ten animals were included in each treatment group. Each treatment regimen consisted of 3 doses administered intraperitoneally with 4 days interval between each dose.

FIG. 17 is a bar graph showing pleural volumes in mice that died from lung tumors following the indicated control treatments. PBS, phosphate buffered saline; H8, unconjugated H8 antibody; CMA 2, f anti-CD33 antibody conjugated to calicheamicin administered at 2 μg per dose; CMA 4, anti-CD33 antibody conjugated to calicheamicin administered at 4 μg per dose; n, number of animals. Pleural effusion volume was not reduced following administration of unconjugated H8 antibody or the control conjugate CMS.

FIG. 18 is an alignment of the murine H8 light chain variable region (amino acids 21-127 of SEQ ID NO:16) and the DPK24 germ line clone (SEQ ID NO:63). Boxed sequences, CDRs; asterisks, positions at which amino acids of murine H8 are maintained in humanized H8 light chain variable region version 1, and at which amino acids of human DPK24 are maintained in humanized light chain variable region version 2; underlined residues, mutations that increase antibody expression.

FIG. 19 is an alignment of human light chain variable region sequences of subgroup VκIII (SEQ ID NOs:65-70) and the murine H8 light chain variable region (amino acids 21-127 of SEQ ID NO:16). Residues that differ in human framework sequences when compared to H8 framework sequences are underlined. For humanization of H8, one or more residues at the corresponding positions in H8 is substituted with a residue of a human framework sequence. Boxed sequences, CDRs.

FIG. 20 is an alignment of human light chain variable region sequences of subgroup VκI (SEQ ID NOs:71-80) and the murine H8 light chain variable region (amino acids 21-127 of SEQ ID NO:16). For humanization of H8, one or more residues at the corresponding positions in H8 is substituted with a residue of a human framework sequence. Boxed sequences, CDRs.

FIG. 21 is an alignment of the murine H8 heavy chain variable region (amino acids 20-139 of SEQ ID NO:14) and the DP75 germ line clone (SEQ ID NO:64). Boxed sequences, CDRs; asterisks, positions at which amino acids of murine H8 are maintained in humanized H8 heavy chain variable region version 1 (i.e., K38, S40, and I48), and at which amino acids of human DP75 are maintained in humanized heavy chain variable region version 2.

FIG. 22 is an alignment of human heavy chain variable region sequences of subgroup I (SEQ ID NOs:52-60) and the consensus framework sequences derived there from (SEQ ID NO:49-51).

FIG. 23 is an alignment of the murine H8 heavy chain variable region (amino acids 20-139 of SEQ ID NO:14) and the humanized H8 heavy chain variable region derived from the consensus sequence of heavy chain variable region subgroup I, i.e., humanized heavy chain variable region version 3 (SEQ ID NO:19). Boxed sequences, CDRs.

FIGS. 24A-24C show sequences of representative light chain variable region sequences (FIG. 24A) and heavy chain variable region sequences (FIGS. 24B-24C) of humanized anti-5T4 antibodies.

FIGS. 25A-25O show the results of BLAST analysis performed using humanized variable regions as query sequences. See also Tables 6 and 7.

FIGS. 26A-26B shows the sequences of representative human constant regions used to prepare humanized anti-5T4 antibodies.

FIGS. 27A-27G show the light chain and heavy chain amino acid sequences of representative anti-5T4 antibodies. FIG. 27A shows a chimeric anti-5T4 antibody having (a) a light chain comprising the murine H8 light chain variable region and a human kappa constant region (SEQ ID NO:1), and (b) a heavy chain comprising the murine H8 heavy chain variable region and a human IgG1 constant region (SEQ ID NO:2). FIG. 27B shows a chimeric anti-5T4 antibody having (a) a light chain comprising the murine H8 light chain variable region and a human kappa constant region (SEQ ID NO:3), and (b) a heavy chain comprising the murine H8 heavy chain variable region and a mutated human IgG4 constant region (SEQ ID NO:4). FIG. 27C shows a semi-human anti-5T4 antibody having (a) a light chain comprising the humanized H8 light chain variable region version 1 and a human kappa constant region (SEQ ID NO:5), and (b) a heavy chain comprising the murine H8 heavy chain variable region and a mutated human IgG4 constant region (SEQ ID NO:6). FIG. 27D shows a humanized anti-5T4 antibody having (a) a light chain comprising the humanized H8 light chain variable region version 1 and a human kappa constant region (SEQ ID NO:7), and (b) a heavy chain comprising the humanized H8 heavy chain variable region version 1 and a mutated human IgG4 constant region (SEQ ID NO:8). FIG. 27E shows a humanized anti-5T4 antibody having (a) a light chain comprising the humanized H8 light chain variable region version 1 and a human kappa constant region (SEQ ID NO:9), and (b) a heavy chain comprising the humanized H8 heavy chain variable region version 2 and a human IgG1 constant region (SEQ ID NO:10). FIG. 27F shows a humanized anti-5T4 antibody having (a) a light chain comprising the humanized H8 light chain variable region version 2 and a human kappa constant region (SEQ ID NO:11), and (b) a heavy chain comprising the humanized H8 heavy chain variable region version 2 and a mutated human IgG4 constant region (SEQ ID NO:12). FIG. 27G shows a humanized anti-5T4 antibody having (a) a light chain comprising the humanized H8 light chain variable region version 2 and a human kappa constant region (SEQ ID NO:11), and (b) a heavy chain comprising the humanized H8 heavy chain variable region version 3 and a mutated human IgG4 constant region (SEQ ID NO:84). Single underlining, variable regions; boxed sequences, CDRs; asterisk, proline mutation.

FIGS. 28A-28B show results of FACS analysis to detect 5T4 antigen on MDAMB435/neo cells (FIG. 28A) or on MDAMB435/5T4 cells (FIG. 28B) using murine H8, chimeric versions of H8, and humanized versions of H8 at the indicated concentrations. All antibodies show selective binding to MDAMB435/5T4 cells.

FIG. 29 is a line graph that shows the binding properties of chimeric H8 antibody and humanized H8 versions 1-3, which were determined using a competitive binding assay. The IC50 for the chimeric H8 antibody and humanized H8 versions 1-3 were 1.0×10⁻⁹M, 1.0×10⁻⁹M, 1.4×10⁻⁹M, and 1.5×10⁻⁹M, respectively. See Example 5.

FIG. 30 is a line graph that shows detection of chimeric H8 antibody and humanized H8 antibody on the cell surface of MDAMB435/5T4 cells over a period of 25 hours. The reduced level of detection over the observation period demonstrates internalization of both antibodies. No detectable antibody was present in the conditioned medium during the course of the experiment.

FIG. 31 is a bar graph depicting levels of transient expression of chimeric H8 antibody and humanized H8 versions 1-3 in COS-1 cells. The three humanized H8 antibodies were expressed at a similar level (version 1, 4.4 mg/L/48 hours; version 2, 2.7 mg/L/48 hours; version 3, 3.9 mg/L/48 hours) which was greater than that observed for chimeric H8 antibody (0.6 mg/L/48 hours). See Example 6.

FIGS. 32A-32B are line graphs that show inhibition of spheroid growth of MDAMB435/neo and MDAMB435/5T4 cells in vitro following 144 hours of exposure to H8-AcBut-CalichDMH, (humanized H8 antibody conjugated to calicheamicin using 4-(4′-acetylphenoxy)butanoic acid (AcBut)) at the indicated concentrations.

FIGS. 33A-33C are line graphs that depict growth inhibition of N87 tumors in the presence of control substances (FIG. 33A) or humanized H8-calicheamicin conjugates (FIG. 33B) and response calculations (FIG. 33C). PBS, phosphate buffered saline; huH8+CalichDMH, a mixture of H8 antibody and calicheamicin (unconjugated); CMA, anti-CD33 antibody conjugated to calicheamicin; CMC, anti-CD22 antibody conjugated to calicheamicin; huH8-AcBut-CalichDMH, humanized H8 antibody conjugated to calicheamicin using 4-(4′-acetylphenoxy)butanoic acid (AcBut); (4), antibody-calicheamicin conjugate administered at a dose of 4 μg calicheamicin; (2), antibody-calicheamicin conjugate administered at a dose of 2 μg calicheamicin; (1), antibody-calicheamicin conjugate administered at a dose of 1 μg calicheamicin; arrows, dosing schedule on days 1, 5, and 9; CR, complete response; PR, partial response; TR, no response; NR, no response. See Example 9.

FIGS. 34A-34C are line graphs that depict growth inhibition of MDAMB435/5T4 tumors in the presence of control substances (FIG. 34A) or humanized H8-calicheamicin conjugates (FIG. 34B) and response calculations (FIG. 34C). PBS, phosphate buffered saline; huH8+CalichDMH, a mixture of H8 antibody and calicheamicin (unconjugated); CMA, anti-CD33 antibody conjugated to calicheamicin; CMC, anti-CD22 antibody conjugated to calicheamicin; huH8-AcBut-CalichDMH, humanized H8 antibody conjugated to calicheamicin using 4-(4′-acetylphenoxy)butanoic acid (AcBut); (4), antibody-calicheamicin conjugate administered at a dose of 4 μg calicheamicin; (2), antibody-calicheamicin conjugate administered at a dose of 2 μg calicheamicin; (1), antibody-calicheamicin conjugate administered at a dose of 1 μg calicheamicin arrows, dosing schedule on days 1, 5, and 9; CR, complete response; PR, partial response; TR, no response; NR, no response. See Example 9.

FIGS. 35A-35E are line graphs that depict growth inhibition of PC14PE6 tumors in the presence of control substances (FIG. 35A) or humanized H8-calicheamicin conjugates (FIGS. 35B, 35D, 35E) and calculated responses (FIG. 35C). FIGS. 35A-35C present data pertaining to new growth tumors, and FIG. 35D presents data pertaining to treatment of relapsed tumors. PBS, phosphate buffered saline; huH8+CalichDMH, a mixture of H8 antibody and calicheamicin (unconjugated); CMA, anti-CD33 antibody conjugated to calicheamicin; huH8-AcBut-CalichDMH, humanized H8 antibody conjugated to calicheamicin using 4-(4′-acetylphenoxy)butanoic acid (AcBut); (4), antibody-calicheamicin conjugate administered at a dose of 4 μg calicheamicin; (2), antibody-calicheamicin conjugate administered at a dose of 2 μg calicheamicin; (1), antibody-calicheamicin conjugate administered at a dose of 1 μg calicheamicin; (4*), antibody-calicheamicin conjugate administered at a dose of 4 μg calicheamicin after tumors allowed to grow to approximately 1.08 cm³ prior to treatment with the conjugate; arrows, dosing schedule on days 1, 5, and 9 (FIGS. 35A, 35B, and 35D) or days 19, 23, and 27 (FIG. 35C); CR, complete response; PR, partial response; TR, no response; NR, no response. See Example 9.

FIGS. 36A-36B show photographs of mice harboring PC14PE6 tumors 21 days following treatment with vehicle (phosphate buffered saline) (FIG. 367A) or with huH8-AcBut-CalichDMH (humanized H8 antibody conjugated to calicheamicin using 4-(4′-acetylphenoxy)butanoic acid (AcBut)) (FIG. 36B). PC14PE6 tumors were approximately 80 mm³ at the time vehicle or humanized H8-calicheamicin conjugate were administered. Agents were administered by intraperitoneal injection in a total of three doses of 4 μg calicheamicin per dose, each dose separated by three days. Arrow in FIG. 36A identifies visible tumor. Area circumscribed by dotted line in FIG. 36B identifies area where PC14PE6 tumor has regressed. See Example 9.

### DETAILED DESCRIPTION OF THE INVENTION

I. Chimeric and Humanized Anti-5T4 Antibodies

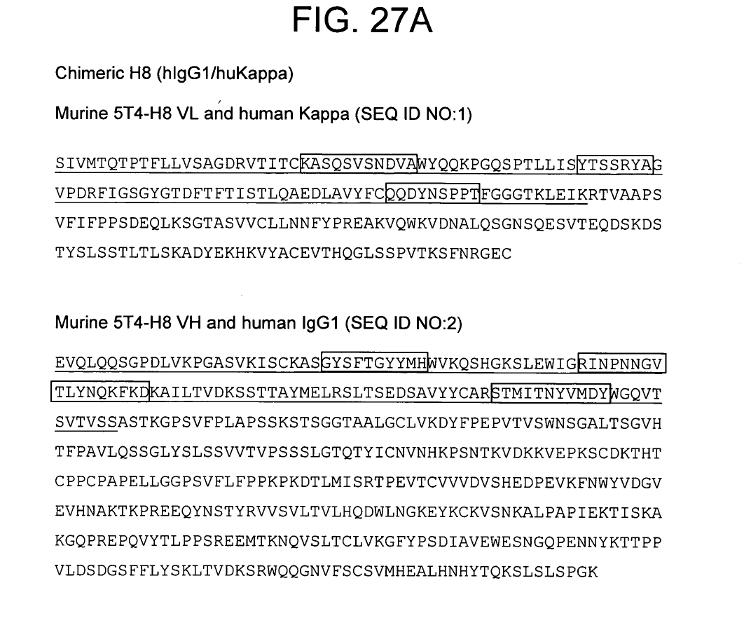

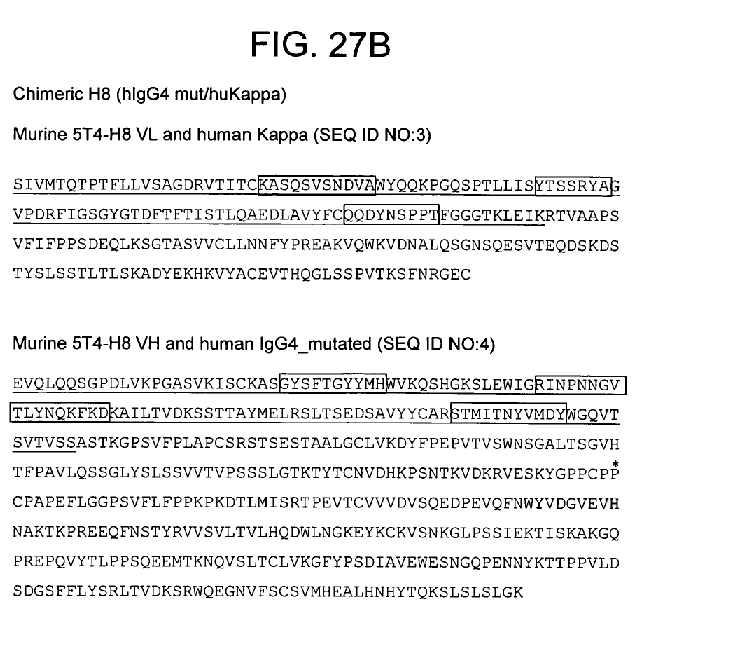

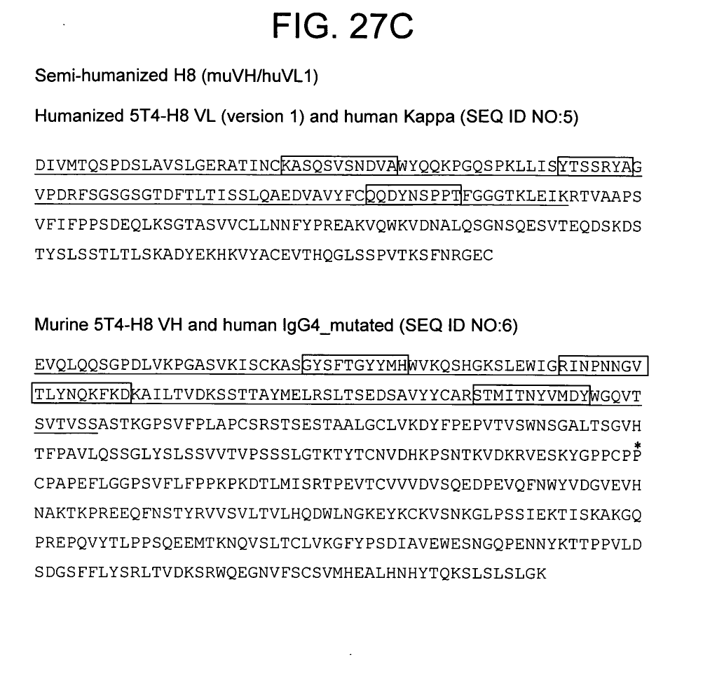

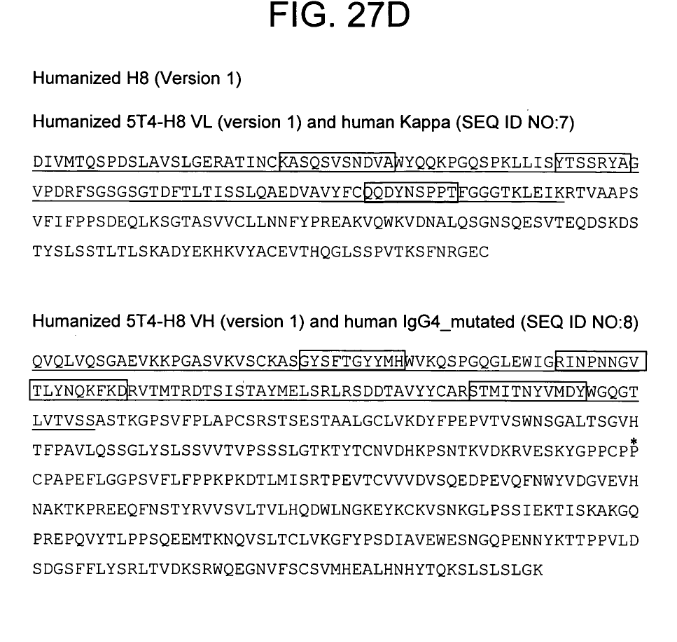

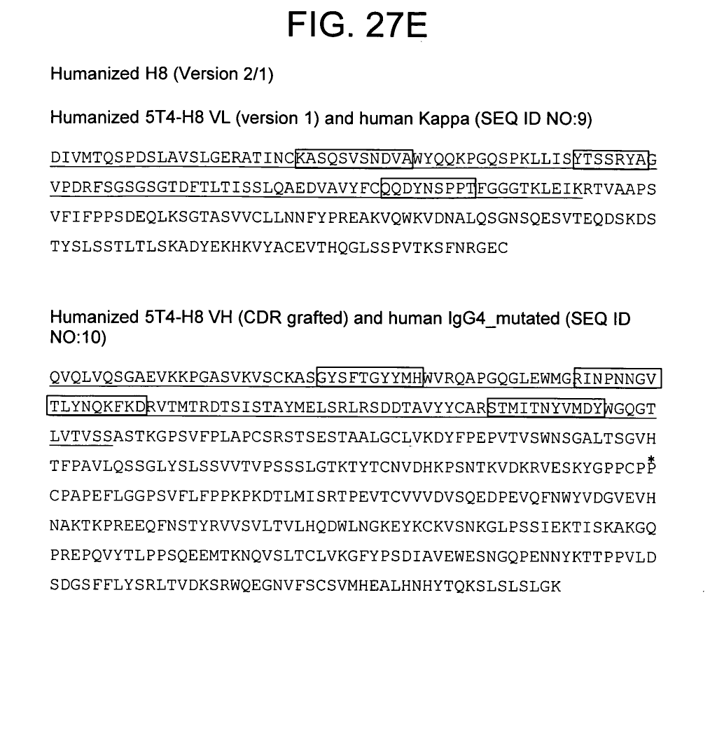

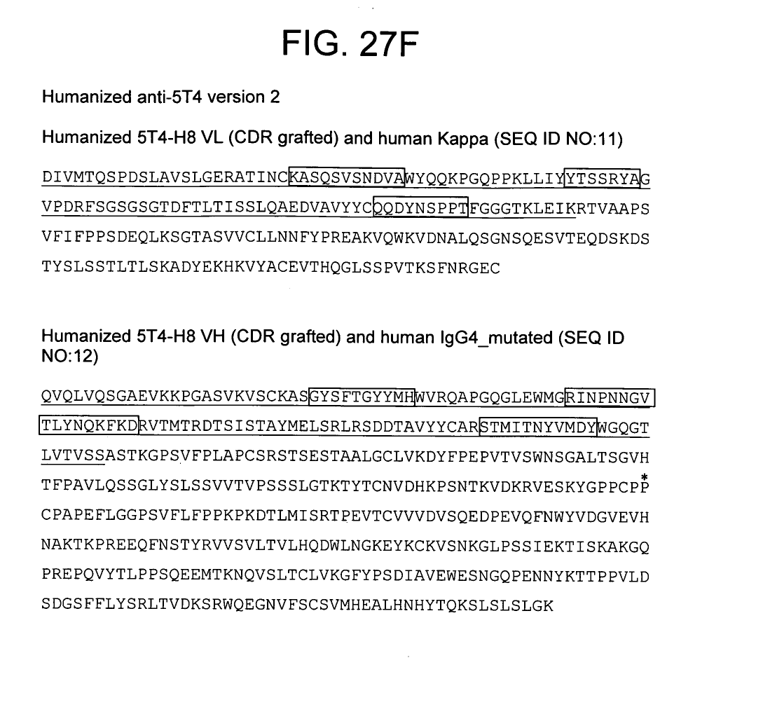

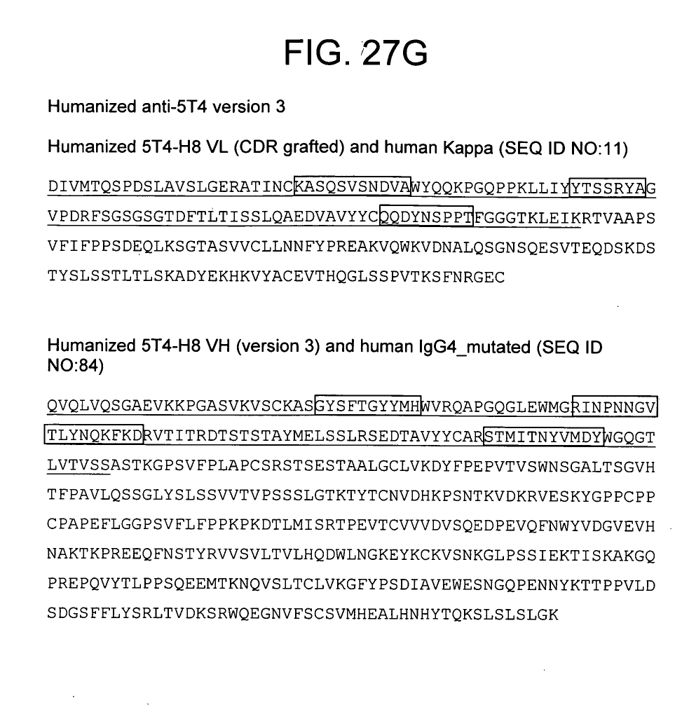

H8 is a hybridoma-generated monoclonal mouse IgG1 antibody which is described in PCT International Publication No. WO 98/55607 and in Forsberg et al. (1997) J. Biol. Chem. 272(19):124430-12436. Chimeric anti-5T4 antibodies of the invention include variable region sequences of the murine anti-5T4 antibody and additional residues derived from human antibody sequences. Humanized anti-5T4 antibodies of the invention include antigen binding residues from mouse anti-5T4 antibody H8 and additional residues derived from human antibody sequences. The disclosed chimeric and humanized anti-5T4 antibodies are therefore also called chimeric H8 antibodies and humanized H8 antibodies. Representative chimeric and humanized H8 antibodies are set forth in FIGS. 27A-27F.

The term antibody refers to an immunoglobulin protein, or antibody fragments that comprise an antigen binding site (e.g., Fab, modified Fab, Fab′, F(ab′)₂ or Fv fragments, or a protein having at least one immunoglobulin light chain variable region or at least one immunoglobulin heavy chain region). Humanized antibodies of the invention include diabodies, tetrameric antibodies, single chain antibodies, tretravalent antibodies, multispecific antibodies (e.g., bispecific antibodies), domain-specific antibodies that recognize a particular epitope (e.g., antibodies that recognize an epitope bound by the H8 antibody).

The term anti-5T4 antibody refers to an antibody that specifically binds to 5T4 antigen, particularly human 5T4 antigen. The 5T4 antigen is a 72 kDa non-glycosylated phosphoprotein found on the surface of trophoblast cells and numerous cancer cell types See Hole et al. (1988) Br. J. Cancer 57: 239-46, Hole et al. (1990) Int. J. Cancer 45: 179-184; PCT International Publication No. WO89/07947; U.S. Pat. No. 5,869,053.

The term binding refers to an affinity between two molecules, for example, an antigen and an antibody. As used herein, specific binding means a preferential binding of an antibody to an antigen in a heterogeneous sample comprising multiple different antigens. The binding of an antibody to an antigen is specific if the binding affinity is at least about 10⁻⁷ M or higher, such as at least about 10⁻⁸ M or higher, including at least about 10⁻⁹ M or higher, at least about 10⁻¹¹ M or higher, or at least about 10⁻¹² M or higher. For example, specific binding of an antibody of the invention to a human 5T4 antigen includes binding in the range of at least about 1×10⁻⁷ to about 1×10⁻¹². Specific binding of an antibody of the invention to a human 5T4 antigen also includes binding in the range of at least about 3×10⁻¹⁰ M to about 12×10⁻¹⁰ M, such as within the range of about 4×10⁻¹⁰ M to about 9×10⁻¹⁰ M, or such as within the range of about 7×10⁻¹⁰ M to about 12×10⁻¹⁰ M, or such as within the range of about 7×10⁻¹⁰ M to about 9×10⁻¹⁰ M, or such as within the range of about 9×10⁻¹⁰ M to about 12×10⁻¹⁰ M, or such as within the range of about 11×10⁻¹⁰ M to about 12×10⁻¹⁰ M, or greater binding affinities such as about 1.0×10⁻¹¹ M to about 10×10⁻¹¹ M, or about 1.0×10⁻¹¹ M to about 5×10⁻¹¹ M, or about 5.0×10⁻¹¹ M to about 10×10⁻¹¹ M. The phrase specifically binds also refers to selective targeting to 5T4-expressing cells when administered to a subject.

The term chimeric antibody is used herein to describe an antibody comprising sequences from at least two different species. Humanized antibodies are one type of chimeric antibody. A chimeric anti-5T4 antibody may comprise (a) a light chain variable region having an amino acid sequence of residues 1-107 of SEQ ID NO:1 and a heavy chain variable region having an amino acid sequence of residues 1-120 of SEQ ID NO:2; (b) a light chain amino acid sequence of SEQ ID NO:1 and a heavy chain amino acid sequence of SEQ ID NO:2; or (c) a light chain amino acid sequence of SEQ ID NO:3 and a heavy chain amino acid sequence of SEQ ID NO:4.

The term humanized is used herein to describe an antibody, wherein variable region residues responsible for antigen binding (i.e., residues of a complementarity determining region and any other residues that participate in antigen binding) are derived from a non-human species, while the remaining variable region residues (i.e., residues of the framework regions) and constant regions are derived, at least in part, from human antibody sequences. Residues of the variable regions and variable regions and constant regions of a humanized antibody may also be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species.

Representative chimeric and humanized anti-5T4 antibodies of the invention comprise at least one light chain or at least one heavy chain, or fragments thereof, wherein the chimeric or humanized anti-5T4 antibody or antibody fragment (a) specifically binds to human 5T4 antigen with a binding affinity of at least about 1×10⁻⁷ M to about 1×10⁻¹² M; (b) specifically binds to human 5T4 antigen with a binding affinity greater than 1×10⁻¹¹ M; (c) specifically binds to human 5T4 antigen with a binding affinity greater than 5×10⁻¹¹ M; (d) specifically binds to human 5T4 antigen with a binding affinity greater than a binding affinity of murine H8 anti-5T4 antibody binding to human 5T4 antigen; (e) specifically targets 5T4-expressing cells in vivo; (f) competes for binding to human 5T4 antigen with an antibody of any one of (a)-(e); (g) specifically binds to an epitope bound by any one of (a)-(e); or (h) comprises an antigen binding domain of any one of (a)-(e).

The murine H8 anti-5T4 antibody has been shown to recognize a conformational epitope proximal to transmembrane domain of 5T4. Glycosylation, which is important for structure and immunogenicity, and intramolecular disulphide bonds are required for binding of the antibody. It has also been shown that the H8 anti-5T4 antibody does not bind mouse 5T4, although there is 84% identity between mouse and human 5T4 and 6 of 7 N-linked glycosylation sites are conserved between the two. The N-terminal and C-terminal cysteines are also completely conserved between mouse and human 5T4. The murine H8 antibody has also been shown to bind human 5T4 when the N-linked glycosylation site at amino acid 192 is removed (Shaw et al. (2002) Biochem J. 365: 137-145). There is some evidence suggesting that the H8 anti-5T4 antibody does not bind a human/mouse 5T4 chimera having mouse LRR2 (residues 173-361 replacing human residues 173-355) and yet the antibody does bind to the reciprocal chimera. There is also evidence suggesting that both a chimeric H8 antibody and a humanized H8 antibody bind to a 5T4 chimera containing mouse residues 282-361. This evidence leads to the conclusion that the H8 epitope is located between amino acids 173 and 252. Additional evidence suggests that chimeric H8 may not bind to a human/mouse anti-5T4 chimera containing mouse residues 173-258, while a humanized H8 antibody has slight binding at higher concentrations.

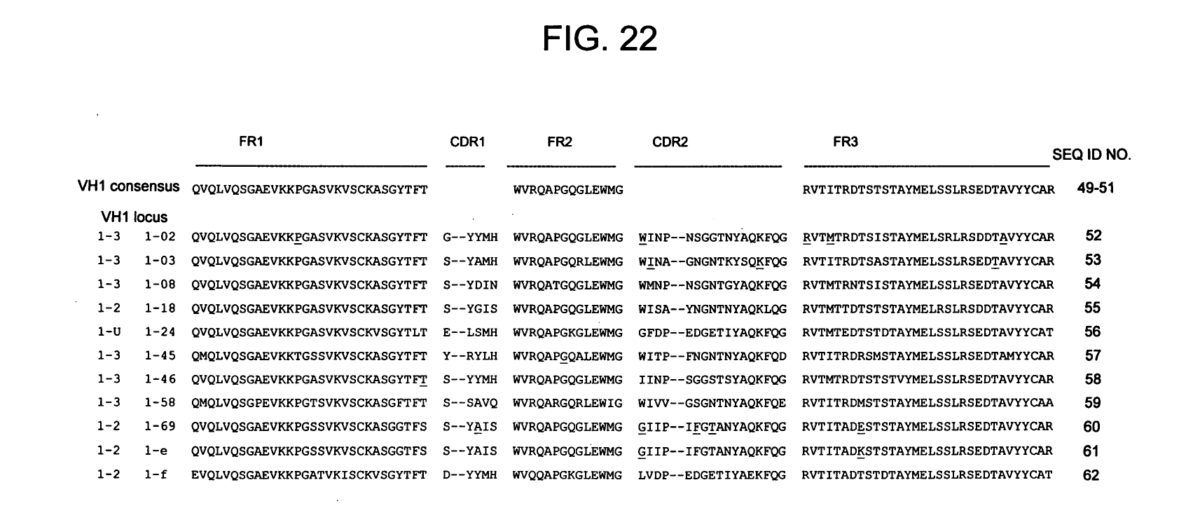

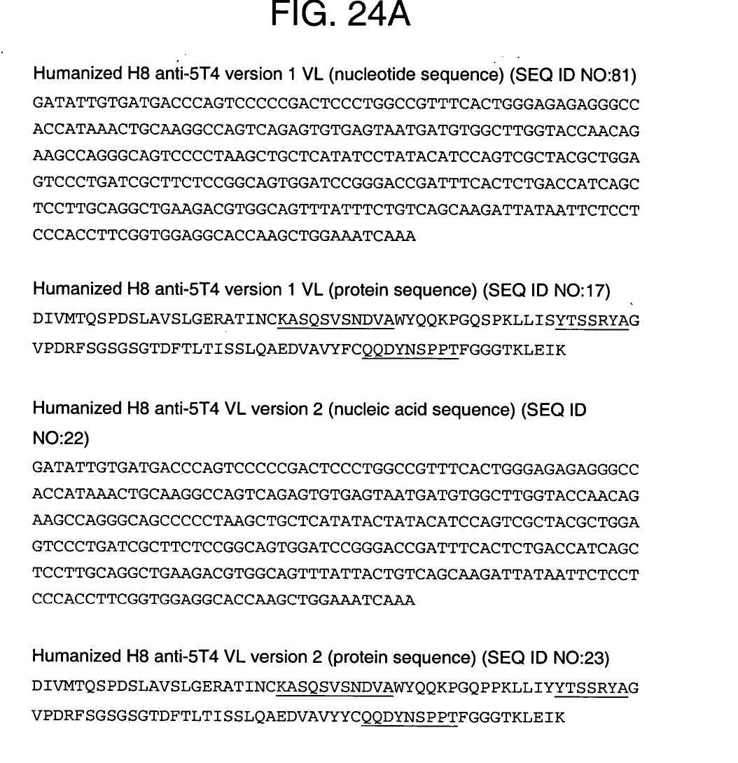

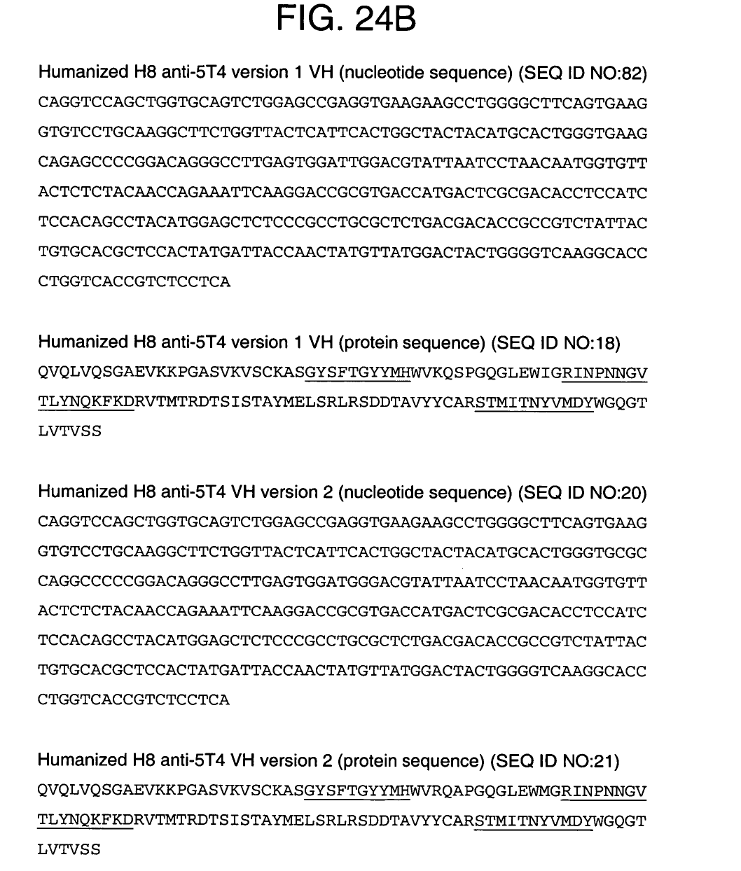

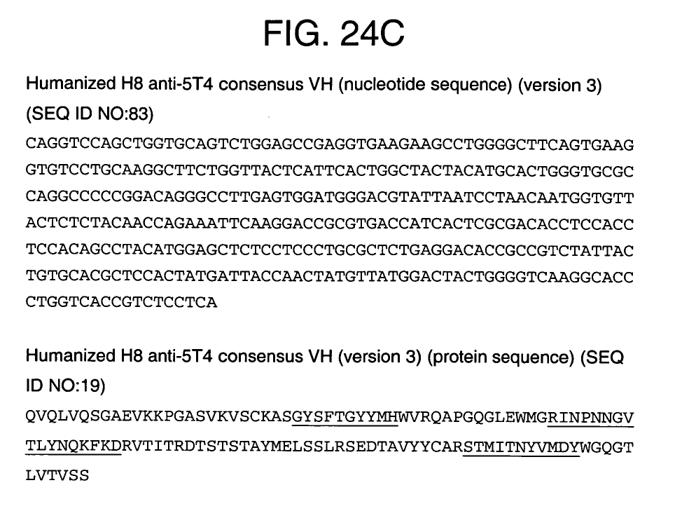

Naturally occurring antibodies are tetrameric (H₂L₂) glycoproteins of about 150,000 daltons, composed of two identical light (L) chains and two identical heavy (H) chains. The two heavy chains are linked to each other by disulfide bonds and each heavy chain is linked to a light chain by a disulfide bond. Each of the light and heavy chains is further characterized by an amino-terminal variable region and a constant region. The term variable refers to the fact that certain portions of the variable domains differ extensively in sequence among antibodies and substantially determine the binding affinity and specificity of each particular antibody for its particular antigen. The variable regions of each of light and heavy chain align to form the antigen-binding domain. Representative humanized H8 variable regions are set forth in FIGS. 24A-22C (SEQ ID NOs:17, 18, 19, 21, and 23).

Antibodies having a tetrameric structure, similar to naturally occurring antibodies, may be recombinantly prepared using standard techniques. Recombinantly produced antibodies also include single chain antibodies, wherein the variable regions of a single light chain and heavy chain pair include an antigen binding region, and fusion proteins, wherein a variable region of a humanized anti-5T4 antibody is fused to an effector sequence, such as an Fc domain, a cytokine, an immunostimulant, a cytotoxin, or any other therapeutic protein. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561.

Tetravalent antibodies (H₄L₄) comprising two intact tetrameric antibodies, including homodimers and heterodimers, may be prepared for example as described in PCT International Publication No. WO 02/096948. Antibody dimers may also be prepared via introduction of cysteine residue(s) in the antibody constant region, which promote interchain disulfide bond formation, using heterobifunctional cross-linkers (Wolff et al. (1993) Cancer Res. 53: 2560-5), or by recombinant production to include a dual constant region (Stevenson et al. (1989) Anticancer Drug Des. 3: 219-30).

The term complementarity determining region or CDR refers to residues of the antibody variable regions that participate in antigen binding. A number of definitions of the CDRs are in common use. The Kabat definition is based on sequence variability, and the Chothia definition is based on the location of the structural loop regions. The AbM definition is a compromise between the Kabat and Chothia approaches. The CDRs of the light chain variable region are bounded by the residues at positions 24 and 34 (CDR1-L), 50 and 56 (CDR2-L), and 89 and 97 (CDR3-L) according to the Kabat, Chothia, or AbM algorithm. According to the Kabat definition, the CDRs of the heavy chain variable region are bounded by the residues at positions 31 and 35B (CDR1-H), 50 and 65 (CDR2-H), and 95 and 102 (CDR3-H) (numbering according to Kabat). According to the Chothia definition, the CDRs of the heavy chain variable region are bounded by the residues at positions 26 and 32 (CDR1-H), 52 and 56 (CDR2-H), and 95 and 102 (CDR3-H) (numbering according to Chothia). According to the AbM definition, the CDRs of the heavy chain variable region are bounded by the residues at positions 26 and 35B (CDR1-H), 50 and 58 (CDR2-H), and 95 and 102 (CDR3-H) (numbering according to Kabat). See Martin et al. (1989) Proc. Natl. Acad. Sci. USA 86: 9268-9272; Martin et al. (1991) Methods Enzymol. 203: 121-153; Pedersen et al. (1992) Immunomethods 1: 126; and Rees et al. (1996) In Sternberg M. J. E. (ed.), Protein Structure Prediction, Oxford University Press, Oxford, pp.141-172.

The term specificity determining region or SDR refers to those residues within CDRs that directly interact with antigen, which correspond to hypervariable residues. See (Padlan et al. (1995) FASEB J. 9: 133-9).

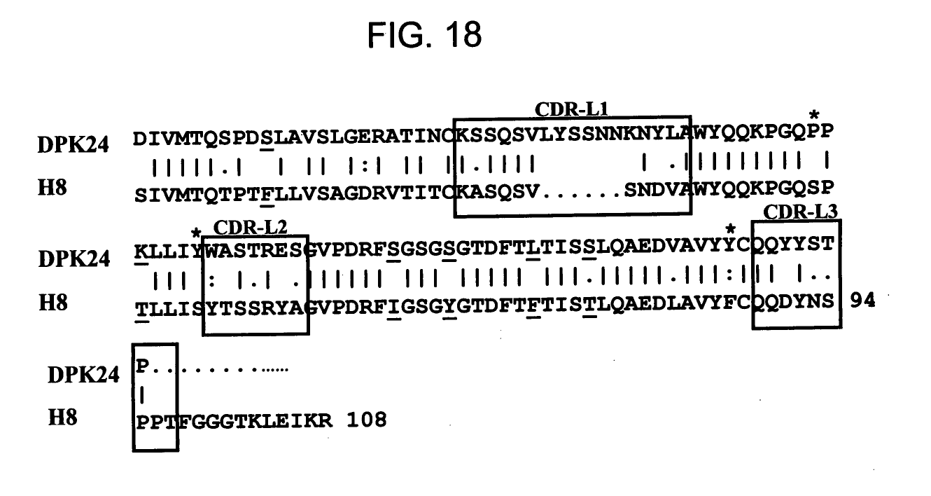

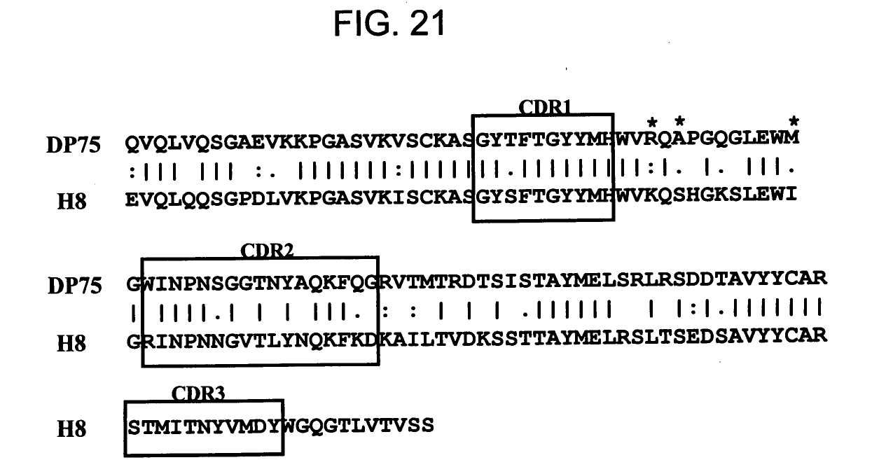

Framework residues are those residues of the variable region other than hypervariable residues. Representative human frameworks of a heavy chain variable region that may be used to prepare humanized anti-5T4 antibodies include the framework regions of DP-75 and DP-8(VH1-2), DP-25, VI-2b and VI-3 (VH1-03), DP-15 and V1-8 (VH1-08), DP-14 and V1-18 (VH1-18), DP-5 and V1-24P (VH1-24), DP-4 (VH1-45), DP-7 (VH1-46), DP-10, DA-6 and YAC-7 (VH1-69), DP-88 (VH1-e), DP-3, and DA-8 (VH1-f). Consensus framework sequences based on the foregoing individual sequences may also be used. See FIGS. 21-23. Representative human frameworks of a light chain variable region include that of human germ line clone DPK24 and germ line clone subgroups VκIII and VκI, each of which shows greater than 60% amino acid identity when compared to the H8 light chain variable region. See FIGS. 18-20.

The constant regions of the disclosed humanized anti-5T4 antibodies are derived from constant regions from any one of IgA, IgD, IgE, IgG, IgM, and any isotypes thereof (e.g., IgG1, IgG2, IgG3, or IgG4 isotypes of IgG). The choice of the human isotype (IgG1, IgG2, IgG3, IgG4) and modification of particular amino acids in the human isotype may enhance or eliminate activation of host defense mechanisms and alter biodistribution of a humanized antibody of the invention. See (Reff et al. (2002) Cancer Control 9: 152-66).

Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. These general approaches may be combined with standard mutagenesis and synthesis techniques to produce an anti-5T4 antibody of any desired sequence.

Veneering is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells. See Padlan (1991) Mol. Immunol. 28:489-98. A non-human antibody is veneered by (1) identifying exposed exterior framework region residues in the non-human antibody, which are different from those at the same positions in framework regions of a human antibody, and (2) replacing the identified residues with amino acids that typically occupy these same positions in human antibodies.

Grafting of CDRs is performed by replacing one or more CDRs of an acceptor antibody (e.g., a human antibody) with CDRs of a donor antibody (e.g., a non-human antibody). Acceptor antibodies may be selected based on similarity of framework residues between a candidate acceptor antibody and a donor antibody and may be further modified to introduce similar residues. For example, a human acceptor framework may comprise a heavy chain variable region of a human sub-group I consensus sequence, optionally with non-human donor residues at one or more of positions 1, 28, 48, 67, 69, 71, and 93. As another example, a human acceptor framework may comprise a light chain variable region of a human sub-group I consensus sequence, optionally with non-human donor residues at one or more of positions 2, 3, 4, 37, 38, 45 and 60. Following CDR grafting, additional changes may be made in the donor and/or acceptor sequences to optimize antibody binding and functionality. See e.g., PCT International Publication No. WO 91/09967.

Grafting of abbreviated CDRs is a related approach. Abbreviated CDRs include the specificity-determining residues and adjacent amino acids, including those at positions 27d-34, 50-55 and 89-96 in the light chain, and at positions 31-35b, 50-58, and 95-101 in the heavy chain (numbering convention of (Kabat et al. (1987)). See (Padlan et al. (1995) FASEB J. 9: 133-9). Grafting of specificity-determining residues (SDRs) is premised on the understanding that the binding specificity and affinity of an antibody combining site is determined by the most highly variable residues within each of the complementarity determining regions (CDRs). Analysis of the three-dimensional structures of antibody-antigen complexes, combined with analysis of the available amino acid sequence data was used to model sequence variability based on structural dissimilarity of amino acid residues that occur at each position within the CDR. See Padlan et al. (1995) FASEB J. 9: 133-139. Minimally immunogenic polypeptide sequences consisting of contact residues, which are referred to as specificity-determining residues (SDRs), are identified and grafted onto human framework regions.

According to the Frankenstein approach, human framework regions are identified as having substantial sequence homology to each framework region of the relevant non-human antibody, and CDRs of the non-human antibody are grafted onto the composite of the different human framework regions. A related method also useful for preparation of antibodies of the invention is described in U.S. patent application Publication No. 2003/0040606.

Humanized anti-5T4 antibodies disclosed herein typically comprise at least one humanized light chain variable region or heavy chain variable region. Thus, a humanized anti-5T4 antibody of the invention may comprise a light chain variable region prepared by veneering, grafting of abbreviated CDRs or SDRs, or Frankenstein assembly, as above, and a heavy chain variable region of a non-human antibody (e.g., the H8 antibody or other non-human anti-5T4 antibody). Alternatively, a light chain variable region of a non-human antibody may be combined with a humanized heavy chain variable region.

Representative humanized anti-5T4 antibodies of the invention include (a) antibodies having one or more CDRs of a non-human anti-5T4 antibody selected from CDRs of the light chain variable region of SEQ ID NO:17 or the heavy chain variable region of SEQ ID NO:18, such as two or more CDRs selected from CDRs of the light chain variable region of SEQ ID NO:17 or the heavy chain variable region of SEQ ID NO:18; (b) antibodies having a light chain comprising a variable region having two or three CDRs of SEQ ID NO:17; and (c) antibodies having a heavy chain comprising a variable region having two or three CDRs of SEQ ID NO:18. Representative humanized anti-5T4 antibodies of the invention also include those antibodies having (a) a light chain variable region amino acid sequence set forth as SEQ ID NO:17 or 23; (b) a light chain variable region amino acid sequence that is at least 78% identical to SEQ ID NO:17; or (c) a light chain variable region amino acid sequence that is at least 81% identical to SEQ ID NO:23. A light chain variable region of a functional humanized anti-5T4 antibody (i.e., an anti-5T4 antibody that specifically binds to 5T4 antigen) may be encoded by (a) a nucleic acid of SEQ ID NO:22 or SEQ ID NO:81; (b) a nucleic acid that is at least 90% identical to the nucleic acid of SEQ ID NO:22; (c) a nucleic acid that is at least 91% identical to the nucleic acid of SEQ ID NO:81; or (d) a nucleic acid that specifically hybridizes to the complement of SEQ ID NO:22 or SEQ ID NO:81 under stringent hybridization conditions, for example final wash conditions of 0.1×SSC at 65° C.

Representative humanized anti-5T4 antibodies of the invention further include those antibodies having (a) a heavy chain variable region amino acid sequence set forth as any one of SEQ ID NOs:18, 19, and 21; (b) a heavy chain variable region amino acid sequence that is at least 83% identical to SEQ ID NO:18; (c) a heavy chain variable region amino acid sequence that is at least 81% identical to SEQ ID NO:19; or (d) a heavy chain variable region amino acid sequence that is at least 86% identical to SEQ ID NO:21. A heavy chain variable region of a functional humanized anti-5T4 antibody (i.e., an anti-5T4 antibody that specifically binds to 5T4 antigen) may be encoded by (a) a nucleic acid of any one of SEQ ID NOs:20, 82, and 83; (b) a nucleic acid that is at least 91% identical to the nucleic acid of SEQ ID NO:20; (c) a nucleic acid that is at least 94% identical to the nucleic acid of SEQ ID NO:82; (d) a nucleic acid that is at least 91% identical to the nucleic acid of SEQ ID NO:83; or (e) a nucleic acid that specifically hybridizes to the complement of any one of SEQ ID NOs:20, 82, and 83 under stringent hybridization conditions, for example final wash conditions of 0.1×SSC at 65° C.

A humanized anti-5T4 antibody may comprise (a) a light chain variable region having an amino acid sequence of residues 1-107 of SEQ ID NO:5 and a heavy chain variable region having an amino acid sequence of residues 1-120 of SEQ ID NO:6; (b) a light chain amino acid sequence of SEQ ID NO:5 and a heavy chain amino acid sequence of SEQ ID NO:6; (c) a light chain variable region having an amino acid sequence of residues 1-107 of SEQ ID NO:7 and a heavy chain variable region having an amino acid sequence of residues 1-120 of SEQ ID NO:8; (d) a light chain amino acid sequence of SEQ ID NO:7 and a heavy chain amino acid sequence of SEQ ID NO:8; (e) a light chain variable region having an amino acid sequence of residues 1-107 of SEQ ID NO:9 and a heavy chain variable region having an amino acid sequence of residues 1-120 of SEQ ID NO:10; (f) a light chain amino acid sequence of SEQ ID NO:9 and a heavy chain amino acid sequence of SEQ ID NO:10; (g) a light chain variable region having an amino acid sequence of residues 1-107 of SEQ ID NO:11 and a heavy chain variable region having an amino acid sequence of residues 1-120 of SEQ ID NO:12; or (h) a light chain amino acid sequence of SEQ ID NO:11 and a heavy chain amino acid sequence of SEQ ID NO:12.

Humanized anti-5T4 antibodies of the invention may be constructed wherein the variable region of a first chain (i.e., the light chain variable region or the heavy chain variable region) is humanized, and wherein the variable region of the second chain is not humanized (i.e., a variable region of an antibody produced in a non-human species). These antibodies are referred to herein as semi-humanized antibodies. For example, an anti-5T4 antibody may comprise a humanized light chain variable region of SEQ ID NO:17 or 23, and a heavy chain variable region of a non-human anti-5T4 antibody, such as the murine H8 heavy chain variable region of SEQ ID NO:14. Alternatively, an anti-5T4 antibody may comprise a humanized light chain variable region of a non-human anti-5T4 antibody, such as the murine H8 light chain variable region of SEQ ID NO:16, and a humanized heavy chain variable region of any one of SEQ ID NOs:18, 19, or 21. Anti-5T4 non-human antibodies other than murine H8 may be used to prepare semi-humanized antibodies, for example the rat monoclonal antibody described by Woods et al. (2002) Biochem. J. 366: 353-65).

Variants of the disclosed humanized anti-5T4 antibodies may be readily prepared to include various changes, substitutions, insertions, and deletions, where such changes provide for advantages in use. For example, to increase the serum half life of the antibody, a salvage receptor binding epitope may be incorporated, if not present already, into the antibody heavy chain sequence. See U.S. Pat. No. 5,739,277. Additional modifications to enhance antibody stability include modification of IgG4 to replace the serine at residue 241 with proline. See Angal et al. (1993) Mol. Immunol. 30: 105-108. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

Variants of humanized anti-5T4 antibodies of the invention may be produced using standard recombinant techniques, including site-directed mutagenesis, or recombination methods. A diversified repertoire of humanized anti-5T4 antibodies may be prepared via gene arrangement and gene conversion methods in transgenic non-human animals (U.S. patent Publication No. 2003/0017534), which are then tested for relevant activities using functional assays. In particular embodiments of the invention, anti-5T4 variants are obtained using an affinity maturation protocol such as mutating the CDRs (Yang et al. (1995) J. Mol. Biol. 254: 392-403), chain shuffling (Marks et al. (1992) Biotechnology (NY) 10: 779-783), use of mutator strains of E. coli (Low et al. (1996) J. Mol. Biol. 260: 359-368), DNA shuffling (Patten et al. (1997) Curr. Opin. Biotechnol. 8: 724-733), phage display (Thompson et al. (1996) J. Mol. Biol. 256: 77-88), and sexual PCR (Crameri et al. (1998) Nature 391: 288-291). For immunotherapy applications, relevant functional assays include specific binding to human 5T4 antigen, internalization of the antibody when conjugated to a cytotoxin, and targeting to a tumor site(s) when administered to a tumor-bearing animal, as described in the Examples. See Examples 1-11.

The present invention further provides cells and cell lines expressing humanized anti-5T4 antibodies of the invention. Representative host cells include mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int Rev. Immunol. 10(2-3):103-112). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

I.A. Chimeric and Humanized Anti-5T4 Nucleic Acids

The present invention further provides isolated nucleic acids encoding humanized anti-5T4 light and heavy chain variable regions. The isolated nucleic acids may be used to prepare a humanized anti-5T4 antibody, as disclosed herein.

The terms nucleic acid molecule and nucleic acid each refer to deoxyribonucleotides or ribonucleotides and polymers thereof in single-stranded, double-stranded, or triplexed form. Unless specifically limited, the term encompasses nucleic acids containing known analogues of natural nucleotides that have similar properties as the reference natural nucleic acid. The terms nucleic acid molecule or nucleic acid may also be used in place of gene, cDNA, mRNA, or cRNA. Nucleic acids may be synthesized, or may be derived from any biological source, including any organism. Representative methods for cloning nucleic acids that encode humanized anti-5T4 antibodies are described in Example 5.

Representative nucleic acids of the invention comprise the nucleotide sequence of any one of SEQ ID NOs:20, 22, 81, 82, or 83. Nucleic acids of the invention may also comprise a nucleotide sequence that is substantially identical to any one of SEQ ID NOs:20, 22, 81, 82, or 83, for example, at least 91% identical to any one of SEQ ID NOs:20, 81, or 83, or at least 90% identical to SEQ ID NO:22, or at least 94% identical to SEQ ID NO:82. Sequences are compared for maximum correspondence using a sequence comparison algorithm using the full-length sequence of any one of SEQ ID NOs:20, 22, 81, 82, or 83 as the query sequence, as described herein below, or by visual inspection. See also Example 5 and Table 6.

With respect to substantially identical nucleic acids having a specified minimal percentage identity to the disclosed humanized H8 variable region nucleic acids, the substantially identical sequences may also be at least about 92% identical to SEQ ID NO:20. 81, or 83, such as at least 93% identical, or at least 94% identical, or at least 95% identical, or at least 96% identical, or at least 97% identical, or at least 98% identical, or at least 99% identical. Similarly, substantially identical sequences also include sequences that are at least about 91% identical to SEQ ID NO:22, for example, at least about 92% identical, or at least 93% identical, or at least 94% identical, or at least 95% identical, or at least 96% identical, or at least 97% identical, or at least 98% identical, or at least 99% identical; and sequences that are at least 95% identical to SEQ ID NO:82, such as at least 96% identical, or at least 97% identical, or at least 98% identical, or at least 99% identical.

Substantially identical sequences may be polymorphic sequences. The term polymorphic refers to the occurrence of two or more genetically determined alternative sequences or alleles in a population. An allelic difference may be as small as one base pair.

Substantially identical sequences may also comprise mutagenized sequences, including sequences comprising silent mutations. A mutation may comprise one or more residue changes, a deletion of one or more residues, or an insertion of one or more additional residues.

Substantially identical nucleic acids are also identified as nucleic acids that hybridize specifically to or hybridize substantially to the full length of any one of SEQ ID NOs:20, 22, 81, 82, and 83 under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of SEQ ID NOs:20, 22, 81, 82, and 83. Such fragments may be readily prepared, for example by chemical synthesis of the fragment, by application of nucleic acid amplification technology, or by introducing selected sequences into recombinant vectors for recombinant production.

The phrase hybridizing specifically to refers to the binding, duplexing, or hybridizing of a molecule only to a particular nucleotide sequence under stringent conditions when that sequence is present in a complex nucleic acid mixture (e.g., total cellular DNA or RNA).

The phrase hybridizing substantially to refers to complementary hybridization between a probe nucleic acid molecule and a target nucleic acid molecule and embraces minor mismatches that may be accommodated by reducing the stringency of the hybridization media to achieve the desired hybridization.

Stringent hybridization conditions and stringent hybridization wash conditions in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under stringent conditions a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1 M Na⁺ ion, typically about 0.01 to 1M Na⁺ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code.

The term conservatively substituted variants refers to nucleic acid sequences having degenerate codon substitutions wherein the third position of one or more selected (or all) codons is substituted with mixed-base and/or deoxyinosine residues. See Batzer et al. (1991) Nucleic Acids Res. 19:5081; Ohtsuka et al. (1985) J. Biol. Chem. 260:2605-2608; and Rossolini et al. (1994) Mol. Cell Probes 8:91-98.

Nucleic acids of the invention also comprise nucleic acids complementary to any one of SEQ ID NOs:20, 22, 81, 82, and 83, and subsequences and elongated sequences of the nucleic acids and complementary nucleic acids of any one of SEQ ID NOs:20, 22, 81, 82, and 83.

The term complementary sequences, as used herein, indicates two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

The term subsequence refers to a sequence of nucleic acids that comprises a part of a longer nucleic acid sequence. An exemplary subsequence is a probe, described herein above, or a primer. The term primer as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term elongated sequence refers to an addition of nucleotides (or other analogous molecules) incorporated into the nucleic acid. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, intronic sequences, additional restriction enzyme sites, multiple cloning sites, and other coding segments. Thus, the invention also provides vectors comprising the disclosed nucleic acids, including vectors for recombinant expression, wherein a nucleic acid of the invention is operatively linked to a functional promoter.

The term operatively linked, as used herein, refers to a functional combination between a promoter region and a nucleotide sequence such that the transcription of the nucleotide sequence is controlled and regulated by the promoter region. Techniques for operatively linking a promoter region to a nucleotide sequence are known in the art.

The term vector is used herein to refer to a nucleic acid molecule having nucleotide sequences that enable its replication in a host cell. A vector may also include nucleotide sequences to permit ligation of nucleotide sequences within the vector, wherein such nucleotide sequences are also replicated in a host cell. Representative vectors include plasmids, cosmids, and viral vectors.

Nucleic acids of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/New York; Ausubel (ed.) (1995) Short Protocols in Molecular Biology, 3rd ed. Wiley, N.Y.

I.B. Chimeric and Humanized Anti-5T4 Polypeptides

The present invention also provides isolated humanized anti-5T4 polypeptides. Representative light chain and heavy chain polypeptides of the invention are set forth as SEQ ID NOs:1-12. Representative light chain variable region polypeptide and heavy chain variable region polypeptides are set forth as SEQ ID NOs:17 and 23 and SEQ ID NOs:18, 19, and 21, respectively.

The terms polypeptide and protein each refer to a compound made up of a single chain of amino acids joined by peptide bonds. The antibodies of the invention are alternately referred to as polypeptides or proteins. Polypeptides of the invention may comprise naturally occurring amino acids, synthetic amino acids, genetically encoded amino acids, non-genetically encoded amino acids, and combinations thereof. Polypeptides may include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

The present invention also provides functional fragments of a humanized anti-5T4 polypeptide, for example, a variable region polypeptide. Functional polypeptide sequences that are longer than the disclosed sequences are also provided. For example, one or more amino acids may be added to the N-terminus or C-terminus of an antibody polypeptide. Such additional amino acids may be employed in a variety of applications, including but not limited to purification applications. Methods of preparing elongated proteins are known in the art.

Polypeptides of the invention include (a) a light chain variable region polypeptide having the amino acid sequence of SEQ ID NO:17 or 23; (b) a light chain variable region polypeptide having an amino acid sequence that is at least 78% identical to SEQ ID NO:17; and (c) a light chain variable region polypeptide having an amino acid sequence at least 81% identical to SEQ ID NO:23. Additional polypeptides of the invention include (a) a heavy chain variable region polypeptide having the amino acid sequence set forth as any one of SEQ ID NOs:18, 19, and 21; (b) a heavy chain variable region polypeptide having an amino acid sequence that is at least 83% identical to SEQ ID NO:18; (c) a heavy chain variable region polypeptide having an amino acid sequence that is at least 81% identical to SEQ ID NO:19; and (d) a heavy chain variable region polypeptide having an amino acid sequence that is at least 86% identical to SEQ ID NO:21. Sequences are compared for maximum correspondence using a sequence comparison algorithm using the full-length sequence of any one of SEQ ID NO:17, 18, 19, 21, or 23 as the query sequence, as described herein below, or by visual inspection. See also Example 5.

With respect to substantially identical polypeptides having a specified minimal percentage identity to the disclosed humanized H8 variable region polypeptides, substantially identical polypeptides may also be at least about 87% identical to the amino acid sequence of any one of SEQ ID NO:17, 18, 19, 21, or 23, such as at least 88% identical, or at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical. The invention further encompasses polypeptides encoded by any one of the nucleic acids disclosed herein.

Substantially identical proteins also include proteins comprising amino acids that are conservatively substituted variants of any one of the disclosed humanized variable region polypeptides and variable region antibodies. The term conservatively substituted variant refers to a polypeptide comprising an amino acid in which one or more residues have been conservatively substituted with a functionally similar residue and which specifically binds to human anti-5T4 with similar affinity as any of the disclosed chimeric and humanized H8 antibodies. The phrase conservatively substituted variant also includes peptides wherein a residue is replaced with a chemically derivatized residue.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Isolated polypeptides of the invention may be purified and characterized using a variety of standard techniques that are known to the skilled artisan. See e.g., Schröder & Lübke (1965) The Peptides. Academic Press, New York; Bodanszky (1993) Principles of Peptide Synthesis, 2nd rev. ed. Springer-Verlag, Berlin/New York; Ausubel (ed.) (1995) Short Protocols in Molecular Biology, 3rd ed. Wiley, N.Y.

I.C. Nucleotide and Amino Acid Sequence Comparisons

The terms identical or percent identity in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term substantially identical in regards to a nucleotide or polypeptide sequence means that a particular sequence varies from the sequence of a naturally occurring sequence by one or more deletions, substitutions, or additions, the net effect of which is to retain biological function of a humanized anti-5T4 nucleic acid or polypeptide.

For comparison of two or more sequences, typically one sequence acts as a reference sequence to which one or more test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison may be conducted, for example, by the local homology algorithm of Smith & Waterman (1981) Adv. Appl. Math 2:482-489, by the homology alignment algorithm of Needleman & Wunsch (1970) J. Mol. Biol. 48:443-453, by the search for similarity method of Pearson & Lipman (1988) Proc. Natl. Acad. Sci. USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel (ed.) (1995) Short Protocols in Molecular Biology, 3rd ed. Wiley, N.Y.

A preferred algorithm for determining percent sequence identity and sequence similarity is the BLAST algorithm, which is described in Altschul et al. (1990) J. Mol. Biol. 215:403-410. Software for performing BLAST analyses is publicly available through the National Center for Biotechnology Information (http://www.ncbi.nim.nih.gov/). The BLAST algorithm parameters determine the sensitivity and speed of the alignment. For comparison of two nucleotide sequences, the BLASTn default parameters are set at W=11 (wordlength) and E=10 (expectation), and also include use of a low-complexity filter to mask residues of the query sequence having low compositional complexity. For comparison of two amino acid sequences, the BLASTp program default parameters are set at W=3 (wordlength), E=10 (expectation), use of the BLOSUM62 scoring matrix, gap costs of existence=11 and extension=1, and use of a low-complexity filter to mask residues of the query sequence having low compositional complexity. See Example 5.

I.D. Functional Assays

The present invention further discloses in vitro and in vivo assays to characterize activities of a humanized anti-5T4 antibody, including 5T4 binding activity, cellular internalization following binding to 5T4 antigen presented on a cell surface, and targeting to 5T4-expressing cells in a subject. When conjugated to a cytotoxin, the disclosed antibodies of the invention may elicit anti-cancer activity, including inhibition of growth of 5T4-expressing cancer cells and/or induction of cell death in 5T4-expressing cells. Humanized anti-5T4 antibodies of the invention may comprise one or more of the foregoing activities.

Techniques for detecting binding of humanized anti-5T4 antibodies to 5T4 antigen are known in the art, including for example, BIACORE® assays as described in Example 5. Additional representative techniques include centrifugation, affinity chromatography and other immunochemical methods. See e.g., Manson (1992) Immunochemical Protocols, Humana Press, Totowa, N.J., United States of America; Ishikawa (1999) Ultrasensitive and Rapid Enzyme Immunoassay, Elsevier, Amsterdam/New York. Antigen binding assays may be performed using isolated 5T4 antigen or 5T4-expressing cells. See Examples 1 and 5.

The term anti-cancer activity is used to generally describe an ability to destroy existing cancer cells, or to delay or prevent growth of cancer cells. The term cancer refers to both primary and metastasized tumors and carcinomas of any tissue in a subject, including carcinomas and hematopoietic malignancies such as leukemias and lymphomas. In vitro assays for determining anti-cancer activity are described in Examples 2 and 8, and representative animal models are described in Examples 3, 4, and 9.

The term growth inhibitory is used herein to describe an ability of anti-5T4 antibodies to eliminate 5T4-expressing cells or to prevent or reduce proliferation of 5T4-expressing cells. As described in Examples 2-4 and 8-9, humanized anti-5T4 antibodies of the invention may inhibit cancer cell growth. Additional representative methods for rapid in vitro assessment of cell growth inhibition are described in Jones et al. (2001) J. Immunol. Methods 254:85-98.

An ability to induce cell death includes induction of programmed cell death, which is characterized by nuclear DNA degradation, nuclear degeneration and condensation, loss of membrane integrity, and phagocytosis. Representative assays to assess cell are described in Hoves et al. (2003) Methods 31:127-34; Peng et al. (2002) Chin. Med. Sci. J. 17:17-21; Yasuhara et al. (2003) J. Histochem. Cytochem. 51:873-85.

II. Anti-5T4 Antibody/Drug Conjugates

The present invention further provides antibody/drug conjugates comprising a chimeric or humanized anti-5T4 antibody of the invention. Also provided are methods for preparing the antibody/drug conjugates, such that the drug is bound to the antibody either directly or indirectly. Antibody/drug conjugates of the invention have the general formula
5T4Ab(—X—W)ₘ

wherein:

5T4Ab is a chimeric or humanized anti-5T4 antibody or antibody fragment described herein;

X is a linker that comprises a product of any reactive group that may react with an anti-5T4 antibody of antibody fragment;

W is a drug;

m is the average loading for a purified conjugation product (e.g., m such that the drug constitutes about 3 -10% of the conjugate by weight); and

(—X—W)ₘ is a drug derivative.

Also provided are methods for preparing antibody/drug conjugates of the invention. As one example, an antibody/drug conjugate of the formula 5T4Ab(—X—W)ₘ may be prepared by (a) adding the drug derivative to the chimeric or humanized anti-5T4 antibody wherein the drug is 3-10% by weight of the chimeric or humanized anti-5T4 antibody; (b) incubating the drug derivative and the chimeric or humanized anti-5T4 antibody in a non-nucleophilic, protein-compatible, buffered solution having a pH in a range from about 7 to 9 to produce an antibody/drug conjugate, wherein the solution further compromises (i) a suitable organic cosolvent, and (ii) and one or more additives comprising at least one bile acid or its salt, and wherein the incubation is conducted at a temperature ranging from about 30° C. to about 35° C. for a period of time ranging from about 15 minutes to about 24 hours; and (c) subjecting the conjugate produced in step (b) to a chromatographic separation process to separate antibody/drug conjugates with a loading in the range of 3-10% by weight drug and with low conjugated fraction (LCF) from unconjugated chimeric or humanized anti-5T4 antibody, drug derivative, and aggregated conjugates.

II.A. Drugs

The term drug as used herein refers to any substance having biological or detectable activity, for example therapeutic agents, detectable labels, binding agents, etc., and prodrugs, which are metabolized to an active agent in vivo. The term drug also includes drug derivates, wherein a drug has been functionalized to enable conjugation with an antibody of the invention. Generally, these types of conjugates are referred to as immunoconjugates.

The term therapeutic agent refers to any composition that may be used to treat or prevent a condition in a subject in need thereof. In particular, drugs useful in the invention will include anti-cancer drugs. 5T4-expressing cells include cancer cells from squamous/adenomatous lung carcinoma (non-small-cell lung carcinoma), invasive breast carcinoma, colorectal carcinoma, gastric carcinoma, squamous cervical carcinoma, invasive endometrial adenocarcinoma, invasive pancreas carcinoma, ovarian carcinoma, squamous vesical carcinoma, and choriocarcinoma.

Representative therapeutic drugs include cytotoxins, radioisotopes, chemotherapeutic agents, immunomodulatory agents, anti-angiogenic agents, anti-proliferative agents, pro-apoptotic agents, and cytostatic and cytolytic enzymes (e.g., RNAses). A drug may also include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. For example, selected radioisotopes are also cytotoxins. Therapeutic agents may be prepared as pharmaceutically acceptable salts, acids or derivatives of any of the above. Generally, conjugates having a radioisotope as the drug are referred to as radioimmunoconjugates and those having a chemotherapeutic agent as the drug are referred to as chemoimmunoconjugates.

Examples of suitable drugs for use in immunoconjugates include the taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers (i.e., for photodynamic therapy) can also be used in immunoconjugates. In addition, conjugates can be made using secondary carriers as the cytotoxic agent, such as liposomes or polymers, for example.

The term cytotoxin generally refers to an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins. Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluridine, pentostatin, broxuridine, capecitabine, cladribine, decitabine, floxuridine, fludarabine, gougerotin, puromycin, tegafur, tiazofurin, adriamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

In particular embodiments of the invention, the cytotoxin is an antibiotic such as a calicheamicin, also called the LL-E33288 complex, for example, gamma-calicheamicin (γ₁). See U.S. Pat. No. 4,970,198. Early studies with antibody conjugates of gamma calicheamicin hydrazide derivatives showed antigen-based cytotoxicity in vitro and activity in xenograft experiments. The therapeutic index of these conjugates was improved initially by using a less potent derivative, N-acetyl gamma. Stabilizing the disulfide bond that is present in all calicheamicin conjugates by adding dimethyl substituents made additional improvements. Additional examples of calicheamicins suitable for use in preparing antibody/drug conjugates of the invention are disclosed in U.S. Pat. Nos. 4,671,958; 5,053,394; 5,037,651; 5,079,233; and 5,108,912; which are incorporated herein in their entirety. These compounds contain a methyltrisulfide that may be reacted with appropriate thiols to form disulfides, at the same time introducing a functional group such as a hydrazide or other functional group that is useful for conjugating calicheamicin to an anti-5T4 antibody. Stabilizing the disulfide bond that is present in all calicheamicin conjugates by adding dimethyl substituents made additional improvements. This led to the choice of N-acetyl gamma calicheamicin dimethyl hydrazide, or NAc-gamma DMH (CL-184,538), as one of the optimized derivatives for conjugation. Disulfide analogs of calicheamicin can also be used, for example, analogs described in U.S. Pat. Nos. 5,606,040 and 5,770,710, which are incorporated herein in their entirety.

For radiotherapy applications, a chimeric or humanized anti-5T4 antibody of the invention may comprise a high energy radioisotope. The isotope may be directly bound to the antibody, for example, at a cysteine residue present in the antibody, or a chelator may be used to mediate the binding of the antibody and the radioisotope. Radioisotopes suitable for radiotherapy include but are not limited to α-emitters, β-emitters, and auger electrons. For diagnostic applications, useful radioisotopes include positron emitters and γ-emitters. A humanized anti-5T4 antibody of the invention may further be iodinated, for example on a tyrosine residue of the antibody, to facilitate detection or therapeutic effect of the antibody.

Representative radioisotopes that may be conjugated to an anti-5T4 antibody include ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³indium, ^{99m}rhenium, ¹⁰⁵rhenium, ¹⁰¹rhenium, ¹⁸⁶rhenium, ¹⁸⁸rhenium, ¹²¹mtellurium, ⁹⁹technetium, ^{122m}tellurium, ^{125m}tellurium, ¹⁶⁵thulium, ¹⁶⁷thulium, ¹⁶⁸thulium, ⁹⁰yttrium, and nitride or oxide forms derived there from. Other suitable radioisotopes include alpha emitters, such as ²¹³bismuth, ²¹³lead, and ²²⁵actinium.

Angiogenesis and suppressed immune response play a central role in the pathogenesis of malignant disease and tumor growth, invasion, and metastasis. Thus, drugs useful in the methods of the present invention also include those able to induce an immune response and/or an anti-angiogenic response in vivo.

The term immune response is meant to refer to any response to an antigen or antigenic determinant by the immune system of a vertebrate subject, including humoral immune responses (e.g. production of antigen-specific antibodies) and cell-mediated immune responses (e.g. lymphocyte proliferation). Representative immunomodulatory agents include cytokines, xanthines, interleukins, interferons, and growth factors (e.g., TNF, CSF, GM-CSF and G-CSF), and hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Immunomodulatory agents useful in the invention also include anti-hormones that block hormone action on tumors and immunosuppressive agents that suppress cytokine production, downregulate self-antigen expression, or mask MHC antigens. Representative anti-hormones include anti-estrogens including for example tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Representative immunosuppressive agents include 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-IL10 antibodies, anti-TNFα antibodies, anti-IL2 antibodies), streptokinase, TGFβ, rapamycin, T-cell receptor, T-cell receptor fragments, and T cell receptor antibodies.

Representative anti-angiogenic agents include inhibitors of blood vessel formation, for example, farnesyltransferase inhibitors, COX-2 inhibitors, VEGF inhibitors, bFGF inhibitors, steroid sulphatase inhibitors (e.g., 2-methoxyoestradiol bis-sulphamate (2-MeOE2bisMATE)), interleukin-24, thrombospondin, metallospondin proteins, class I interferons, interleukin 12, protamine, angiostatin, laminin, endostatin, and prolactin fragments.

Anti-proliferative agents and pro-apoptotic agents include activators of PPAR-gamma (e.g., cyclopentenone prostaglandins (cyPGs)), retinoids, triterpinoids (e.g., cycloartane, lupane, ursane, oleanane, friedelane, dammarane, cucurbitacin, and limonoid triterpenoids), inhibitors of EGF receptor (e.g., HER4), rampamycin, CALCITRIOL® (1,25-dihydroxycholecalciferol (vitamin D)), aromatase inhibitors (FEMARA® (letrozone)), telomerase inhibitors, iron chelators (e.g., 3-aminopyridine-2-carboxaldehyde thiosemicarbazone (Triapine)), apoptin (viral protein 3—VP3 from chicken aneamia virus), inhibitors of Bcl-2 and Bcl-X(L), TNF-alpha, FAS ligand, TNF-related apoptosis-inducing ligand (TRAIL/Apo2L), activators of TNF-alpha/FAS ligand/TNF-related apoptosis-inducing ligand (TRAIL/Apo2L) signaling, and inhibitors of Pl3K-Akt survival pathway signaling (e.g., UCN-01 and geldanamycin).

Representative chemotherapeutic agents include alkylating agents such as thiotepa and cyclosphosphamide; alkyl sulfonates such as busulfan, improsulfan and piposulfan; aziidines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlornaphazine, cholophosphamide, estramustine, ifosfamide, mechiorethamine, mechiorethamine oxide hydrochloride, melphalan, novembichin, phenesterine, prednimustine, trofosfarnide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-EU; androgens such as calusterone, dromostanolone propionate, epitiostanol, mepitiostane, testolactone; anti-adrenal such as arninoglutethimide, mitotane, trilostane; folic acid replenisher such as frolinic acid; aceglatone; aldophospharnide glycoside; arninolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2′-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g. paclitaxel (TAXOL®, Bristol-Myers Squibb Oncology of Princeton, New Jersey) and doxetaxel (TAXOTERE®, Rhone-Poulenc Rorer of Antony, France); chiorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aininopterin; xeloda; ibandronate; CPT-11; topoisomerase inhibitor RFS 2000; difluoromethylornithine (DMFO); retinoic acid; esperamicins; and capecitabine.

Additional therapeutic agents that may be conjugated to the chimeric and humanized anti-5T4 antibodies disclosed herein and used in accordance with the therapeutic methods of the present invention include but are not limited to photosensitizing agents (U.S. patent Publication No. 2002/0197262 and U.S. Pat. No. 5,952,329) for photodynamic therapy; magnetic particles for thermotherapy (U.S. patent Publication No. 2003/0032995); binding agents, such as peptides, ligands, cell adhesion ligands, etc., and prodrugs such as phosphate-containing prodrugs, thiophosphate-containing prodrugs, sulfate containing prodrugs, peptide containing prodrugs, β-lactam-containing prodrugs, substituted phenoxyacetamide-containing prodrugs or substituted phenylacetamide-containing prodrugs, 5-fluorocytosine and other 5-fluorouridine prodrugs that may be converted to the more active cytotoxic free drug.

For diagnostic methods using chimeric or humanized anti-5T4 antibodies, a drug may comprise a detectable label that may be used to detect the presence of 5T4-expressing cells in vitro or in vivo. Radioisotopes useful for clinical diagnostic applications include labels that are detectable in vivo, such as those labels that are detectable using scintigraphy, magnetic resonance imaging, or ultrasound. Useful scintigraphic labels include positron emitters and γ-emitters. Representative contrast agents for magnetic source imaging are paramagnetic or superparamagnetic ions (e.g., iron, copper, manganese, chromium, erbium, europium, dysprosium, holmium and gadolinium), iron oxide particles, and water soluble contrast agents. For ultrasonic detection, gases or liquids may be entrapped in porous inorganic particles that are released as microbubble contrast agents. For in vitro detection, useful detectable labels include a fluorophore, an epitope, or a radioactive label.

II.B. Linker Molecules

Drugs are conjugated to chimeric and humanized anti-5T4 antibodies of the invention either directly or indirectly via a linker molecule. The linker molecule may be stable or hydrolyzable, whereby it is released following cellular entry. The major mechanisms by which the drug is cleaved from the antibody include hydrolysis in the acidic pH of the lysosomes (hydrazones, acetals, and cis-aconitate-like amides), peptide cleavage by lysosomal enzymes (the cathepsins and other lysosomal enzymes), and reduction of disulfides. As a result of these varying mechanisms for cleavage, mechanisms of linking the drug to the antibody also vary widely and any suitable linker can be used. Preferably, the conjugation method produces a sample with minimal low conjugate fraction (LCF, the fraction of mostly unconjugated antibody), i.e. less than about 10%.

One example of a suitable conjugation procedure relies on the conjugation of hydrazides and other nucleophiles to the aldehydes generated by oxidation of the carbohydrates that naturally occur on antibodies. Hydrazone-containing conjugates can be made with introduced carbonyl groups that provide the desired drug-release properties. Conjugates can also be made with a linker that has a disulfide at one end, an alkyl chain in the middle, and a hydrazine derivative at the other end. The anthracyclines are one example of cytotoxins that can be conjugated to antibodies using this technology.

Linkers containing functional groups other than hydrazones have the potential to be cleaved in the acidic milieu of the lysosomes. For example, conjugates can be made from thiol-reactive linkers that contain a site other than a hydrazone that is cleavable intracellularly, such as esters, amides, and acetals/ketals. Camptothecin is one cytotoxic agent that can be conjugated using these linkers. Ketals made from a 5 to 7-member ring ketone and that has one of the oxygens attached to the cytotoxic agent and the other to a linker for antibody attachment also can be used. The anthracyclines are also an example of a suitable cytotoxin for use with these linkers.

Another example of a class of pH sensitive linkers are the cis-aconitates, which have a carboxylic acid juxtaposed to an amide bond. The carboxylic acid accelerates amide hydrolysis in the acidic lysosomes. Linkers that achieve a similar type of hydrolysis rate acceleration with several other types of structures can also be used. The maytansinoids are an example of a cytotoxin that can be conjugated with linkers attached at C-9.

Another potential release method for drug conjugates is the enzymatic hydrolysis of peptides by the lysosomal enzymes. In on example, a peptide is attached via an amide bond to para-aminobenzyl alcohol and then a carbamate or carbonate is made between the benzyl alcohol and the cytotoxic agent. Cleavage of the peptide leads to the collapse, or self-immolation, of the aminobenzyl carbamate or carbonate. The cytotoxic agents exemplified with this strategy include anthracyclines, taxanes, mitomycin C, and the auristatins. In one example, a phenol can also be released by collapse of the linker instead of the carbamate. In another variation, disulfide reduction is used to initiate the collapse of a para-mercaptobenzyl carbamate or carbonate.

Many of the cytotoxic agents conjugated to antibodies have little, if any, solubility in water and that can limit drug loading on the conjugate due to aggregation of the conjugate. One approach to overcoming this is to add solublizing groups to the linker. Conjugates made with a linker consisting of PEG and a dipeptide can been used, including those having a PEG di-acid, thiol-acid, or maleimide-acid attached to the antibody, a dipeptide spacer, and an amide bond to the amine of an anthracycline or a duocarmycin analogue. Another example is conjugates that are made with a PEG-containing linker disulfide bonded to a cytotoxic agent and amide bonded to an antibody. Approaches that incorporate PEG groups may be beneficial in overcoming aggregation and limits in drug loading.

Representative linkers preferred for preparation of antibody/drug conjugates of the invention include linkers of the formula:
(CO−Alk¹−Sp¹−Ar−Sp²−Alk²−C(Z¹)=Q−Sp)

wherein

Alk¹ and Alk² are independently a bond or branched or unbranched (C₁-C₁₀) alkylene chain;

Sp¹ is a bond, —S—, —O—, —CONH—, —NHCO—, —NR′—, —N(CH₂CH₂)₂N—, or —X—Ar′—Y—(CH₂)ₙ—Z wherein X, Y, and Z are independently a bond, —NR′—, —S—, or —O—, with the proviso that when n=0, then at least one of Y and Z must be a bond and Ar′ is 1,2-, 1,3-, or 1,4-phenylene optionally substituted with one, two, or three groups of (C₁-C₅) alkyl, (C₁-C₄) alkoxy, (C₁-C₄) thioalkoxy, halogen, nitro, —COOR′, —CONHR′, —(CH₂)ₙCOOR′, —S(CH₂)ₙCOOR′, —O(CH₂)ₙCONHR′, or —S(CH₂)ₙCONHR′, with the proviso that when Alk¹ is a bond, Sp¹ is a bond;

n is an integer from 0 to 5;

R′ is a branched or unbranched (C₁-C₅) chain optionally substituted by one or two groups of —OH, (C₁-C₄) alkoxy, (C₁-C₄) thioalkoxy, halogen, nitro, (C₁-C₃) dialkylamino, or (C₁-C₃) trialkylammonium -A⁻ where A⁻ is a pharmaceutically acceptable anion completing a salt;

Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′wherein n and R′ are as hereinbefore defined or a 1,2-, 1,3-, 1,4-, 1,5-, 1,6-, 1,7-, 1,8-, 2,3-, 2,6-, or 2,7-naphthylidene or

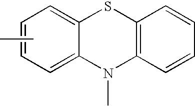

with each naphthylidene or phenothiazine optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, or —S(CH₂)ₙCONHR′ wherein n and R′ are as defined above, with the proviso that when Ar is phenothiazine, Sp¹ is a bond only connected to nitrogen;

Sp² is a bond, —S—, or —O—, with the proviso that when Alk² is a bond, Sp² is a bond;

Z¹ is H, (C₁-C₅) alkyl, or phenyl optionally substituted with one, two, or three groups of (C₁-C₅) alkyl, (C₁-C₅) alkoxy, (C₁-C₄) thioalkoxy, halogen, nitro, —COOR′, —ONHR′, —O(CH₂)ₙCOOR′, —S(CH₂)ₙCOOR′, —O(CH₂)ₙCONHR′, or —S(CH₂)ₙCONHR′ wherein n and R′ are as defined above;

Sp is a straight or branched-chain divalent or trivalent (C₁-C₁₈) radical, divalent or trivalent aryl or heteroaryl radical, divalent or trivalent (C₃-C₁₈) cycloalkyl or heterocycloalkyl radical, divalent or trivalent aryl- or heteroaryl-aryl (C₁-C₁₈) radical, divalent or trivalent cycloalkyl- or heterocycloalkyl-alkyl (C₁-C₁₈) radical or divalent or trivalent (C₂-C₁₈) unsaturated alkyl radical, wherein heteroaryl is preferably furyl, thienyl, N-methylpyrrolyl, pyridinyl, N-methylimidazolyl, oxazolyl, pyrimidinyl, quinolyl, isoquinolyl, N-methylcarbazoyl, aminocourmarinyl, or phenazinyl and wherein if Sp is a trivalent radical, Sp may be additionally substituted by lower (C₁-C₅) dialkylamino, lower (C₁-C₅) alkoxy, hydroxy, or lower (C₁-C₅) alkylthio groups; and

Q is ═NHNCO—, ═NHNCS—, ═NHNCONH—, ═NHNCSNH—, or ═NHO—.

Preferably, Alk¹ is a branched or unbranched (C₁-C₁₀) alkylene chain; Sp′ is a bond, —S—, —O—, —CONH—, —NHCO—, or —NR′ wherein R′ is as hereinbefore defined, with the proviso that when Alk¹ is a bond, Sp¹ is a bond;

Ar is 1,2-, 1,3-, or 1,4-phenylene optionally substituted with one, two, or three groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′ wherein n and R′ are as hereinbefore defined, or Ar is a 1,2-, 1,3-, 1,4-, 1,5-, 1,6-, 1,7-, 1,8-, 2,3-, 2,6-, or 2,7-naphthylidene each optionally substituted with one, two, three, or four groups of (C₁-C₆) alkyl, (C₁-C₅) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′.

Z¹ is (C₁-C₅) alkyl, or phenyl optionally substituted with one, two, or three groups of (C₁-C₅) alkyl, (C₁-C₄) alkoxy, (C₁-C₄) thioalkoxy, halogen, nitro, —COOR′, —CONHR′, —O(CH₂)ₙCOOR′, —S(CH₂)ₙCOOR′, —O(CH₂)ₙCONHR′, or —S(CH₂)ₙCONHR′; Alk² and Sp² are together a bond; and Sp and Q are as immediately defined above.

U.S. Pat. No. 5,773,001, incorporated herein in its entirety, discloses linkers that may be used with nucleophilic drugs, particularly hydrazides and related nucleophiles, prepared from the calicheamicins. These linkers are especially useful in those cases where better activity is obtained when the linkage formed between the drug and the linker is hydrolyzable. These linkers contain two functional groups, including (1) a group for reaction with an antibody (e.g., carboxylic acid), and (2) a carbonyl group (e.g., an aldehyde or a ketone) for reaction with a drug. The carbonyl groups may react with a hydrazide group on the drug to form a hydrazone linkage. This linkage is hydrolyzable, allowing for release of the therapeutic agent from the conjugate after binding to the target cells.

As one example, a chimeric or humanized H8 antibody may be conjugated to a cytotoxic drug by (1) adding the cytotoxic drug derivative to the anti-5T4 antibody wherein the cytotoxic drug is 4.5-11% by weight of the proteinaceous carrier; (2) incubating the cytotoxic drug derivative and anti-5T4 antibody in a non-nucleophilic, protein-compatible, buffered solution having a pH in the range from about 7 to 9 to produce a monomeric cytotoxic drug/antibody conjugate, wherein the solution further comprises (a) a suitable organic cosolvent, and (b) an additive comprising at least one C₆-C₁₈ carboxylic acid or its salt, and wherein the incubation is conducted at a temperature ranging from about 30° C. to about 35° C. for a period of time ranging from about 15 minutes to 24 hours; and (3) subjecting the conjugate produced in step (2) to a chromatographic separation process to separate monomeric conjugates with a loading in the range of 3% to 10% by weight cytotoxic drug and with low conjugated fraction (LCF) below 10 percent from unconjugated antibody, cytotoxic drug derivative, and aggregated conjugates.

The chromatographic separation of step (3) can include processes such as size exclusion chromatography (SEC), ultrafiltration/diafiltration, HPLC, FPLC, or Sephacryl S-200 chromatography. The chromatographic separation may also be accomplished by hydrophobic interaction chromatography (HIC) using Phenyl Sepharose 6 Fast Flow chromatographic medium, Butyl Sepharose 4 Fast Flow chromatographic medium, Octyl Sepharose 4 Fast Flow chromatographic medium, Toyopearl Ether-650M chromatographic medium, Macro-Prep methyl HIC medium or Macro-Prep t-Butyl HIC medium.

Representative methods for preparing anti-H8 antibody/drug conjugates include those described for preparation of CMC-544 in co-pending published U.S. patent application Publication No. 2004-082764A1 and U.S. patent application Ser. No. 10/699,874, which are incorporated herein in their entirety. Conjugation may be performed using the following conditions: 10 mg/ml antibody, 8.5% (w/w) calicheamicin derivative, 37.5 mM sodium decanoate, 9% (v/v) ethanol, 50 mM HEPBS (N-(2-Hydroxyethyl)piperazine-N′-(4-butanesulfonic acid)), pH 8.5, 32° C., 1 hour. Hydrophobic interaction chromatography (HIC) may be performed using a butyl sepharose FF resin, 0.65 M potassium phosphate loading buffer, 0.49 M potassium phosphate wash buffer, and 4 mM potassium phosphate elution buffer. Buffer exchange may be accomplished by size exclusion chromatography, ultrafiltration/diafiltration, or other suitable means. The antibody/drug conjugate may be formulated in 1.5% Dextran-40, 0.9% sucrose, 0.01% TWEEN®-80, 20 mM Tris/50 mM NaCI, pH 8.0. An alternative formulation solution containing 5% sucrose, 0.01 % TWEEN®-80, 20 mM Tris/10 mM NaCl, pH 8.0 may also be used. Lyophilization cycles are adjusted based on the formulation. The concentration of the formulated bulk may be 0.5 mg conjugate/ml. Each may vial contain 1 mg of conjugate, i.e., 2 ml fill. Other fill volumes may be prepared as desired, e.g., 5 ml fill.

Other representative methods include those described for CMD-193, also described in co-pending U.S. patent application Ser. No.11/080,587. Conjugation may be performed using the following conditions: 10 mg/ml antibody, 7% (w/w) calicheamicin derivative, 10 mM deoxycholate, 50 mM HEPBS (N-(2-Hydroxyethyl)piperazine-N′-(4-butanesulfonic acid)), 9% (v/v) ethanol, pH 8.2, 32° C., 1 hour. The reaction may be diluted 10-fold with 0.66 M potassium phosphate pH 8.56, and HIC may be performed using a butyl sepharose FF resin, 0.60 M potassium phosphate loading buffer and wash buffer, and 20 mM Tris/25 mM NaCl elution buffer. Buffer exchange may be accomplished using ultrafiltration/diafiltration with a regenerated cellulose membrane. The conjugate may be diafiltered against 20 mM Tris/10 mM NaCl pH 8.0 (10 diavolumes). The antibody/drug conjugate may be formulated in 5% sucrose, 0.01% TWEEN®80, 20 mM Tris/10 mM NaCl, pH 8.0. The concentration of the bulk conjugate after formulation may be 1 mg/ml, and the vial fill may be 5 mg/vial, i.e., 5 ml fill, or other fill volumes may be prepared as desired.

In a particular embodiment of the invention, the linker employed is 4-(4-acetylphenoxy) butanoic acid (AcBut). Antibody/drug conjugates are prepared by reacting β-calicheamicin, γ-calicheamicin or N-acetyl γ-calicheamicin, or derivatives thereof, with 3-mercapto-3-methyl butanoyl hydrazide, the AcBut linker, and an anti-5T4 antibody of the invention. See e.g., U.S. Pat. No. 5,773,001. This linker produces conjugates that are substantially stable in circulation, releasing an estimated 2% of the NAc-gamma DMH per day, and which release the NAc-gamma DMH readily in the acidic lysosomes. In other embodiments of the invention, antibody/drug conjugates are prepared using 3-acetylphenyl acidic acid (AcPac) or 4-mercapto-4-methyl-pentanoic acid (Amide) as the linker molecule. See Example 2.

Representative linkers useful for conjugation of radioisotopes include diethylenetriamine pentaacetate (DTPA)-isothiocyanate, succinimidyl 6-hydrazinium nicotinate hydrochloride (SHNH), and hexamethylpropylene amine oxime (HMPAO) (Bakker et al. (1990) J. Nucl. Med. 31: 1501-9, Chattopadhyay et al. (2001) Nucl. Med. Biol. 28: 741 -4, Dewanjee et al. (1994) J. Nucl. Med. 35: 1054-63, Krenning et al. (1989) Lancet 1: 242-4, Sagiuchi et al. (2001) Ann. Nucl. Med. 15: 267-70); U.S. Pat. No. 6,024,938). Alternatively, a targeting molecule may be derivatized so that a radioisotope may be bound directly to it (Yoo et al. (1997) J. Nucl. Med. 38: 294-300). Iodination methods are also known in the art, and representative protocols may be found, for example, in Krenning et al. (1989) Lancet 1:242-4 and in Bakker et al. (1990) J. Nucl. Med. 31:1501-9.

To further increase the number of drug molecules per antibody/drug conjugate, the drug may be conjugated to polyethylene glycol (PEG), including straight or branched polyethylene glycol polymers and monomers. A PEG monomer is of the formula: —(CH₂CH₂O)—. Drugs and/or peptide analogs may be bound to PEG directly or indirectly, i.e. through appropriate spacer groups such as sugars. A PEG/antibody/drug composition may also include additional lipophilic and/or hydrophilic moieties to facilitate drug stability and delivery to a target site in vivo. Representative methods for preparing PEG-containing compositions may be found in U.S. Pat. Nos. 6,461,603; 6,309,633; and 5,648,095, among other places.

The hydrophobic nature of many drugs, including calicheamicins, may results in aggregation of antibody/drug conjugates. To produce monomeric antibody/drug conjugates with higher drug loading/yield and decreased aggregation, the conjugation reaction may be performed in a non-nucleophilic, protein-compatible, buffered solution containing (i) propylene glycol as a cosolvent and (ii) an additive comprising at least one C₆-C₁₈ carboxylic acid. Useful acids include C₇ to C₁₂ acids, such as octanoic acid or caprylic acid, or its salts. Other protein-compatible organic cosolvents other than propylene glycol, such as ethylene glycol, ethanol, DMF, DMSO, etc., may also be used. Some or all of the organic cosolvent is used to transfer the drug into the conjugation mixture. Useful buffers for the preparation of antibody/drug conjugates using N-hydroxysuccinimide (OSu) esters or other comparably activated esters include phosphate-buffered saline (PBS) and N-2-hydroxyethyl piperazine-N′-2-ethanesulfonic acid (HEPES buffer). The buffered solution used in conjugation reactions should substantially lack free amines and nucleophiles. As another approach, the conjugation reactions may be performed in a non-nucleophilic, protein-compatible, buffered solution containing t-butanol without the additional additives. See e.g., U.S. Pat. Nos. 5,712,374 and 5,714,586. Additional methods for conjugation and calicheamicin-containing conjugates are described in U.S. Pat. Nos. 5,739,116 and 5,877,296.

Optimal reaction conditions for formation of a monomeric conjugate may be empirically determined by variation of reaction variables such as temperature, pH, calicheamicin derivative input, and additive concentration. Representative amounts of propylene glycol range from 10% to 60%, for example 10% to 40%, or about 30% by volume of the total solution. Representative amounts of an additive comprising at least one C₆-C₁₈ carboxylic acid or its salt range from 20 mM to 100 mM, such as from 40 mM to 90 mM, or about 60 mM to 90 mM. The concentration of the C₆-C₁₈ carboxylic acid or its salt may be increased to 150-300 mM and the cosolvent dropped to 1% to 10%. In representative embodiments of the invention, the carboxylic acid is octanoic acid, decanoic acid, or the corresponding salts. For example, 200 mM caprylic acid may be used with 5% propylene glycol or ethanol. The conjugation reaction may be performed at slightly elevated temperature (30-35° C.) and pH (8.2-8.7). The concentration of antibody may range from 1 to 15 mg/ml and the concentration of a calicheamicin derivative, e.g., N-Acetyl gamma-calicheamicin DMH AcBut OSu ester may range from about 4.5% to 11% by weight of the antibody. Conditions suitable for conjugation of other drugs may be determined by those skilled in the art without undue experimentation.

II.C. Purification of Antibody/Drug Conjugates

Following conjugation, the monomeric conjugates may be separated from unconjugated reactants and/or aggregated forms of the conjugates by conventional methods, for example size exclusion chromatography (SEC), hydrophobic interaction chromatography (HIC), ion exchange chromatography (IEC), or chromatofocusing (CF). The purified conjugates are monomeric, and usually contain from 3% to 10% drug by weight. Antibody/drug conjugates may also be purified using hydrophobic interaction chromatography (HIC), which offers some advantages over SEC including (1) a capability to efficiently reduce the LCF content as well as aggregate; (2) accommodation of large reaction volumes; and (3) minimal dilution of the product. High-capacity HIC media suitable for production scale use include Phenyl Sepharose 6 Fast Flow chromatographic medium, Butyl Sepharose 4 Fast Flow chromatographic medium, Octyl Sepharose 4 Fast Flow chromatographic medium, Toyopearl Ether-650M chromatographic medium, Macro-Prep methyl HIC medium or Macro-Prep t-Butyl HIC medium. Ultrafiltration/diafiltration may also be used for buffer exchange.

In a representative purification process, multiple steps are performed, including a centrifuge cell removal step, a Protein A affinity capture step followed by one or two orthogonal chromatographic polishing steps, a virus filtration step, and a tangential-flow filtration step for concentration and formulation. The purification process preferably yields product with less than 5% aggregate, less than 20 ppm Protein A, less than 50 ppm host cell protein, and overall recovery of greater than 50%.

A typical humanized anti-5T4/calicheamicin preparation contains predominantly (˜95%) conjugated antibody containing 5-7 moles calicheamicin per mole antibody. The conjugate has been reproducibly prepared at the laboratory scale (10-200 mg). Drug loading, which is expressed as μg calicheamicin/mg mAb, is determined by dividing the calicheamicin concentration (μg/mL) by the antibody concentration (mg/mL). These values are determined by measuring the UV absorbance of the conjugate solution at 280 nm and 310 nm. It is important to note that this is an average loading and that the actual loading is a quasi-gaussian distribution centered on the average loading value, i.e., some of the antibody is loaded higher than average and some of the antibody is loaded lower than the average. Unconjugated antibody (low conjugated fraction), which can be measured using analytical HIC-HPLC (hydrophobic interaction high-performance liquid chromatography), is the population of antibody that has little or no conjugated calicheamicin. This value is a measure of calicheamicin distribution on the antibody and does not generally affect the amount of calicheamicin dosed. Unconjugated calicheamicin, which can be measured using ELISA, refers to the amount of calicheamicin that is not conjugated to the antibody and is expressed in terms of percent of total calicheamicin. Drug-loading assays do not differentiate between unconjugated and conjugated calicheamicin. The amount of unconjugated calicheamicin is undetectable or negligible when using drug-loading assays, and therefore these assays effectively measure the amount of conjugated calicheamicin.

Analytical methods can be used to assay for release and stability testing of humanized anti-5T4 calicheamicin conjugates. The conjugates can be evaluated for identity (IEF), strength (total protein and total calicheamicin loading), purity (unconjugated calicheamicin, low conjugated antibody, aggregate content and SDS-PAGE Reduced), and immunoaffinity (antigen binding ELISA). Additional assays known to those of skill in the art can be used. Using these assays, batch-to-batch consistency can be maintained in commercial manufacture.

II.D. Pharmacokinetics of Antibody/Drug Conjugates

The pharmacokinetics of 5T4-targeted immunoconjugates can be evaluated and compared to the pharmacokinetics of unconjugated calicheamicin in various animals. For example, this can be done following a single intravenous bolus administration in female nude mice, male Sprague-Dawley rats, and female cynomolgus monkeys. Pharmacokinetics of an anti-5T4 antibody are generally characterized by low clearance, low volume of distribution, and long apparent terminal half-life in various species. The serum concentrations of unconjugated calicheamicin derivatives are expected to be below the quantification limit. The toxicity profile for these conjugates in single-dose toxicity ranging studies is expected to be similar to that obtained for other antibody/calicheamicin conjugates at comparable doses.

III. Uses of Chimeric and Humanized Anti-5T4 Antibodies and Antibody/Drug Conjugates

The humanized anti-5T4 antibodies and antibody/drug conjugates of the invention are useful both in vitro and in vivo for applications related to 5T4-expressing cells. As described in Example 1, 5T4expressing cancer cells include squamous/adenomatous lung carcinoma (non-small-cell lung carcinoma), invasive breast carcinoma, colorectal carcinoma, gastric carcinoma, squamous cervical carcinoma, invasive endometrial adenocarcinoma, invasive pancreas carcinoma, ovarian carcinoma, squamous vesical carcinoma, and choriocarcinoma. 5T4 was detected at high levels on carcinomas of bronchi, breast, colon, rectum, stomach, cervix, endometrium, pancreas, ovaria, chorium, and seminal vesicles.

For in vivo applications, the utility of the disclosed humanized antibodies as drug carriers relies on their ability to behave as targeting molecules. The term targeting refers to the preferential movement and/or accumulation of a peptide or peptide analog in a target tissue as compared with a control tissue. The term target tissue as used herein refers to tissues comprising 5T4-expressing cells, i.e., an intended site for accumulation of an antibody/drug conjugate of the invention following administration to a subject. The term control tissue as used herein refers to a site suspected to substantially lack binding and/or accumulation of an administered antibody/drug conjugate, i.e., tissues that substantially lack 5T4-expressing cells. The term selective targeting is used herein to refer to a preferential localization of an antibody/drug conjugate such that an amount of antibody/drug conjugate in a target tissue is about 2-fold greater than an amount of peptide analog in a control tissue, such as an amount that is about 5-fold or greater, or about 10-fold or greater.

III.A. In Vitro Applications

The present invention provides in vitro methods using humanized anti-5T4 antibodies. For example, the disclosed antibodies may be used, either alone or in combination with cytotoxic agents or other drugs to specifically bind 5T4-positive cancer cells to deplete such cells from a cell sample.

Methods are also provided for targeted cytolysis via contacting 5T4-expressing cells with an antibody/drug conjugate comprising an anti-5T4 antibody conjugated to a cytotoxin. See Examples 3 and 8. Also provided are methods for inhibiting proliferation of 5T4-expressing cells and for inducing apoptosis of 5T4-expressing cells via contacting the cells with an antibody/drug conjugate comprising a cytotoxic drug. The contacting of the 5T4-expressing cells with the antibody/drug conjugate may be accomplished in vitro or in vivo.

III.B. Diagnostic and Detection Methods

Humanized anti-5T4 antibodies of the invention also have utility in the detection of 5T4+ cells in vitro and in vivo based on their ability to specifically bind the 5T4 antigen. A method for detecting 5T4-expressing cells may comprise: (a) preparing a biological sample comprising cells; (b) contacting a humanized anti-5T4 antibody with the biological sample in vitro, wherein the antibody comprises a detectable label; and (c) detecting the detectable label, whereby 5T4-expressing cells are detected.

The disclosed detection methods may also be performed in vivo, for example as useful for diagnosis, to provide intraoperative assistance, or for dose determination. Following administration of a labeled humanized anti-5T4 antibody to a subject, and after a time sufficient for binding, the biodistribution of 5T4-expressing cells bound by the antibody may be visualized. The disclosed diagnostic methods may be used in combination with treatment methods. In addition, humanized anti-5T4 antibodies of the invention may be administered for the dual purpose of detection and therapy.

Representative non-invasive detection methods include scintigraphy (e.g., SPECT (Single Photon Emission Computed Tomography), PET (Positron Emission Tomography), gamma camera imaging, and rectilinear scanning), magnetic resonance imaging (e.g., convention magnetic resonance imaging, magnetization transfer imaging (MTI), proton magnetic resonance spectroscopy (MRS), diffusion-weighted imaging (DWI) and functional MR imaging (fMRI)), and ultrasound.

III.C. Therapeutic Applications

The present invention further relates to methods and compositions useful for inducing cytolysis of 5T4-expressing cancer cells in a subject. Thus, the disclosed methods are useful for inhibiting cancer growth, including delayed tumor growth and inhibition of metastasis. While not intending to be bound by any single mode of operation, both antigen-guided targeting (see e.g., Examples 3, 4, and 9) as well as passive targeting (see e.g., Example 10) of humanized H8-calicheamicin conjugates may contribute to anti-tumor efficacy.

Representative cancers treatable using the disclosed anti-5T4 antibodies and antibody/drug conjugates include 5T4-expressing primary and metastatic tumors in breast, colon, rectum, lung, oropharynx, hypopharynx, esophagus, stomach, pancreas, liver, gallbladder, bile ducts, small intestine, urinary tract including kidney, bladder and urothelium, female genital tract, cervix, uterus, ovaries, male genital tract, prostate, seminal vesicles, testes, an endocrine gland, thyroid gland, adrenal gland, pituitary gland, skin, bone, soft tissues, blood vessels, brain, nerves, eyes, meninges. In particular, the disclosed anti-5T4 antibody/drug conjugates of the invention may be used for the treatment of non-small cell lung cancer, metastatic breast cancer, and pancreatic cancer as both second-line monotherapy and as part of first-line combination therapy. Target cancers may also express the Lewis Y carbohydrate antigen, including breast, colon, gastric, esophageal, pancreatic, duodenal, lung, bladder and renal carcinomas and gastric and islet cell neuroendocrine tumors. See U.S. Pat. No. 6,310,185.

The disclosed methods also pertain to 5T4-expressing leukemia and lymphoma cells, including Hodgkin's lymphoma cells and non-Hodgkin's lymphoma cells. The lymphoma cells may be lymphoma cells indolent, aggressive, low-grade, intermediate-grade, or high-grade.

Thus, patients to be treated with the humanized anti-5T4 antibody/calicheamicin conjugates of the invention may be selected based on biomarker expression, including but not limited to elevated expression of 5T4 antigen, resulting in a patient population selected for enriched target expression rather than tumor origin or histology. Target expression can be measured as a function of the number of cells staining combined with the intensity of the cells staining. For example, classification of high expression of 5T4 includes those patients with greater than 30% (i.e., 40%, 50% or 60%) of the cells tested by immunohistochemical staining positive for 5T4 at a level of 3+ (on a scale of 1 to 4), while moderate expression of the 5T4 can include those patients with greater than 20% of the cell cells staining at 1+ to 2+.

Biomarkers other than expression of 5T4 antigen can be also used for patient selection, including characterization of the tumor based on multi-drug resistence (MDR), for example. Nearly 50 per cent of human cancers are either completely resistant to chemotherapy or respond only transiently, after which they are no longer affected by commonly used anticancer drugs. This phenomenon is referred to as MDR and is inherently expressed by some tumor types, while others acquire MDR after exposure to chemotherapy treatment. The drug efflux pump P-glycoprotein mediates a majority of the MDR associated with cytotoxic chemotherapeutics. Phenotypic and functional analysis of MDR mechanisms present in cancer patient tumor specimens can be conducted in order to relate specific MDR mechanism(s) with resistance to chemotherapy in specific tumor types.

The term cancer, as used herein, also encompasses non-neoplastic proliferative disorders. Thus, the methods of the present invention are contemplated for the treatment or prevention of hyperplasia, metaplasia, or most particularly, dysplasia (for review of such abnormal growth conditions, see DeVita, Jr. et a. (2001), Cancer: Principles and Practice, 6^{th} edition, Lippincott Williams & Wilkins.

The term cancer growth generally refers to any one of a number of indices that suggest change within the cancer to a more developed form. Thus, indices for measuring an inhibition of cancer growth include but are not limited to a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. The term delayed tumor growth refers to a decrease in duration of time required for a tumor to grow a specified amount. For example, treatment may delay the time required for a tumor to increase in volume 3-fold relative to an initial day of measurement (day 0) or the time required to grow to 1 cm³.

III.D. Formulation

Chimeric and humanized anti-5T4 antibodies of the invention are readily prepared and formulated for safe and efficacious clinical use. Suitable formulations for administration to a subject include aqueous and non-aqueous sterile injection solutions which may contain anti-oxidants, buffers, bacteriostats, antibacterial and antifungal agents (e.g., parabens, chlorobutanol, phenol, ascorbic acid, and thimerosal), solutes that render the formulation isotonic with the bodily fluids of the intended recipient (e.g., sugars, salts, and polyalcohols), suspending agents and thickening agents. Suitable solvents include water, ethanol, polyol (e.g., glycerol, propylene glycol, and liquid polyethylene glycol), and mixtures thereof. The formulations may be presented in unit-dose or multi-dose containers, for example sealed ampoules and vials, and may be stored in a frozen or freeze-dried (lyophilized) condition requiring only the addition of sterile liquid carrier immediately prior to use for administration to a subject or for subsequent radiolabeling with an isotope appropriate for the intended application. Anti-5T4 antibodies and antibody/drug conjugates of the invention are preferably formulated as an effective dose, described below.

As one example, a representative anti-5T4 antibody formulation comprises a multi-dose formulation of 40 mg/ml antibody or antibody/drug conjugate, 25 mM acetate, 150 mM trehalose, 0.9% benzyl alcohol, 0.02% polysorbate 20 at pH 5.0, and which has a minimum shelf life of two years storage at 2-8° C. As another example, an anti-5T4 antibody formulation may comprise 10 mg/ml antibody or antibody/drug conjugate in 9.0 mg/ml sodium chloride, 7.35 mg/ml sodium citrate dihydrate, 0.7 mg/ml polysorbate 80, and sterile water, pH 6.5. Representative formulations of an anti-5T4/calicheamicin conjugate for administration to experimental mouse models include 2 μg or 4 μg calicheamicin (see Examples 3, 4, and 7), which may be scaled accordingly for administration to humans.

A stable lyophilized formulation of an anti-5T4 antibody or antibody/drug conjugate may be prepared by (a) dissolving an antibody/drug conjugate to a final concentration of 0.5 to 2 mg/ml in a solution comprising a cryoprotectant at a concentration of 1.5%-5% by weight, a polymeric bulking agent at a concentration of 0.5-1.5% by weight, electrolytes at a concentration 0.01 M to 0.1 M, a solubility facilitating agent at a concentration of 0.005% to 0.05% by weight, buffering agent at a concentration of 5-50 mM such that the final pH of the solution is 7.8-8.2, and water; (b) dispensing the above solution into vials at a temperature of +5° C. to +10° C.; (c) freezing the solution at a freezing temperature of −35° C. to −50° C.; (d) subjecting the frozen solution to an initial freeze drying step at a primary drying pressure of 20 to 80 microns at a shelf temperature at −10° C. to −40° C. for 24 to 78 hours; and (e) subjecting the freeze-dried product of step (d) to a secondary drying step at a drying pressure of 20 to 80 microns at a shelf temperature of +10° C. to +35° C. for 15 to 30 hours.

Representative cryoprotectants useful for lyophilization of the cryoprotectant include alditol, mannitol, sorbitol, inositol, polyethylene glycol, aldonic acid, uronic acid, aldaric acid, aldoses, ketoses, amino sugars, alditols, inositols, glyceraldehydes, arabinose, lyxose, pentose, ribose, xylose, galactose, glucose, hexose, idose, mannose, talose, heptose, glucose, fructose, gluconic acid, sorbitol, lactose, mannitol, methyl a-glucopyranoside, maltose, isoascorbic acid, ascorbic acid, lactone, sorbose, glucaric acid, erythrose, threose, arabinose, allose, altrose, gulose, idose, talose, erythrulose, ribulose, xylulose, psicose, tagatose, glucuronic acid, gluconic acid, glucaric acid, galacturonic acid, mannuronic acid, glucosamine, galactosamine, sucrose, trehalose, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthan gum, starch, sucrose, glucose, lactose, trehalose, ethylene glycol, polyethylene glycol, polypropylene glycol, glycerol and pentaerythritol.

For example, the cryoprotectant sucrose may be used at a concentration of 1.5% by weight, the polymeric bulking agent Dextran 40 or hydroxyethyl starch 40 may be used at a concentration of 0.9% by weight, the electrolyte used in the lyophilization solution is sodium chloride, which is present at a concentration of 0.05 M, and the buffering agent tromethamine may be used at a concentration of 0.02 M. A solubility facilitating agent (e.g., a surfactant such as Polysorbate 80) may also be used during the lyophilization process. Usually this solubility facilitating agent is a surfactant. Representative steps for preparation of a lyophilized formulation include freezing the vials at a temperature of −45° C.; the frozen solution is subjected to an initial freeze drying step at a primary drying pressure of 60 microns and at a shelf temperature of −30° C. for 60 hours; and subjecting the freeze-dried product to a secondary drying step at a drying pressure of 60 microns at a shelf temperature of +25° C. for 24 hours.

Anti-5T4 antibodies and antibody/drug conjugates are formulated in a pharmaceutically acceptable carrier, for example, large slowly metabolized macromolecules such as proteins, polypeptides, liposomes, polysaccharides, polylactic acids, polyglycolic acids, polymeric amino acids, amino acid copolymers and inactive virus particles. Pharmaceutically acceptable salts may also be used, for example, mineral acid salts, such as hydrochlorides, hydrobromides, phosphates and sulfates, or salts of organic acids, such as acetates, propionates, malonates and benzoates. Formulations may additionally contain liquids such as water, saline, glycerol, and ethanol, and/or auxiliary substances, such as wetting or emulsifying agents or pH buffering substances, may be present in such compositions. Such carriers enable the compositions to be formulated as tablets, pills, dragees, capsules, liquids, gels, syrups, slurries and suspensions, for ingestion by the patient.

III.E. Dose and Administration

A humanized anti-5T4 antibody may be administered parenterally, for example, via intravascular, subcutaneous, intraperitoneal, or intramuscular administration. For delivery of compositions to pulmonary pathways, compositions may be administered as an aerosol or coarse spray, i.e. transnasal administration. Intrathecal or intramedullary administration may be used for treatment of central nervous system (CNS) and CNS-related cancers. An anti-5T4 antibody of the invention may also be administered transdermally, transcutaneously, topically, enterally, intravaginally, sublingually or rectally. A delivery method is selected based on considerations such as the condition and site to be treated, the type of antibody formulation, and the therapeutic efficacy of the composition. Intravenous administration may be routinely used in the clinic.

The present invention provides that an effective amount of a humanized anti-5T4 antibody is administered to a subject. The term effective amount is used herein to describe an amount of a humanized anti-5T4 antibody sufficient to elicit a desired biological response. For example, when administered to a cancer-bearing subject, an effective amount comprises an amount sufficient to elicit an anti-cancer activity, including cancer cell cytolysis, inhibition of cancer cell proliferation, induction of cancer cell apoptosis, reduction of cancer cell antigens, delayed tumor growth, and inhibition of metastasis. Tumor shrinkage is well accepted as a clinical surrogate marker for efficacy. Another well accepted marker for efficacy is progression-free survival. Anti-5T4/calicheamicin conjugates generally demonstrate at least a 25% improvement in key efficacy parameters, such as improvement in median survival, time to tumor progression, and overall response rate.

Generally, an effective dose is in the range from about 0.01 mg/m² to about 50 mg/m², such as from about 0.1 mg/m² to about 20 mg/m², or about 15 mg/m², which dose is calculated based on the amount of anti-5T4 antibody or based upon the amount of calicheamicin in the antibody/calicheamicin preparation. Representative doses of an anti-5T4/calicheamicin conjugate for administration to experimental mouse models include 2 μg or 4 μg calicheamicin (see Examples 3-4 and 9), which may be scaled accordingly for administration to humans. For example, anti-5T4/calicheamicin conjugates of the invention may be administered to human patients once every 3 weeks for up to 6 cycles. For a radiolabeled anti-5T4 antibody, an effective dose is typically in the range from about 1 mCi to about 300 mCi, normally about 5 mCi to 100 mCi, depending on the radioisotope and the binding affinity of the antibody.

For detection of 5T4-positive cells using the disclosed chimeric and humanized anti-5T4 antibodies, a detectable amount of a composition of the invention is administered to a subject. A detectable amount, as used herein to refer to a diagnostic composition, refers to a dose of a chimeric or humanized H8 antibody such that the presence of the antibody may be determined in vitro or in vivo. For scintigraphic imaging using radioisotopes, typical doses of a radioisotope may include an activity of about 10 μCi to 50 mCi, or about 100 μCi to 25 mCi, or about 500 μCi to 20 mCi, or about 1 mCi to 10 mCi, or about 10 mCi.

Actual dosage levels of active ingredients in a composition of the invention may be varied so as to administer an amount of the composition that is effective to achieve the desired diagnostic or therapeutic outcome. Administration regimens may also be varied. A single injection or multiple injections may be used. The selected dosage level and regimen will depend upon a variety of factors including the activity and stability (i.e., half life) of the therapeutic composition, formulation, the route of administration, combination with other drugs or treatments, the disease or disorder to be detected and/or treated, and the physical condition and prior medical history of the subject being treated.

For any anti-5T4 or antibody/drug conjugate of the invention, the therapeutically effective dose may be estimated initially either in cell culture assays or in animal models, usually in rodents, rabbits, dogs, pigs, and/or or primates. The animal model may also be used to determine the appropriate concentration range and route of administration. Such information may then be used to determine useful doses and routes for administration in humans. Typically a minimal dose is administered, and the dose is escalated in the absence of dose-limiting cytotoxicity. Determination and adjustment of an effective amount or dose, as well as evaluation of when and how to make such adjustments, are known to those of ordinary skill in the art of medicine.

For additional guidance regarding formulation, dose, administration regimen, and measurable therapeutic outcomes, see Berkow et al. (2000) The Merck Manual of Medical Information, Merck & Co., Inc., Whitehouse Station, N.J.; Ebadi (1998) CRC Desk Reference of Clinical Pharmacology, CRC Press, Boca Raton, Fla.; Gennaro (2000) Remington: The Science and Practice of Pharmacy, Lippincott, Williams & Wilkins, Philadelphia, Pa.; Katzung (2001) Basic & Clinical Pharmacology, Lange Medical Books/McGraw-Hill Medical Pub. Div., New York; Hardman et al. (2001) Goodman & Gilman's the Pharmacological Basis of Therapeutics, The McGraw-Hill Companies, Columbus, Ohio; Speight & Holford (1997) Avery's Drug Treatment: A Guide to the Properties, Choices, Therapeutic Use and Economic Value of Drugs in Disease Management, Lippincott, Williams, & Wilkins, Philadelphia, Pa.

III.F. Combination Therapies

The disclosed anti-5T4 antibodies may be administered as an initial treatment, or for treatment of conditions that are unresponsive to conventional therapies. In addition, the disclosed anti-5T4 antibodies may be used in combination with other therapies (e.g., surgical excision, radiation, additional targeted anti-cancer drugs or systemic anti-cancer drugs, etc.) to thereby elicit additive or potentiated therapeutic effects and/or reduce hepatocytotoxicity of some anti-cancer agents. Chimeric and humanized anti-5T4 antibodies of the invention may be co-administered or co-formulated with additional agents, or formulated for consecutive administration in either order.

Representative agents useful for combination therapy include any of the drugs described herein above as useful for preparation of anti-5T4/drug conjugates. Chimeric and humanized anti-5T4 antibodies of the invention may also be used in combination with other therapeutic antibodies and antibody/drug conjugates, including anti-5T4 antibodies other than the disclosed humanized anti-5T4 antibodies, as well as anti-CD19, anti-CD20 (e.g., RITUXAN®, ZEVALIN®, BEXXAR®), anti-CD22 antibodies, anti-CD33 antibodies (e.g., MYLOTARG®), anti-CD33 antibody/drug conjugates, anti-Lewis Y antibodies (e.g., Hu3S193, Mthu3S193, AGmthu3S193), anti-HER-2 antibodies (e.g., HERCEPTIN® (trastuzumab), MDX-210, OMNITARG® (pertuzumab, rhuMAb 2C4)), anti-CD52 antibodies (e.g., CAMPATH®), anti-EGFR antibodies (e.g., ERBITUX® (cetuximab), ABX-EGF (panitumumab)), anti-VEGF antibodies (e.g., AVASTIN® (bevacizumab)), anti-DNA/histone complex antibodies (e.g., ch-TNT-1/b), anti-CEA antibodies (e.g., CEA-Cide, YMB-1003) hLM609, anti-CD47 antibodies (e.g., 6H9), anti-VEGFR2 (or kinase insert domain-containing receptor, KDR) antibodies (e.g., IMC-1C11), anti-Ep-CAM antibodies (e.g., ING-1), anti-FAP antibodies (e.g., sibrotuzumab), anti-DR4 antibodies (e.g., TRAIL-R), anti-progesterone receptor antibodies (e.g., 2C5), anti-CA19.9 antibodies (e.g., GIVAREX®) and anti-fibrin antibodies (e.g., MH-1).

Anti-5T4 antibody/drug conjugates may also be administered together with one or more combinations of cytotoxic agents as part of a treatment regimen. Useful cytotoxic preparations for this purpose include CHOPP (cyclophosphamide, doxorubicin, vincristine, prednisone and procarbazine); CHOP (cyclophosphamide, doxorubicin, vincristine, and prednisone); COP (cyclophosphamide, vincristine, prednisone); CAP-BOP (cyclophosphamide, doxorubicin, procarbazine, bleomycin, vincristine and prednisone); m-BACOD (methotrexate, bleomycin, doxorubicin, cyclophosphamide, vincristine, dexamethasone, and leucovorin; ProMACE-MOPP (prednisone, methotrexate, doxorubicin, cyclophosphamide, etoposide, leukovorin, mechloethamine, vincristine, prednisone and procarbazine); ProMACE-CytaBOM (prednisone, methotrexate, doxorubicin, cyclophosphamide, etoposide, leukovorin, cytarabine, bleomycin and vincristine); MACOP-B (methotrexate, doxorubicin, cyclophosphamide, vincristine, prednisone, bleomycin and leukovorin); MOPP (mechloethamine, vincristine, prednisone and procarbazine); ABVD (adriamycin/doxorubicin, bleomycin, vinblastine and dacarbazine); MOPP (mechloethamine, vincristine, prednisone and procarbazine) alternating with ABV (adriamycin/doxorubicin, bleomycin, vinblastine); MOPP (mechloethamine, vincristine, prednisone and procarbazin) alternating with ABVD(adriamycin/doxorubicin, bleomycin, vinblastine and dacarbazine); ChIVPP (chlorambucil, vinblastine, procarbazine, prednisone); IMVP-16 (ifosfamide, methotrexate, etoposide); MIME (methyl-gag, ifosfamide, methotrexate, etoposide); DHAP (dexamethasone, high-dose cytaribine and cisplatin); ESHAP (etoposide, methylpredisolone, HD cytarabine, and cisplatin); CEPP(B) (cyclophosphamide, etoposide, procarbazine, prednisone and bleomycin); CAMP (lomustine, mitoxantrone, cytarabine and prednisone); and CVP-1 (cyclophosphamide, vincristine and prednisone); DHAP (cisplatin, high-dose cytarabine and dexamethasone); CAP (cyclophosphamide, doxorubicin, cisplatin); PV (cisplatin, vinblastine or vindesine); CE (carboplatin, etoposide); EP (etoposide, cisplatin); MVP (mitomycin, vinblastine or vindesine, cisplatin); PFL (cisplatin, 5-flurouracil, leucovorin); IM (ifosfamide, mitomycin); IE (ifosfamide, etoposide); IP (ifosfamide, cisplatin); MIP (mitomycin, ifosfamide, cisplatin); ICE (ifosfamide, carboplatin, etoposide); PIE (cisplatin, ifosfamide, etoposide); Viorelbine and cisplatin; Carboplatin and paclitaxel; CAV (cyclophosphamide, doxorubicin, vincristine); CAE (cyclophosphamide, doxorubicin, etoposide); CAVE (cyclophosphamide, doxorubicin, vincristine, etoposide); EP (etoposide, cisplatin); and CMCcV (cyclophosphamide, methotrexate, lomustine, vincristine).

Anti-5T4/calicheamicin conjugates may be used in combination with systemic anti-cancer drugs, such as epithilones (BMS-247550, Epo-906), reformulations of taxanes (Abraxane, Xyotax), microtubulin inhibitors (MST-997, TTI-237), or with targeted cytotoxins such as CMD-193 and SGN-15. Additional useful anti-cancer agents include TAXOTERE®, TARCEVA®, GEMZAR® (gemcitabine), 5-FU, AVASTIN®, ERBITUX®, TROVAX®, anatumomab mafenatox, letrazole, docetaxel, and anthracyclines.

For combination therapies, a humanized anti-5T4 antibody and additional therapeutic or diagnostic agents are administered within any time frame suitable for performance of the intended therapy or diagnosis. Thus, the single agents may be administered substantially simultaneously (i.e., as a single formulation or within minutes or hours) or consecutively in any order. For example, single agent treatments may be administered within about 1 year of each other, such as within about 10, 8, 6, 4, or 2 months, or within 4, 3, 2 or 1 week(s), or within about 5, 4, 3, 2 or 1 day(s). The administration of the anti-5T4/calicheamicin conjugate and the second therapeutic agent preferably elicits a greater effect than administration of either alone.

### EXAMPLES

The following examples have been included to illustrate modes of the invention. Certain aspects of the following examples are described in terms of techniques and procedures found or contemplated by the present co-inventors to work well in the practice of the invention. These examples illustrate standard laboratory practices of the co-inventors. In light of the present disclosure and the general level of skill in the art, those of skill will appreciate that the following examples are intended to be exemplary only and that numerous changes, modifications, and alterations may be employed without departing from the scope of the invention.

### Example 1

### 5T4 Expression in Normal and Malignant Tissues

To consider 5T4 antigen as a target for cancer therapy, the distribution of 5T4 on normal and malignant tissues was determined. 5T4 was observed at high levels on the surface of various tumor cells, in some cases correlating with progression of the disease, and was substantially absent from most normal cells. This expression profile suggested 5T4 as a plausible target for cancer immunotherapy.

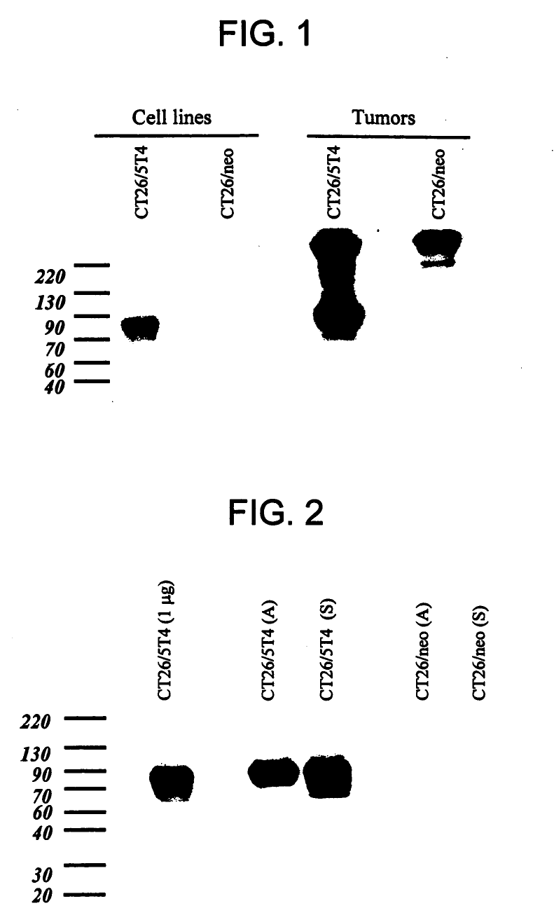

The expression of 5T4 in normal and cancerous tissues was assayed using the murine H8 anti-5T4 antibody according to standard techniques such as Western blot. Affinity of various antibodies and conjugates for 5T4 was verified by plasmon resonance or FACS analysis. H8 is a hybridoma generated monoclonal mouse IgG1 antibody which is described in PCT International Publication No. WO 98/55607 and in Forsberg et al. (1997) J. Biol. Chem. 272(19):124430-12436. For use as a positive control in in vitro and in vivo assays, tumor cells that express reproducibly high levels of 5T4 were established by transfecting a vector expressing 5T4 into CT26 mouse colon carcinoma and MDAMB435 human breast carcinoma cells. See FIG. 1 and Table 1.

Tumor samples tested included squamous/adenomatous lung carcinoma (non-small-cell lung carcinoma), invasive breast carcinoma, colorectal carcinoma, gastric carcinoma, squamous cervical carcinoma, invasive endometrial adenocarcinoma, invasive pancreas carcinoma, ovarian carcinoma, squamous vesical carcinoma, and choriocarcinoma. 5T4 was detected at high levels on carcinomas of bronchi, breast, colon, rectum, stomach, cervix, endometrium, pancreas, ovaria, chorium and seminal vesicles. The cell surface distribution of the 5T4 antigen (e.g., homogeneous or heterogeneous) varied according to tumor type.

To assay cellular localization of the 5T4 antigen, 5T4-expressing cells were cultured as monolayers and then exposed to biotinylated murine H8 antibody. Following cell lysis, membrane-bound 5T4 antigen was separated by avidin binding, and intracellular 5T4 was detected in the non-avidin binding fraction. See FIG. 2.

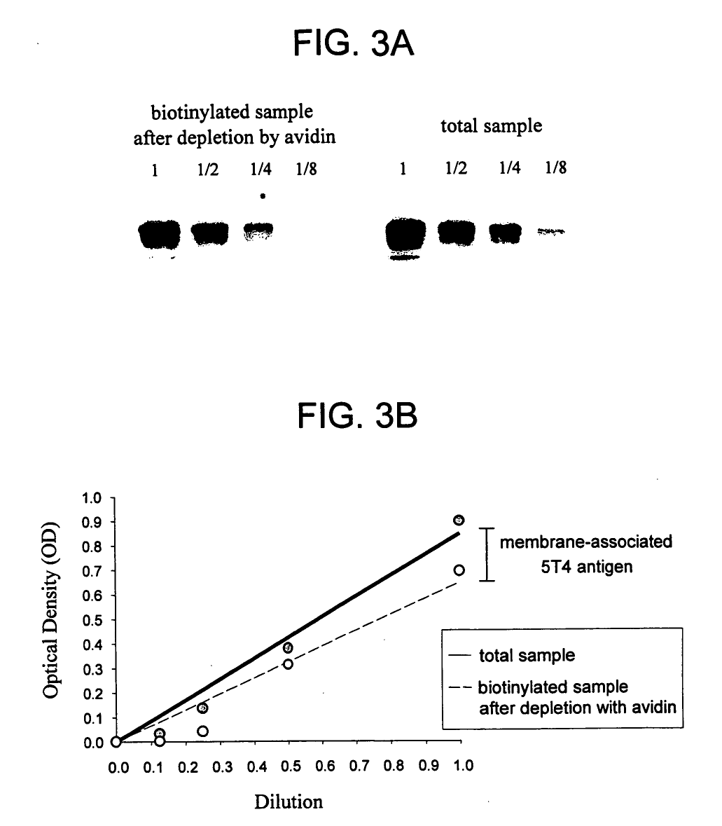

To quantify the percentage of 5T4 antigen present on the cell surface, extracts of biotinylated and control cultures of CT26/5T4 were mixed with avidin-coated beads. Western blot analysis was performed on proteins in the supernatant, and the amount of 5T4 was estimated by densitometry. See FIGS. 3A-3B. Based on the equations of the linear regression lines determined by the dilution of the sample and the optical density of the H8-reactive band, the amount of 5T4 on the cell membrane (5T4M) was calculated using the formula: 100*(1-internal optical density/total optical density). 5T4M was calculated for three cell types: CT26/5T4, 24%; PC3-MM2, 15%, N87, 41%.

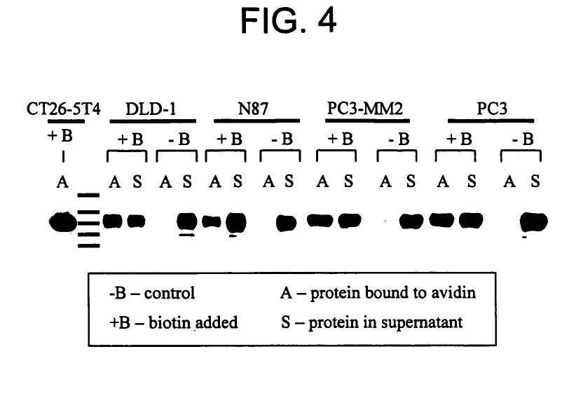

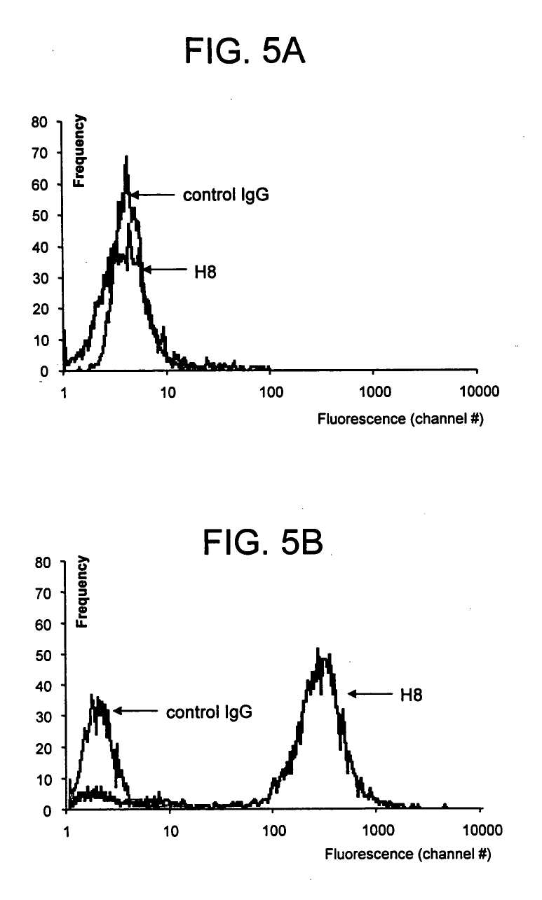

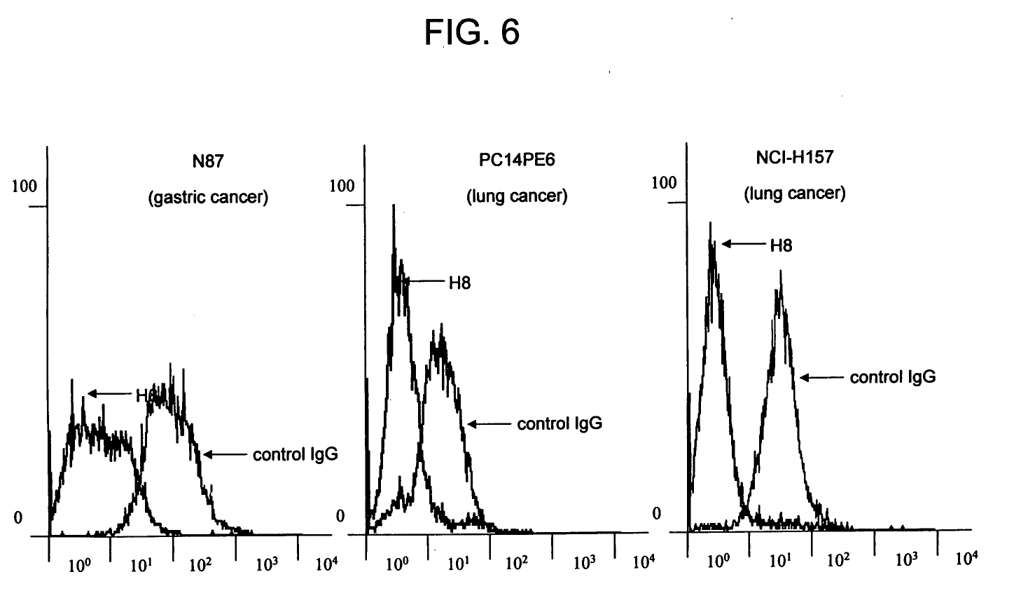

Membrane localization of 5T4 on DLD-1 cells (human colon carcinoma cells), N87 cells (human gastric carcinoma cells), PC3-MM2 cells (human prostrate carcinoma cells), and PC3 cells (human prostrate carcinoma cells) was determined by Western blot analysis following avidin depletion of biotinylated cell cultures. See FIG. 4. Membrane localization of 5T4 on MDAMB435/5T4 cells (human breast cancer cells) was determined by FACS analysis. See FIGS. 5A-5B. Membrane localization of 5T4 on N87, PC14PE6, and NCl-H157 cells was determined by FACS analysis. See FIG. 6. Membrane localization of 5T4 antigen on PC3-MM2 cells was also determined by histochemical detection in tissue samples.

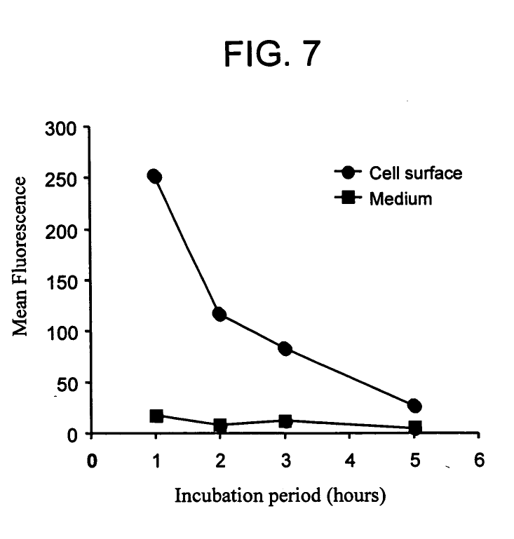

To assess whether the H8 antibody is internalized following binding to 5T4 antigen, the amount of antibody detected at the cell surface was determined over a period of several hours. The disappearance of H8 from the surface of CT26/5T4 cells demonstrates internalization of the 5T4/H8 complex. See FIG. 7.

Table 1 summarizes 5T4 expression in tumor cells. In colorectal carcinoma, gastric carcinoma, and ovarian carcinoma, expression of 5T4 is directly related to progression of the disease. In breast carcinoma, increased intensity of 5T4 staining on metastatic nodules was observed. However, no correlation was found between 5T4 expression in primary tumors and the stage of the disease. See Table 2.

TABLE 1
5T4 Expression in Tumor Cells

| | | | Presence of 5T4 on cell membrane | |
|---|---|---|---|---|
| | | 5T4 Expression | Western blot + | |
| Cell line | Carcinoma | Western blot | biotinylation | FACS |
| DLD-1 | colon | 2/2^{(a)} | 1/1 | 10^{(b)} |
| GEO | colon | 0/2 | n.d. | 4.0 |
| HCT116 | colon | 1/1 | n.d. | 2.2 |
| HT29 | colon | 2/2 | 0/1 | 2.7 |
| LOVO | colon | 0/2 | 0/1 | 3.3 |
| HCA7 | colon | 1/1 | n.d. | 8.0 |
| MDAMB361 | breast | 3/3 | 2/2 | 38.0, 38.0, 34.0 |
| MDAMB435 | breast (melanoma?) | 0/2 | n.d. | 2.4 |
| PC3 | prostate | 4/4 | 1/1 | 3.0 |
| PC3MM2 | prostate | 6/6 | 2/2 | 11.0, 14.0 |
| H157 | lung (NSCLC) | n.d. | 1/1 | 36.3, 41.8 |
| PC14 | lung (NSCLC) | 1/1 | n.d. | 3.5 |
| PC14/PE6 | lung (NSCLC) | 1/1 | n.d. | 3.6 |
| N87 | gastric | 4/4 | 3/3 | 7.6 |
| JAR | chorion | 2/2 | 1/1 | 9.0 |
| JEG | chorion | 2/2 | 1/1 | 2.3 |
| LOX | melanoma | 2/2 | 0/1 | 0.2, 0.9 |
| A431 | cervix | 3/3 | 1/2 | 2.3, 2.3 |
| BXPC3 | pancreas | 1/1 | n.d. | 3.9 |

^{(a)}= fraction of 5T4-positive cultures
^{(b)}= relative mean channel fluorescence = MCF following staining with H8/MCF following staining with mlgG

TABLE 2
5T4 Expression in Breast Carcinoma

| | neoplasia | | | primary anaplasia | | | | | | metastatic |
|---|---|---|---|---|---|---|---|---|---|---|
| | | benign | benign | | | | | grade | | metastatic |
| | | fibrocystic | fibroadenoma | grade 1 | | grade 2 | | 3 | | CA |
| Expression | | 67% (6/9) | 80% (4/5) | 75% (3/4) | | 100% (4/4) | | 89% (8/9) | | 100% (19/19) |
| Homogeneity | | | | | | | | | | |
| 100% | | 33% (3/9) | 60% (3/5) | 75% (3/4) | | 75% (3/4) | | 78% (7/9) | | 68% (13/19) |
| 90% | | | 0% | 0% | | 0% | | 0 | | |
| 75% | | | 0% | 0% | | 0% | | 11% (1/9) | | |
| 50% | {close oversize brace} | 33% (3/9) | 20% (1/5) | 0% | | 25% (1/4) | | 0% | {close oversize brace} | 32% (6/19) |
| 25% | | | 0% | 0% | | 0% | | 0% | | |
| 0% | | 33% (3/9) | 20% (1/5) | 25% (1/4) | | 0% | | 11% (1/9) | | 0% |
| Intensity | | | | | | | | | | |
| 4+ | | 0% | 0% | 0% | | 0% | | 0% | | 0% |
| 3+ | | 11% (1/9) | 0% | 0% | | 25% (1/4) | | | | 47% (9/19) |
| | | | | | | | {close oversize brace} | 22% (2/9) | | |
| 2+ | | | 0% | 25% (1/4) | | | | | | 37% (7/19*) |
| | {close oversize brace} | 56% (5/9) | | | {close oversize brace} | 75% (3/4) | | | | |
| 1+ | | | 80% (4/5) | 50% (2/4) | | | | 67% (6/9) | | 16% (3/19) |

*= 4/19 = 1-2+

### Example 2

### Preparation and Characterization of Anti-5T4—Calicheamicin (CM) Conjugates

The murine H8 antibody was used for preparation of antibody/drug conjugates. The conjugates were then tested in vitro for ability to bind human 5T4 antigen and to induce cytolysis of cancer cells. Three linkers were used to ligate calicheamicin to H8: 4-(4′-acetylphenoxy)butanoic acid (AcBut), 3-acetylphenyl acidic acid (AcPac) and 4-mercapto-4-methyl-pentanoic acid (Amide). To increase the amount of calicheamicin in H8-calicheamicin conjugates, the antibody was conjugated to PEG prior to conjugation with calicheamicin, for example, using PEG-SPA, PEG-SBA, and PEG-bis-maleimide. In Table 4, the efficiency of each of the H8-calicheamicin conjugates is reported as ED50, which is the amount of calicheamicin given as conjugate or as free drug that caused 50% reduction of a cell culture relative to an untreated control. The number of cells was determined using a vital dye (MTS).

Direct linkage of calicheamicin to H8 via the AcBut (4-(4′-acetylphenoxy)butanoic acid) acid-hydrolyzable linker proved difficult and generated mostly aggregated conjugate and conjugate with low amounts of CM per antibody (approximately 1 mole/mole). The low level of conjugation and aggregation properties of the resultant complexes rendered these complexes unsuitable for use.

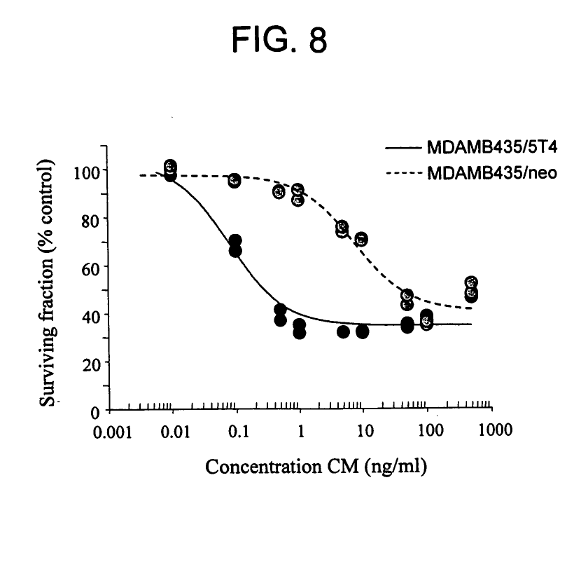

Linkage of calicheamicin to H8 via a stable amide linker (4-mercapto-4-methyl-pentanoic acid) resulted in a conjugate that was 200-fold more cytotoxic against a 5T4 expressing carcinoma when compared to its 5T4-negative counterpart. See FIG. 8 and Table 4. Despite this selectivity, the conjugate was less cytotoxic than free calicheamicin and was unable to completely destroy the cell culture at concentrations of 500 ng calicheamicin per ml of medium.

Conjugates prepared using polyethylene glycol (PEG), H8, calicheamicin, and the AcBut linker did not form aggregates and had a loading of approximately 6 mole calicheamicin per mole H8. Multiple types of PEG tested reduced the binding of H8 to 5T4 by 50% to 90%. See Table 3.

TABLE 3
Binding Activity of H8 Antibody/PEG

| | | Binding of H8PEG* | |
|---|---|---|---|
| Name | MW (kDa) | BIACORE ® | FACS |
| PEG-SPA (PEG2K) | 2 | 69% | 64% |
| PEG-SBA (PEG5K) | 5.3 | 79% | 79% |
| PEG-bis-maleimide | 3.4 | 43% | not |
| (PEGmal2) | | | determined |

*fraction of binding to 5T4 lost following antibody modification

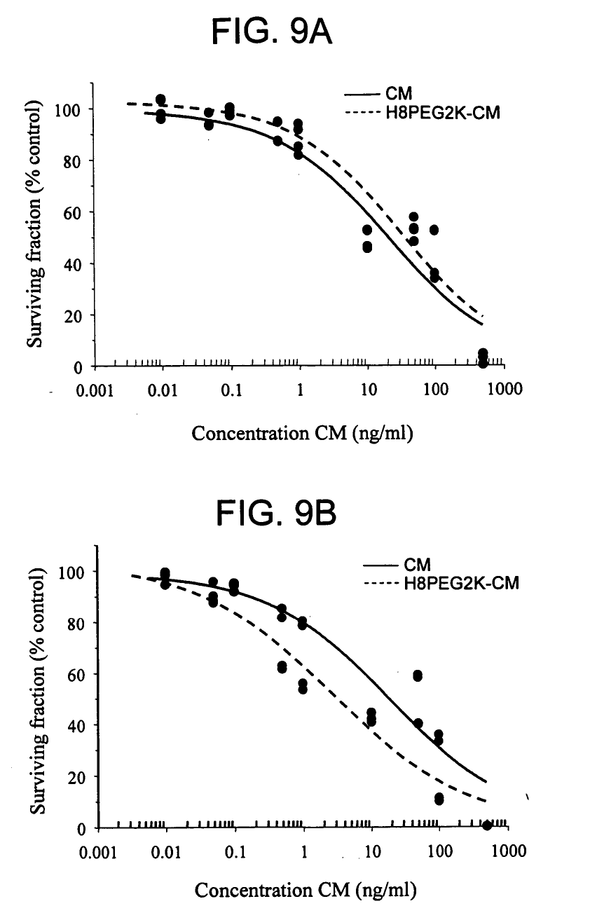

Despite reduced binding, two H8PEG-AcBut-CalichDMH conjugates, H8PEG2K-AcBut-CalichDMH and H8PEGmal2-AcBut-CalichDMH, proved selectively efficacious for inducing cytolysis of 5T4-expressing cells in vitro. Selective cytotoxicity was observed for H8-calicheamicin conjugates that retained only about 30% of binding activity. H8PEG2K-AcBut-CalichDMH was also more cytotoxic than free calicheamicin and completely destroyed the cell culture at concentrations of 500 ng calicheamicin per ml of medium. See FIGS. 9A-9B and Table 4.

TABLE 4
Selective Cytolysis of 5T4-Expressing Cells by H8-Calicheamicin Conjugates

| | | | | Cell lines | | | | | |
|---|---|---|---|---|---|---|---|---|---|
| | | | | MDAMB435 | | MDAMB435/neo | | | |
| Treatment | | | | ED₅₀ | range | ED₅₀ | | MDAMB435/5T4 | |
| name | antibody | PEG | linker | (ng/ml) | (ng/ml) | (ng/ml) | range (ng-ml) | ED₅₀ (ng/ml) | range (ng-ml) |
| CM | none | none | none | 3.80 (4)^{a} | 0.70-7.00 | 11.90 (7) | 2.00-30.00 | 10.50 (6) | 5.00-20.00 |
| CMA | p67.6 | none | AcBut | 41.00 (3) | 23.00-70.00 | 50.00 (5) | 20.00-90.00 | 55.00 (4) | 20.00-80.00 |
| H8-CM | H8 | none | AcBut | 3.00 (1) | 3.00-3.00 | 2.30 (1) | 2.30-2.30 | 0.04 (1) | 0.04-0.04 |
| H8˜CM | H8 | none | AcPac | n.d. | n.d. | 1.00 (1) | 1.00-1.00 | 0.02 (1) | 0.02-0.02 |
| H8\hat{ }CM | H8 | none | Amide | n.d. | n.d. | 40.00 (1) | 40.00-40.00 | 0.18 (1) | 0.18-0.18 |
| H8PEG2K-CM | H8 | PEG2K | AcBut | 31.00 (2) | 12.00-50.00 | 41.50 (2) | 23.00-60.00 | 2.60 (2) | 2.00-3.20 |
| H8PEG5K-CM | H8 | PEG5K | AcBut | 36.00 (2) | 22.00-50.00 | 45.00 (2) | 40.00-50.00 | 31.50 (2) | 30.00-33.00 |
| H8PEGmal2-CM | H8 | PEGmal2 | AcBut | n.d. | n.d. | 7.00 (1) | 7.00-7.00 | 0.90 (1) | 0.90-0.90 |

Numbers in parentheses indicate the number of experiments.

### Example 3

### Anti-Tumor Efficacy of H8-Calicheamicin Conjugates Using Subcutaneous Xenografts

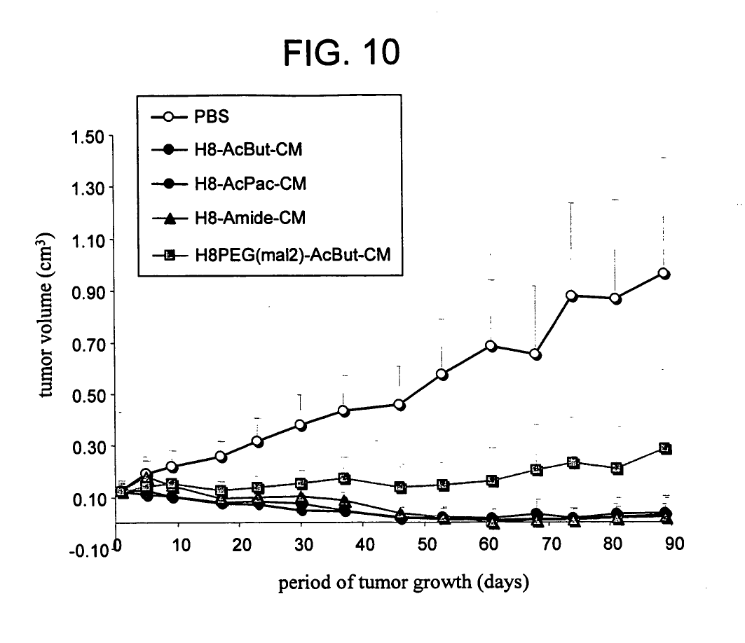

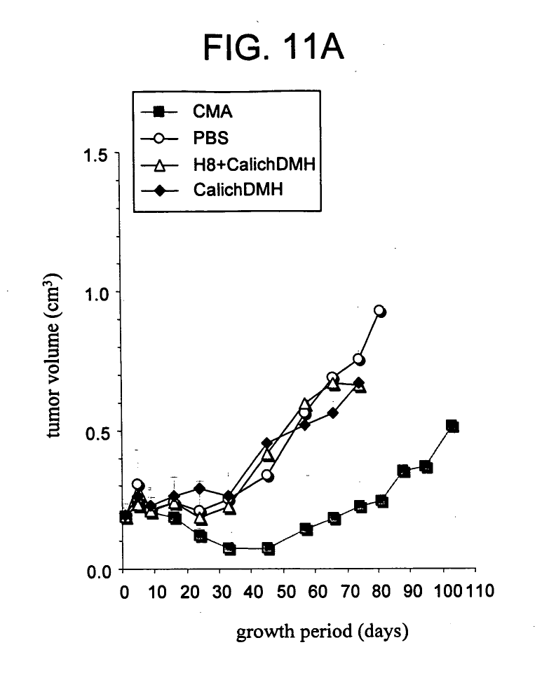

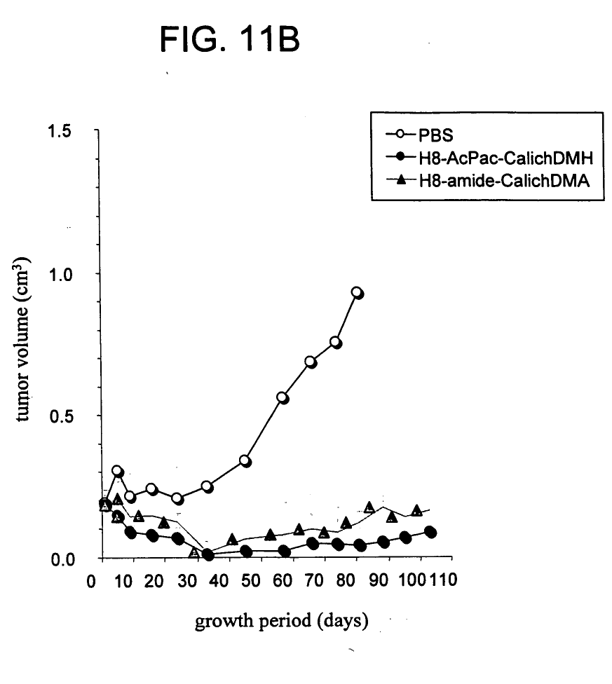

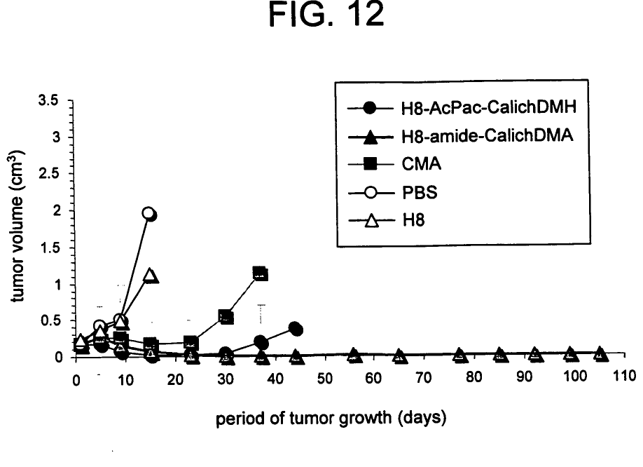

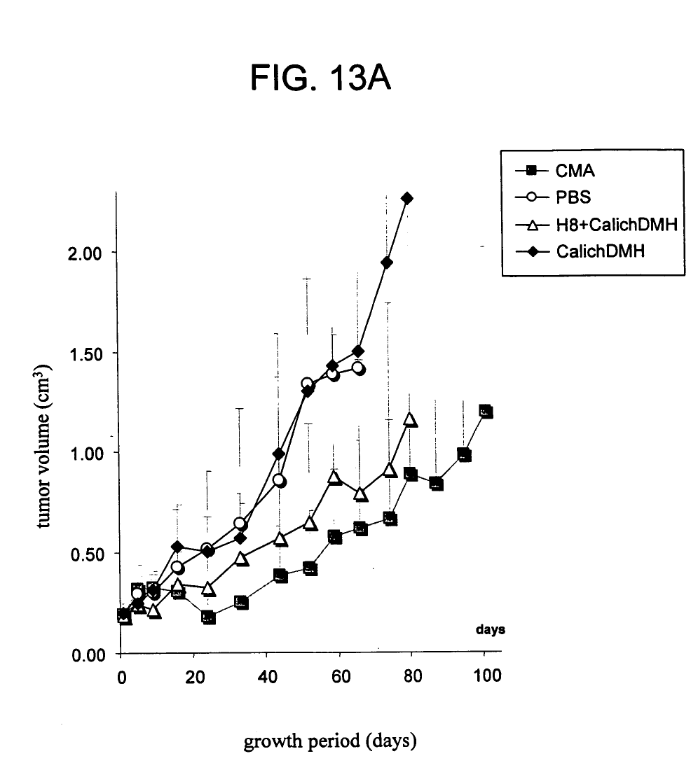

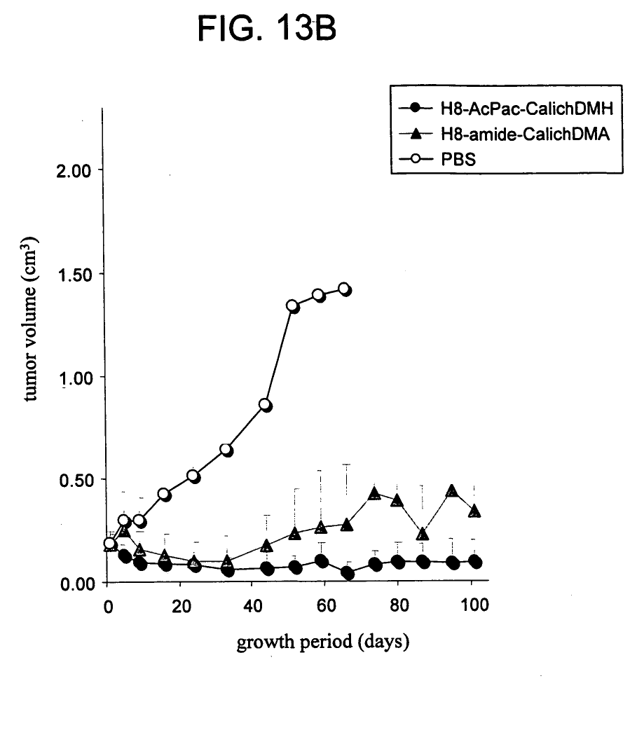

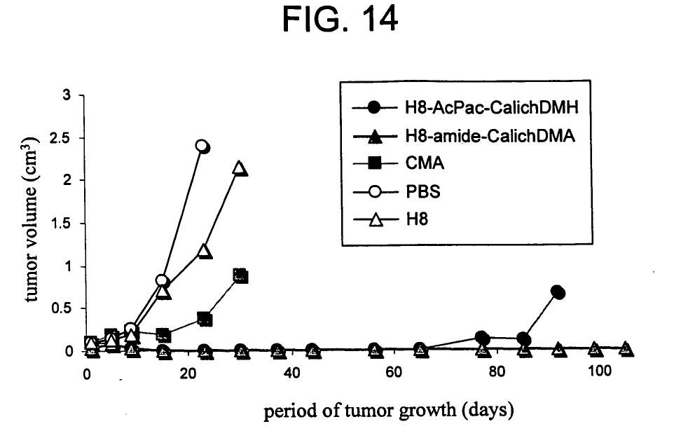

To assess the cytotoxicity of H8-calicheamicin conjugates in vivo, tumors were prepared in nude mice by subcutaneous injection of MDAMB435/5T4 cells (human breast carcinoma cells overexpressing human 5T4 antigen), NCl—H157 cells (human non-small cell lung cancer cells), PC14PE6 cells (human non-small cell lung cancer cells), or N87 cells (human gastric carcinoma cells). H8-calicheacmicin conjugates and control compounds were administered by intraperitoneal injection to tumor-bearing mice in a total of 3 doses given at 4-day intervals, i.e., on days 1, 5, and 9. H8-calicheamicin conjugates inhibited growth of all tumor types. See FIGS. 10, 11A-11B, 12, 13A-13B, and 14.

### Example 4

### Anti-Tumor Efficacy of H8-Calicheamicin Conjugates Using an Orthotopic Model of Human Lung Cancer

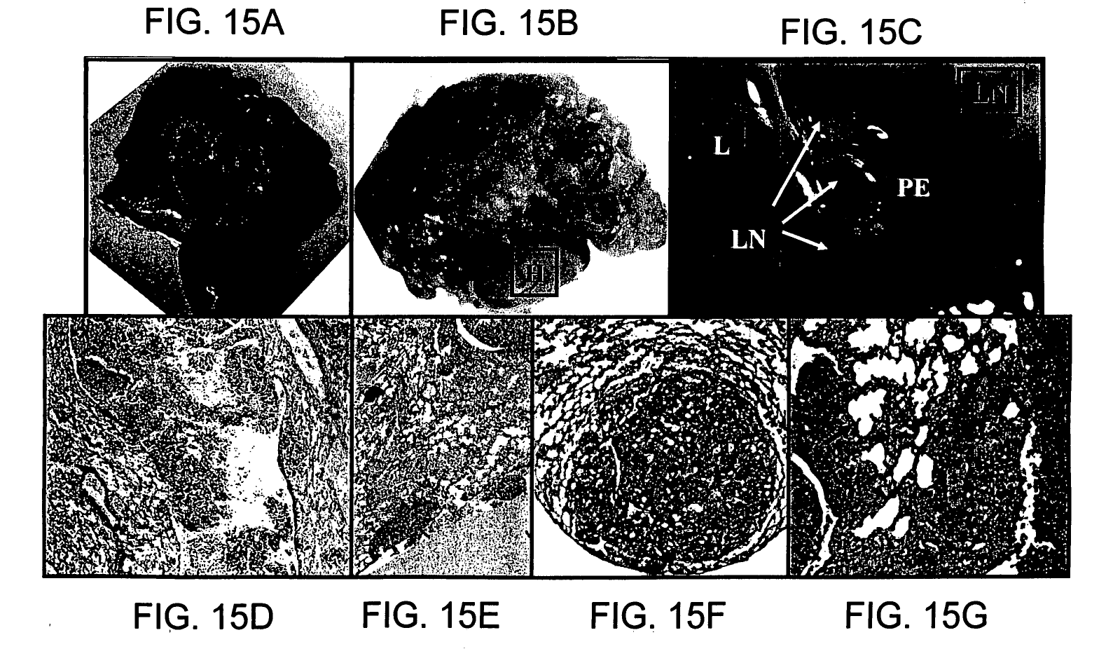

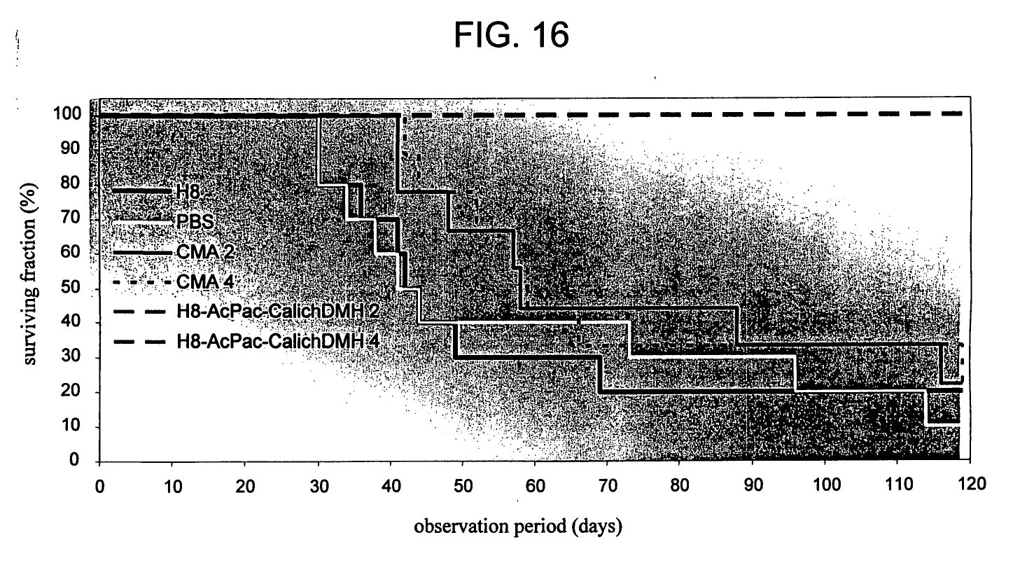

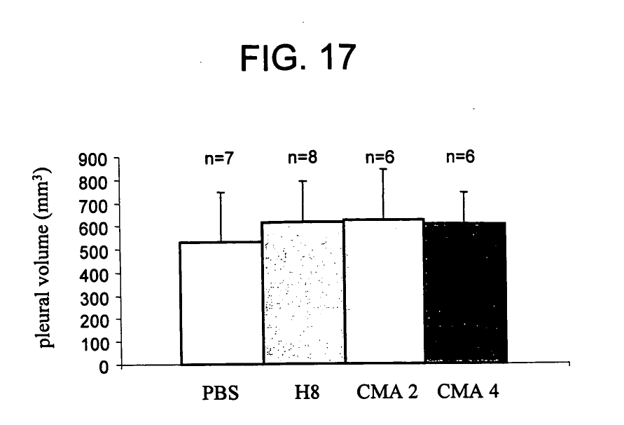

To further assess the targeting ability of antibodies having an H8-calicheamicin conjugates, an orthotopic model for non-small cell and small cell cancer was used, essentially as described by Onn et al. (2003) Clin. Cancer Res. 9(15):5532-5539. In brief, human lung adenocarcinoma (PC14PE6) cells were injected into tail veins of nude mice, which then migrated to form tumors in lung. Tumors appeared as solid nodules in the lung parenchyma and caused hemorrhagic pleural effusions containing suspended tumor cells. See FIGS. 15A-15G. Injection of control compounds and H8-calicheacmicin conjugates were administered by intraperitoneal injection to tumor-bearing mice beginning at 6 days after injection of tumor cells for a total of 3 doses given at 4-day intervals, i.e., on days 6, 10, and 14. Administration of H8-calicheacmicin conjugates resulted in increased survival of tumor-bearing animals. See FIG. 16. Administration of unconjugated H8 antibody or the control conjugate CMA did not reduce the pleural effusions. However, CMS slightly increased the average survival of the tumor-bearing mice. See FIG. 17.

### Example 5

### Preparation and Characterization of Humanized Anti-5T4 Antibodies

Chimeric and humanized anti-5T4 antibodies were prepared using sequences derived from the murine H8 antibody and human antibody sequences. The sequences of representative antibodies of the invention are shown in FIGS. 27A-27F.

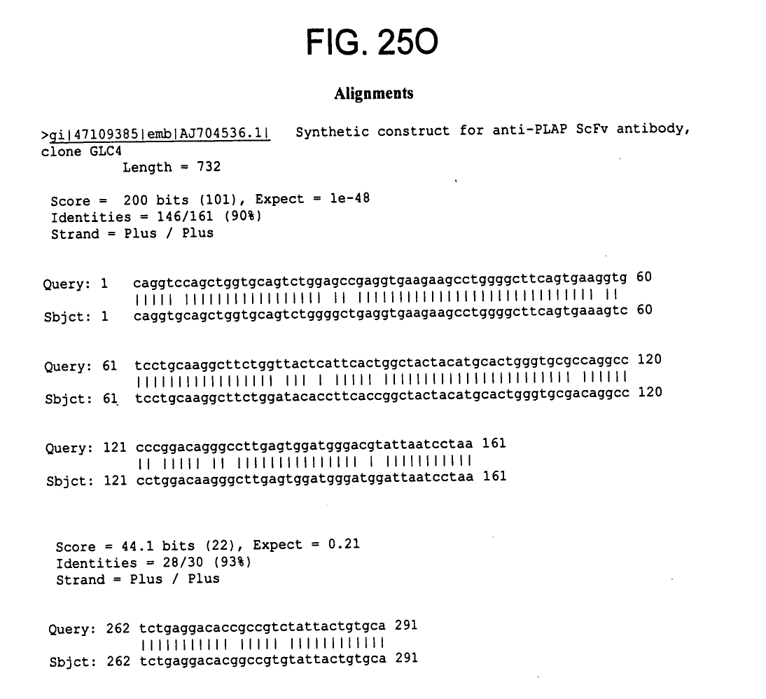

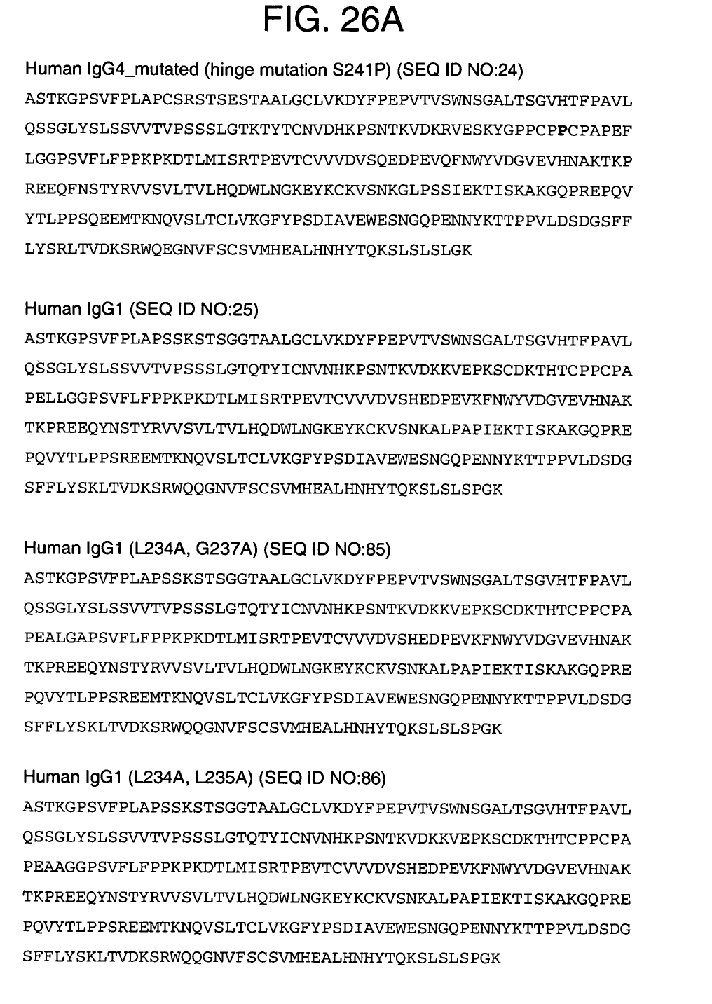

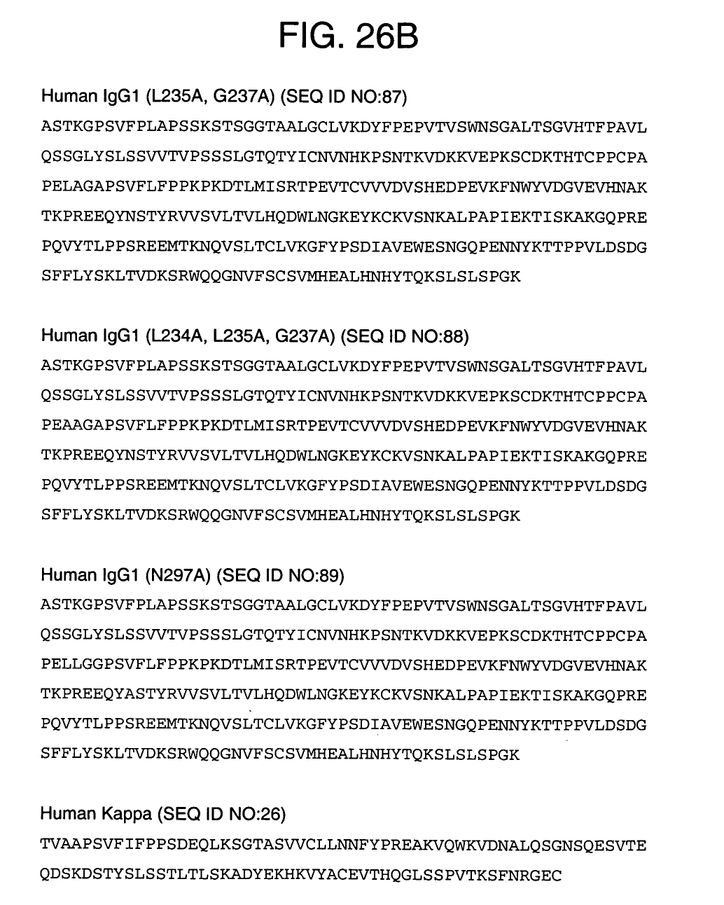

Chimeric H8 antibodies were constructed having murine H8 heavy chain and light chain variable regions sequences and human constant regions sequences (FIGS. 27A-27B). Representative human constant regions that were used to prepare chimeric and humanized H8 antibodies include those of human IgG1, human kappa, and human IgG4. Mutations were optionally introduced to alter constant region effector functions, such as cellular dependent cytotoxicity (CDC), complement lysis, and antibody dependent cellular cytotoxicity (ADCC). See FIGS. 26A-26B. For cloning of sequences encoding IgG constant regions, intronic sequences may optionally be deleted. See Example 6. Antibodies were also prepared wherein one antibody chain comprises the murine H8 variable region (as in a chimeric antibody) and the other antibody chain comprises a humanized H8 variable region, i.e., a semi-humanized antibody (FIG. 27C).

Humanized H8 variable regions were constructed to include the CDRs of murine H8 grafted onto human or substantially human framework regions. The CDRs of the murine H8 antibody were identified using the AbM definition, which is based on sequence variability as well as the location of the structural loop regions. Human acceptor frameworks were selected on the basis that they were substantially similar to the framework regions of the murine H8 antibody, or which were most similar to the consensus sequence of the variable region subfamily. See FIGS. 18-23. Consideration was also given to representation of the framework loci in humans, such that widely represented sequences were preferred over less populous sequences. Additional mutations of the human framework acceptor sequences were made, for example to restore murine residues believed to be involved in antigen contacts and/or residues involved in the structural integrity of the antigen-binding site. The amino acid sequence was also optimized for codon preference of CHO cells and to remove restriction enzyme sites. A peptide structure prediction program was used to analyze the humanized variable heavy and light region sequences to identify and avoid post-translational protein modification sites introduced by the humanization design. Using this strategy, three versions of humanized H8 variable regions were constructed. Version 1 retains murine H8 residues at positions within the framework sequence believed to be critical for antibody integrity and antigen binding. Version 2 retains murine residues only in the CDRs. Version 3 is similar to version 2, with the exception that a consensus variable region sequence was used as the heavy chain acceptor framework. The light chain variable region of the version 3 antibody is the same as that of the Version 2 antibody. See FIGS. 24A-24C.

The H8 anti-5T4 antibody variable heavy and light regions were cloned using the SMART® cDNA synthesis (Clontech) followed by PCR amplification. The cDNA was synthesized from 1 μg total RNA isolated from the H8 hybridoma cells, using oligo(dT) and the SMART® IIA oligo with POWERSCRIPT™ reverse transcriptase (Clontech). The cDNA was then amplified by PCR using a primer which anneals to the SMART® IIA oligo sequence and a human constant region specific primer (mouse Kappa for the light chain, mouse IgG1 for the heavy chain) with VENT® polymerase. Heavy and light chain PCR products were subcloned into the pED6 expression vector and the nucleic acid sequence was determined. This method is advantageous in that no prior knowledge of the DNA sequence is required. In addition, the resultant DNA sequence is not altered by use of degenerate PCR primers.

Several discrepancies were noted between the nucleotide sequence of murine H8 when compared to the published nucleotide sequence (PCT International Publication No. WO 98/55607 and in Forsberg et al. (1997) J. Biol. Chem. 272(19):124430-12436). The noted differences do not alter the protein sequence. The T (published) to C difference present in the codon for amino acid 133 of the heavy chain variable region correlates with the codons ACT and ACC, respectively. Both ACT and ACC code for the amino acid threonine (T). The T (published) to C difference present in the codon for amino acid 138 of the heavy chain variable region correlates with the codons TCT and TCC, respectively. Both TCT and TCC code for the amino acid serine (S). The A (published) to C difference present in the codon for amino acid 126 of the light chain variable region correlates with the codons ATA and ATC, respectively. Both ATA and ATC code for the amino acid isoleucine (I).

For construction of humanized H8 light chain variable regions, the DPK24 germ line sequence VL-IV/locus B3 was used as the acceptor framework. The DPK24 sequence is 68% identical to the murine H8 light chain variable region and contains 18 amino acid substitutions when compared to the murine H8 light chain framework sequences. Humanized H8 light chain variable region version 1 maintained murine H8 residues S43, S49, and F87. Mutations which were determined to increase expression include F10S, T45K, 163S, Y67S, F73L, and T77S. See FIG. 18.

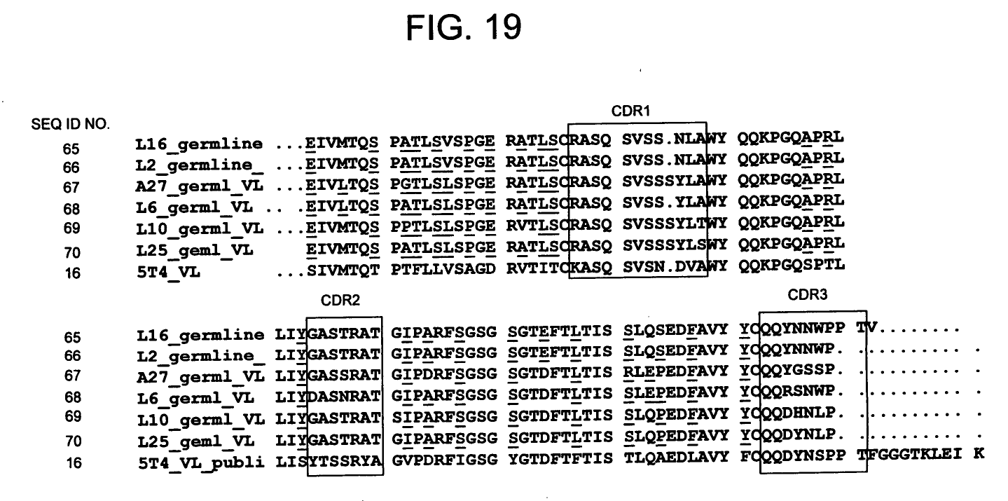

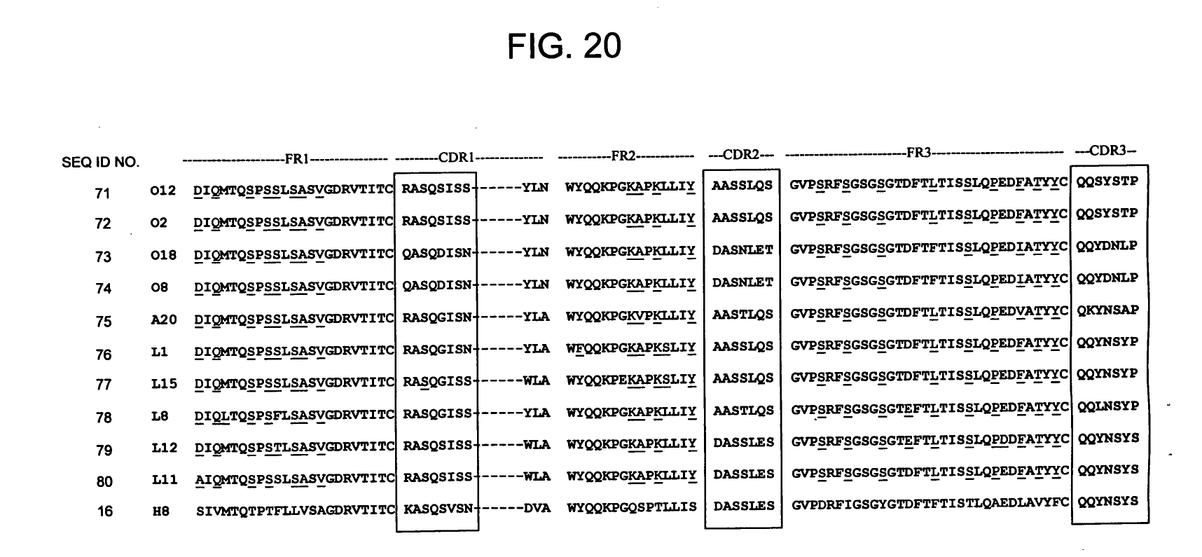

Humanized antibodies were also constructed using framework regions of the light chain variable region of germline clone subgroups VκIII and VκI. See FIGS. 19-20. In particular, antibodies that include light chain VκIII subgroups framework regions and the disclosed humanized H8 antibody version 1 are both highly expressed and stable. Nine mutations (T7S, D17E, V19A, S50Y, I63S, Y67S, F73L, T77S, and F87Y) for humanization of the H8 light chain variable region based on the VκIII germline frameworks L16, L2, A27, L6, L10, and L25 have been introduced in humanized H8 antibody version 2 and do not compromise binding affinity. Similarly, ten mutations (T7S, F10S, V19A, T46K, S50Y, I63S, T67S, F73L, T77S, and F87Y) for humanization of the H8 light chain variable region based on the germline frameworks from subgroup VκI have been introduced in humanized H8 antibody without affecting binding affinity. In addition, substitutions of amino acids at positions 1, 9, 10, 12, 15, 22, 43, 45, and 83 of the H8 light chain variable region do not affect antigen binding.

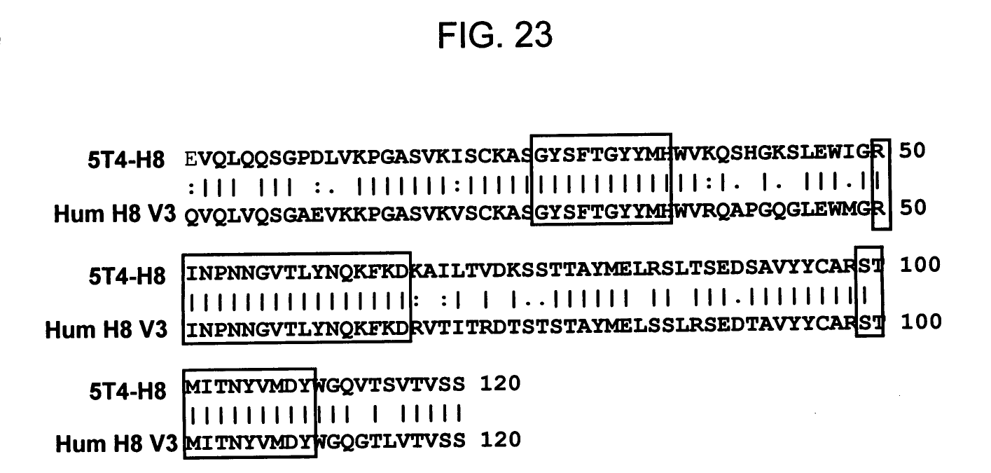

For construction of humanized H8 heavy chain variable regions, the DP75 germ line sequence VH-I/locus 1-02 was used as the acceptor sequence. The DP75 sequence is 65% identical to the murine H8 heavy chain variable region and contains 28 amino acid substitutions when compared to the murine H8 heavy chain framework sequence. Humanized H8 heavy chain variable region version 1 maintained murine H8 residues K38 and S40, which are important for antigen contact with the heavy chain and light chain variable regions, as well as I48, which is important for antigen contact with the variable regions and with CDR2. See FIG. 21. Alternatively, humanized H8 heavy chain version 3 was prepared using a heavy chain variable region subgroup consensus sequence. The consensus sequence contains 25 amino acid substitutions when compared to the murine H8 heavy chain framework sequence. See FIGS. 22-23.

The humanized H8 heavy chain and light chain variable regions were constructed by annealing together overlapping oligonucleotides and ligating them into the pED6 expression vector containing a human antibody constant region. Humanized heavy chain and light chain variable regions may also be constructed using PCR mutagenesis or site-directed mutagenesis. Design of the oligonucleotides included optimization of codon usage for CHO cell expression and removal of restriction enzyme sites. A Bgl II restriction site was removed from the H8 variable heavy region. Oligonucleotides used to synthesize humanized H8 light chain variable region version 1 are set forth as SEQ ID NOs:27-32. Oligonucleotides used to synthesize humanized H8 light chain variable region version 2 are set forth as SEQ ID NOs:33-36. Oligonucleotides used to synthesize humanized H8 heavy chain variable region version 1 are set forth as SEQ ID NOs:37-44. Oligonucleotides used to synthesize humanized H8 heavy chain variable region version 2 are set forth as SEQ ID NOs:37, 39-42, and 44-46. Oligonucleotides used to synthesize humanized H8 heavy chain variable region version 3 are set forth as SEQ ID NOs:37, 40, 41, and 44-48.

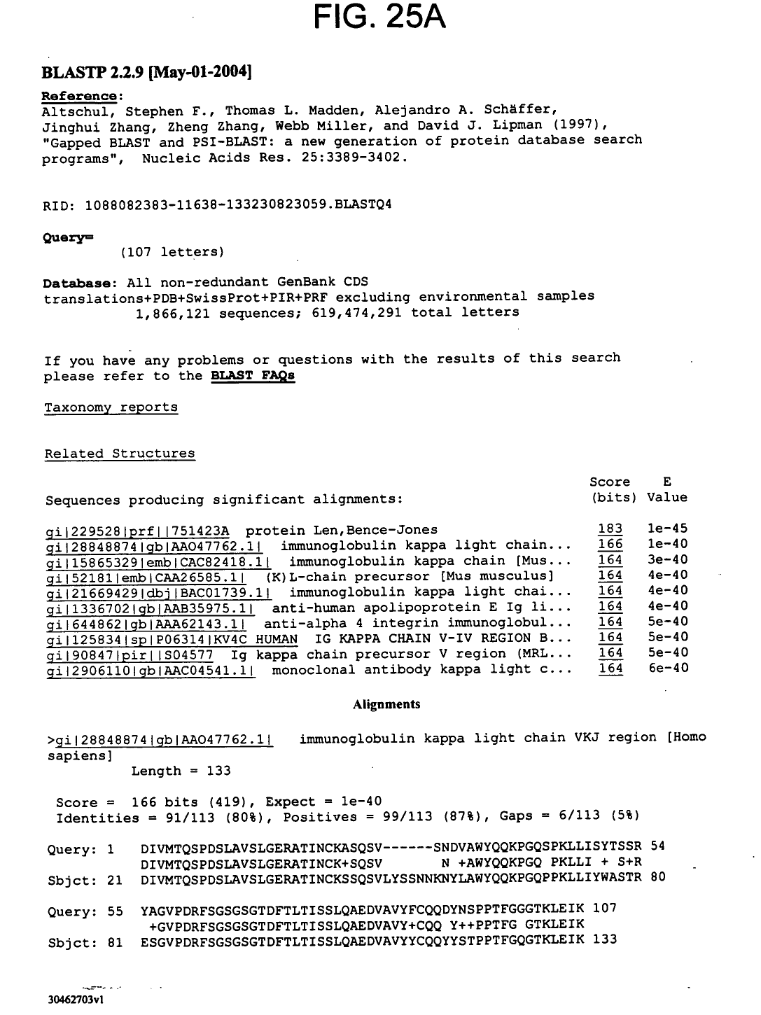

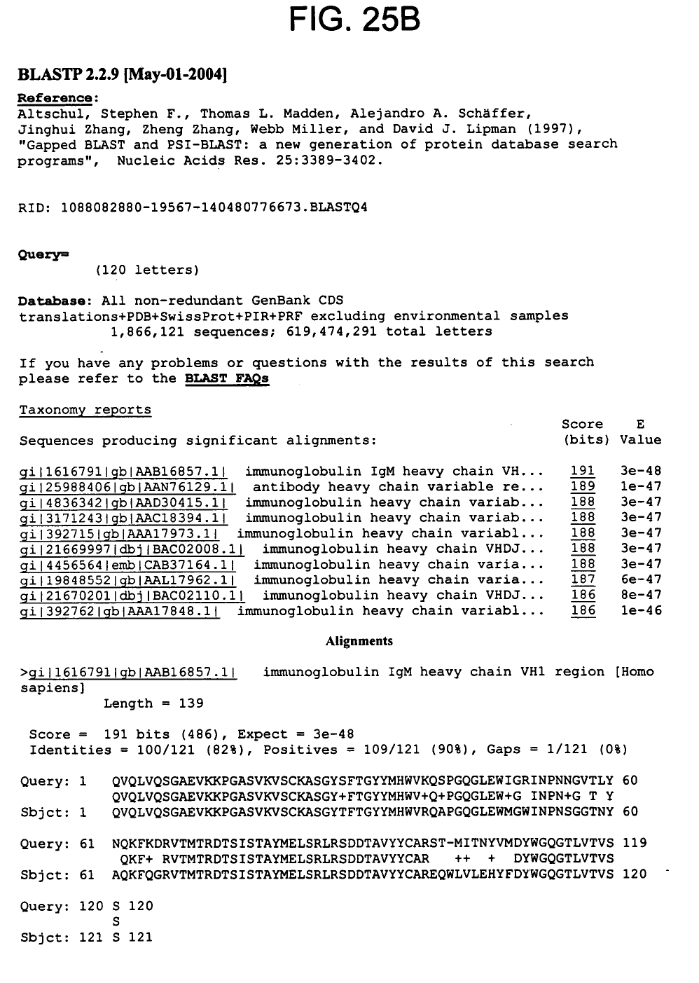

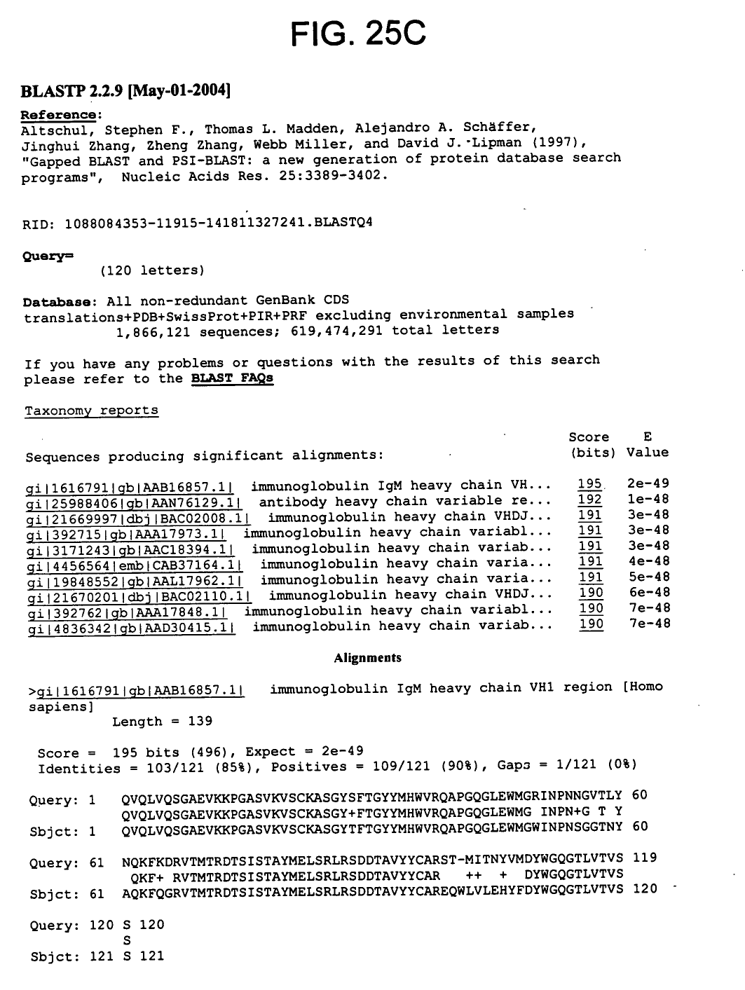

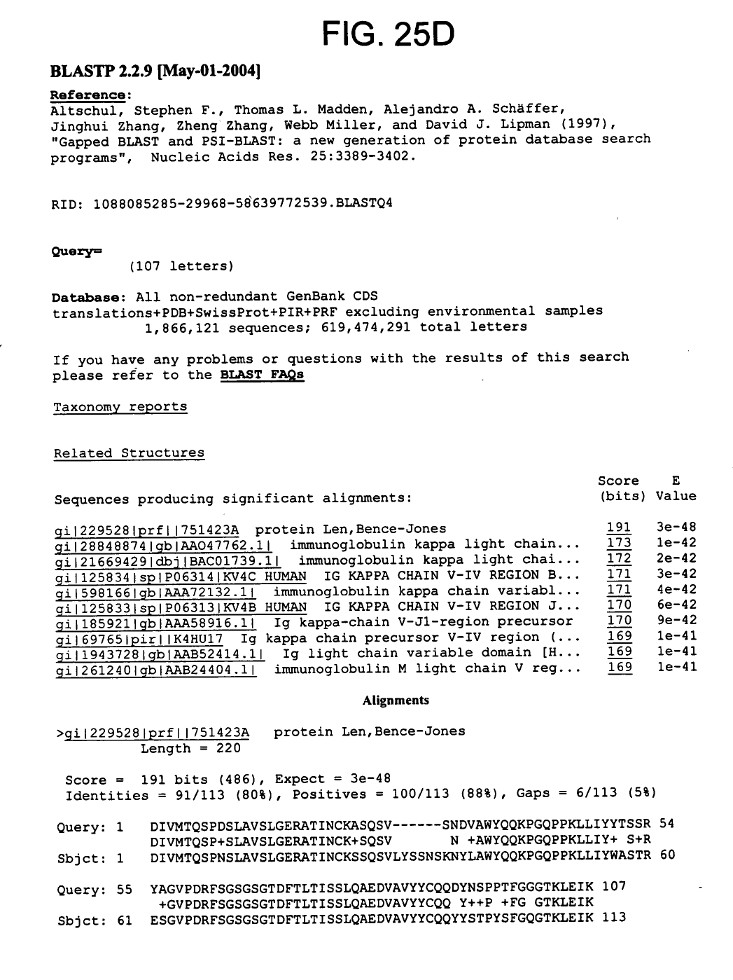

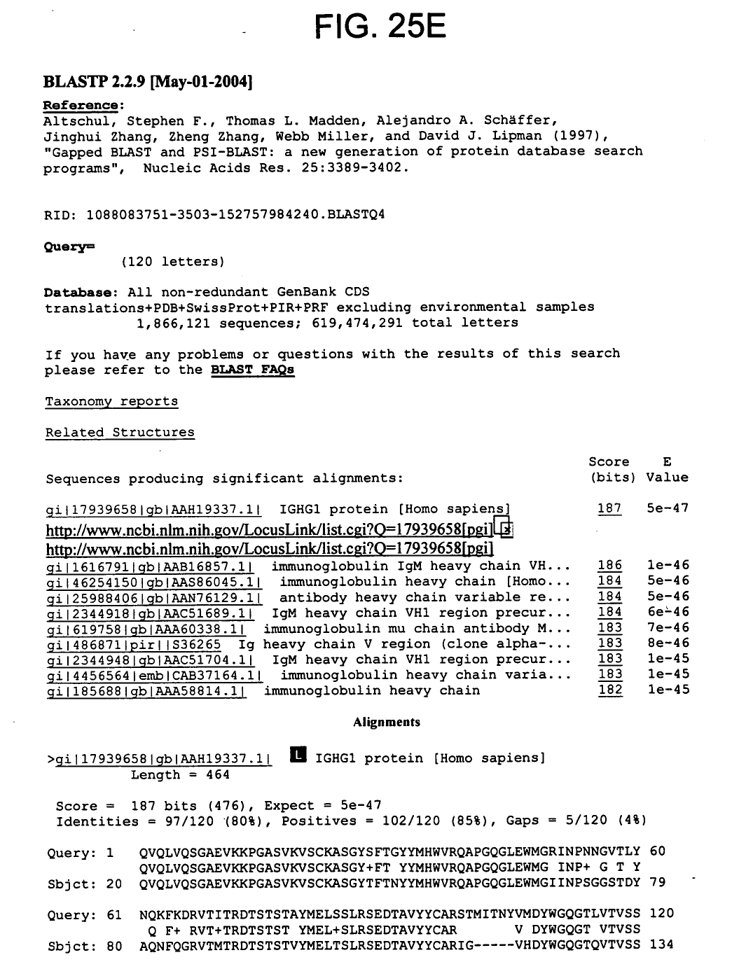

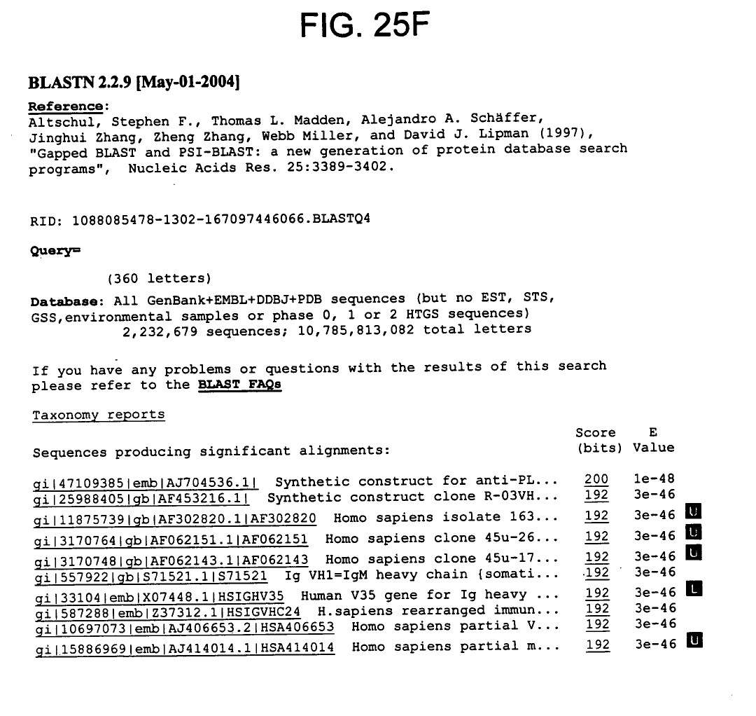

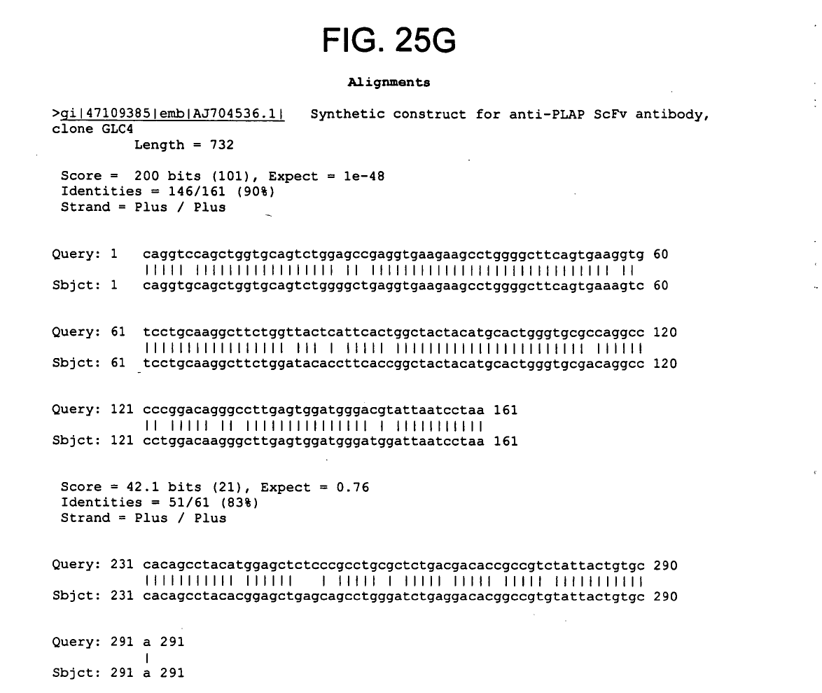

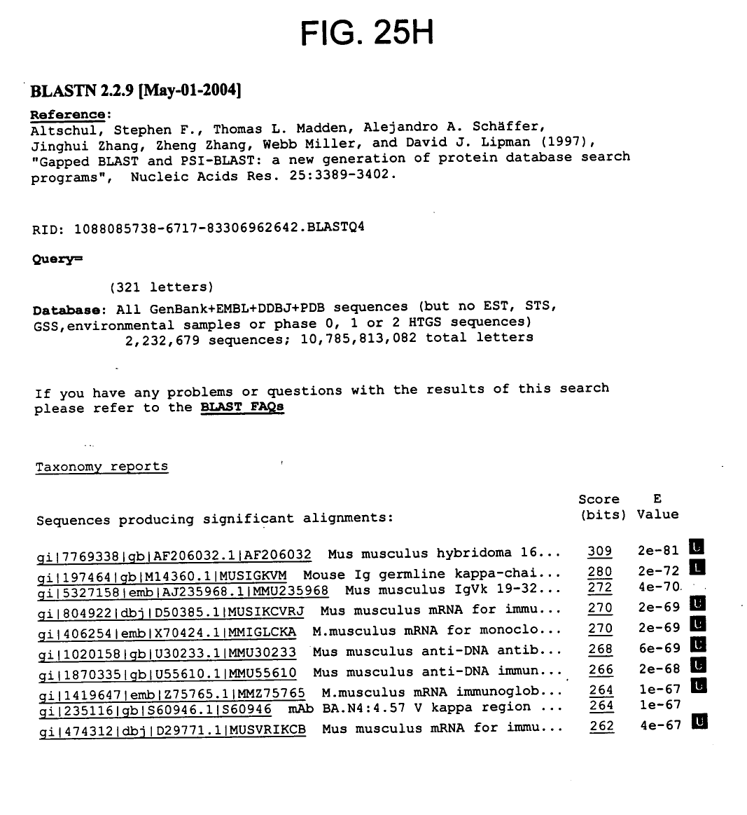

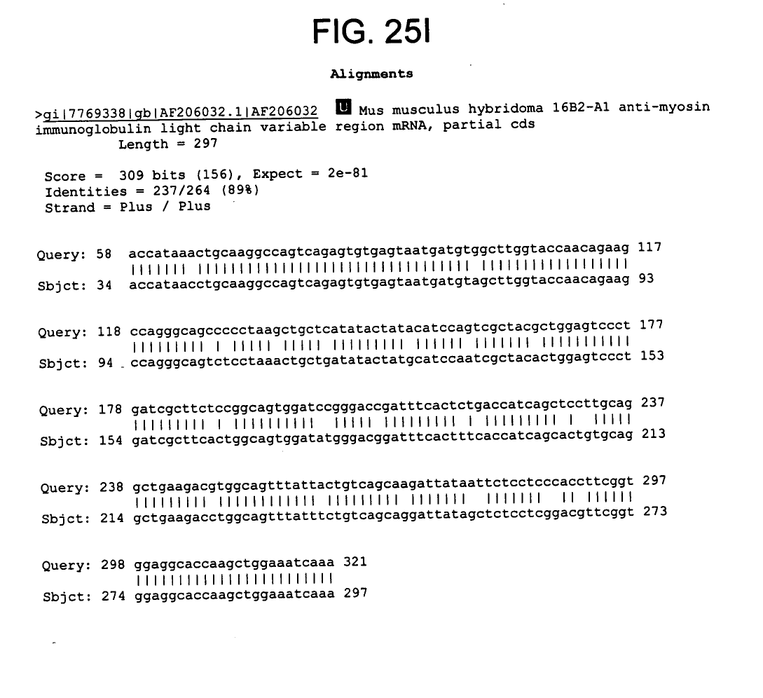

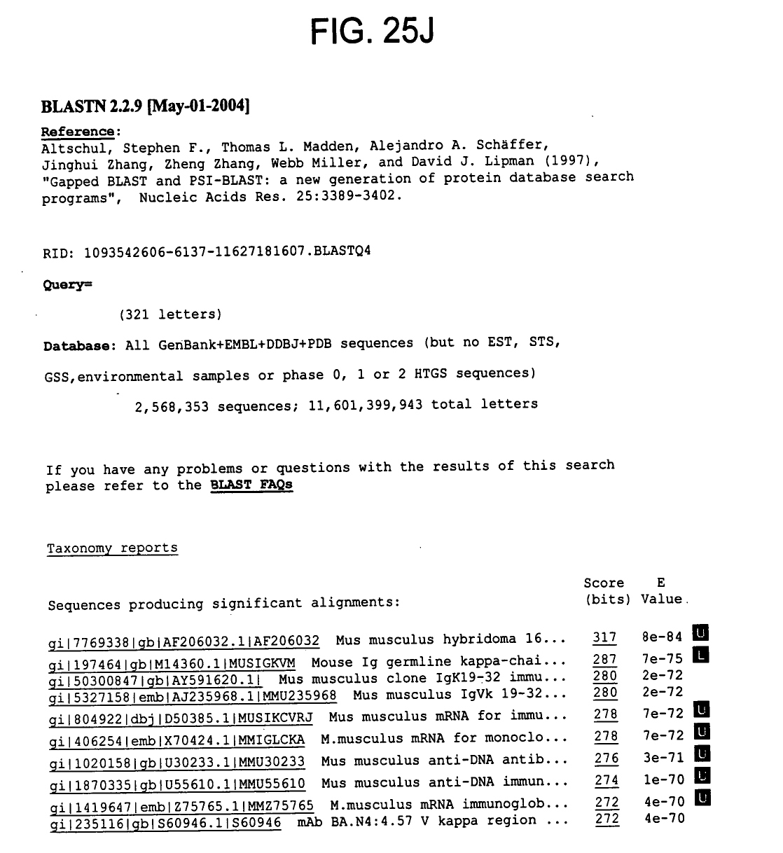

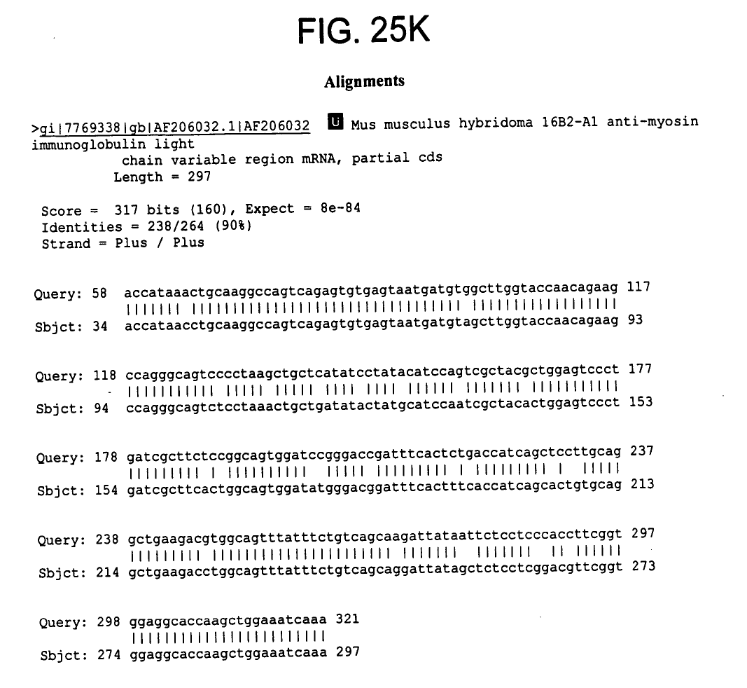

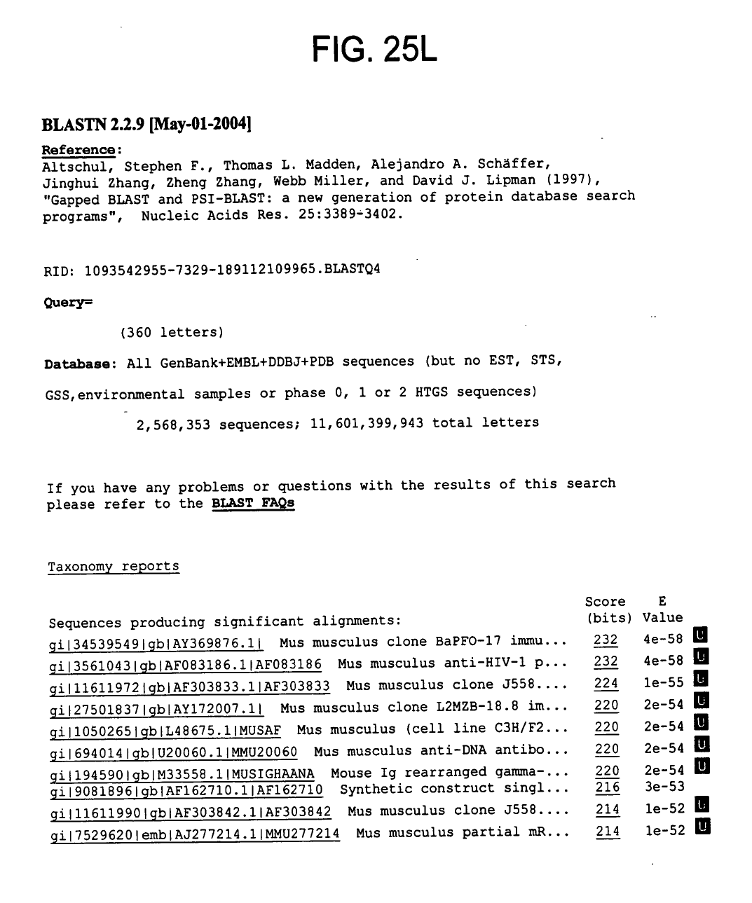

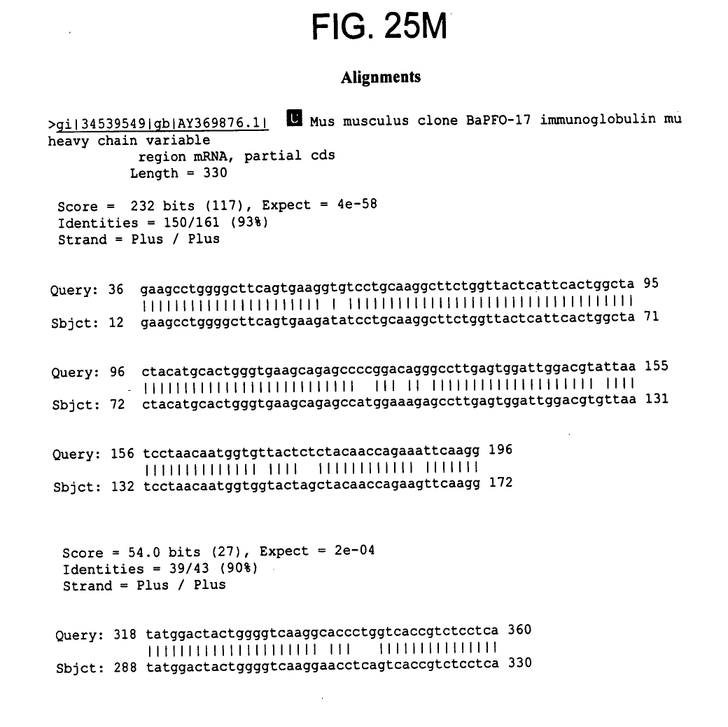

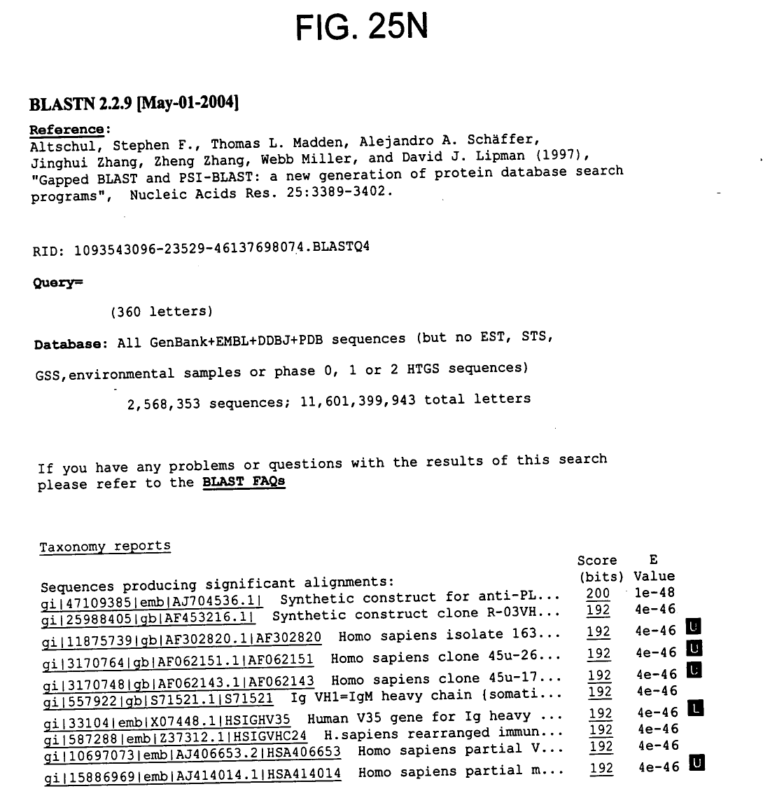

To assess the novelty of the humanized H8 variable region sequences, BLASTp searches (for protein query sequences) were conducted using default parameters of Expect=10, Word Size=3, a low complexity filter, and the BLOSUM62 matrix, permitting gap costs of existence=11, and extension=1. BLASTn searches (for nucleotide query sequences) were conducted using default parameters of Expect=10, Word Size=11, and a low complexity filter. BLAST search results are reported as a list of sequences related to the query sequence, ranked in order of E value, which is an indicator of the statistical significance of matches identified in the database. Sequences most closely related to the humanized variable region sequences used for BLAST analysis are identified in Table 5 (BLASTp) and Table 6 (BLASTn). The BLAST results and an alignment of each query sequence with the most closely related subject sequence are shown in FIGS. 25A-25O.

TABLE 5
BLASTp Analysis

| | Identity | | |
|---|---|---|---|
| | (%) of | | |
| | Closest | Description of | |
| Query | Subject | Closest Subject | Fig- |
| Sequence | Sequence | Sequence | ure |
| VL version 1 | 77% | gi\|229528\|prf\|751423A | 25A |
| (SEQ ID | | protein Len, Bence-Jones | |
| NO:17) | | | |
| VL version 2 | 80% | gi\|229528\|prf\|751423A | 25C |
| (SEQ ID | | protein Len, Bence-Jones | |
| NO:23) | | | |
| VH version 1 | 82% | gi\|161791\|gb\|AABl6857.1 | 25B |
| (SEQ ID | | Homo sapiens | |
| NO:18) | | immunoglobulin IgM | |
| | | heavy chain VH1 region | |
| VH version 2 | 85% | gi\|161791\|gb\|AAB16857.1 | 25D |
| (SEQ ID | | Homo sapiens | |
| NO:21) | | immunoglobulin IgM | |
| | | heavy chain VH1 region | |
| VH version 3 | 80% | gi\|17939658\|gb\|AAH19337.1\| | 25E |
| (SEQ ID | | Homo sapiens | |
| NO:19) | | IGHG1 protein | |

TABLE 6
BLASTn Analysis

| | Identity (%) of | Description of | |
|---|---|---|---|
| | Closest Subject | Closest Subject | |
| Query Sequence | Sequence | Sequence | Figure |
| VL version 1 | 90% | gi\|7769338\|gb\|AF2O6032.1 | 25J-25K |
| (SEQ ID NO:81) | | Mus musculus hybridoma 16B2-A1 anti- | |
| | | myosin immunoglobulin light chain | |
| | | variable region mRNA, partial cds | |
| VL version 2 | 89% | gi\|7769338\|gb\|AF206032.1 | 25F-25G |
| (SEQ ID NO:22) | | Mus musculus hybridoma 16B2-A1 | |
| | | anti-myosin immunoglobulin light chain | |
| | | variable region mRNA, partial cds | |
| VH version 1 | 93% | gi\|34539549\|gb\|AY369876.1 | 25L-25M |
| (SEQ ID NO:82) | | Mus musculus clone BaPFO-17 | |
| | | immunoglobulin mu heavy chain | |
| | | variable region mRNA, partial cds | |
| VH version 2 | | gi\|47109385\|emb\|AJ7045366.1 | |
| (SEQ ID NO:20) | | synthetic construct for anti-PLAP ScFv | 25H-25I |
| | | antibody, clone GLC4 | |
| VH version | | gi\|47109385\|emb\|AJ704536.1\| | 25N-250 |
| (SEQ ID NO:83) | | Synthetic construct for anti-PLAP ScFv | |
| | | antibody, clone GLC4 | |

To confirm that the chimeric and humanized antibodies could be expressed, COS-1 cells were transiently transfected with plasmids encoding representative anti-5T4 antibodies of the invention. After a period of 48 hours, the cell culture medium was assayed to determine levels of human IgG antibodies using an ELISA. As shown in Table 7, all of the anti-5T4 antibodies were expressed.

TABLE 7
Expression of Chimeric and Humanized Anti-5T4 Antibodies

| | human IgG in | fold increase in |
|---|---|---|
| | cell culture | expression relative to |
| Antibody | medium (μg/ml) | chimeric H8 |
| Chimeric H8 | 0.5 | — |
| (mVH/mVL) | | |
| Semi-Humanized H8 | 8.4 | 17 |
| (mVH/hVL1) | | |
| Humanized H8 version 1 | 3.6 | 7 |
| (hVH1/hVL1) | | |
| Humanized H8 version 2/1 | 2.0 | 4 |
| (hVH2/hVL1) | | |
| Humanized H8 version 1/2 | 4.3 | 9 |
| (hVH1/hVL2) | | |
| Humanized H8 version 2 | 2.1 | 4 |
| (hVH2/hVL2) | | |

mVH, murine H8 heavy chain variable region
mVL, murine H8 light chain variable region
hVH1, humanized H8 heavy chain variable region version 1
hVL1, humanized H8 light chain variable region version 1
hVH2, humanized H8 heavy chain variable region version 2
hVL2, humanized H8 light chain variable region version 2

To assess the binding specificity and affinity of chimeric and humanized H8 antibodies, BIACORE® analysis was performed using human 5T4 antigen immobilized on a CM5 chip. BIACORE® technology utilizes changes in the refractive index at the surface layer upon binding of the antibody to the 5T4 antigen immobilized on the layer. Binding is detected by surface plasmon resonance (SPR) of laser light refracting from the surface. Analysis of the signal kinetics on rate and off rate allows the discrimination between non-specific and specific interaction. The concentration of antibody used was in the range of 12.5 nM to 200 nM. See Table 8. Chimeric, semi-humanized, and humanized H8 antibodies performed similarly to the murine H8 antibody in the BIACORE® assay.

TABLE 8
Results of BIACORE ® Assay

| Antibody | KD (M) | kd (1/s) | ka (1/Ms) |
|---|---|---|---|
| Murine H8 | 5.8 × 10⁻¹⁰ | 5.9 × 10⁻⁵ | 1.0 × 10⁵ |
| Chimeric H8 | 3.0 × 10⁻¹⁰ | 4.2 × 10⁻⁵ | 1.4 × 10⁵ |
| (mVH/mVL) | | | |
| Humanized H8 version 1 | 8.4 × 10⁻¹⁰ | 7.8 × 10⁻⁵ | 0.9 × 10⁵ |
| (hVH1/hVL1) | | | |
| Humanized H8 version 2 | 1.2 × 10⁻¹⁰ | 1.3 × 10⁻⁵ | 1.1 × 10⁵ |
| (hVH2/hVL2) | | | |
| Humanized H8 version 3 | 1.2 × 10⁻¹⁰ | 1.9 × 10⁻⁵ | 1.5 × 10⁵ |
| (hVH3/hVL2) | | | |

mVH, murine H8 heavy chain variable region
mVL, murine H8 light chain variable region
hVH1, humanized H8 heavy chain variable region version 1
hVL1, humanized H8 light chain variable region version 1
hVH2, humanized H8 heavy chain variable region version 2
hVL2, humanized H8 light chain variable region version 2
hVH3, humanized H8 heavy chain variable region version 3

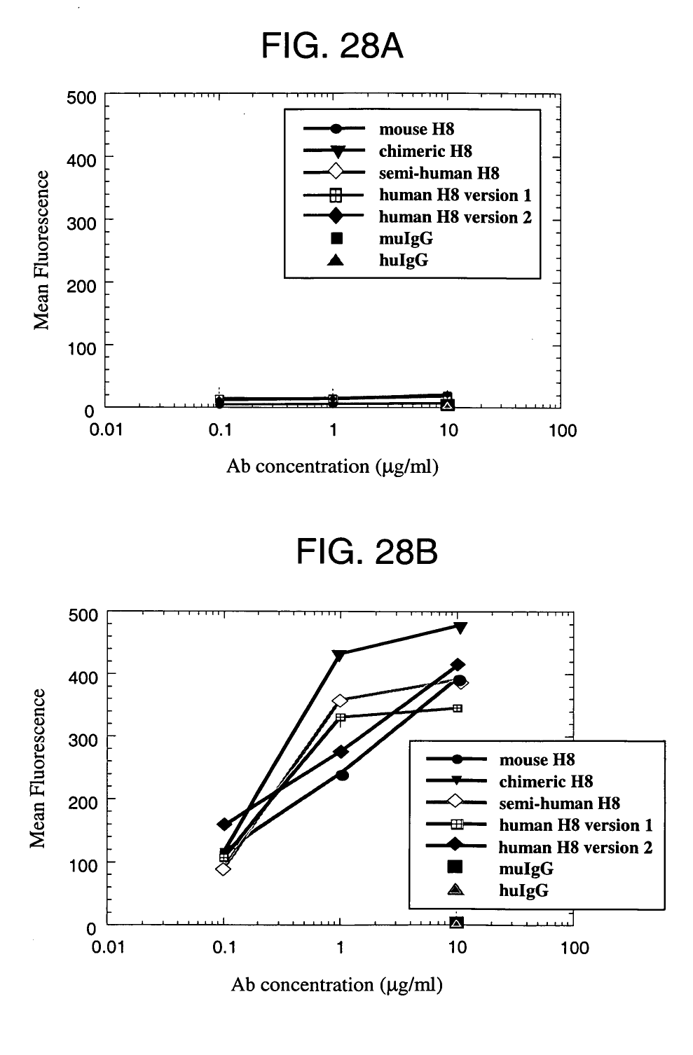

To assess selectivity of binding, FACS analysis was performed to detect 5T4 antigen on MDAMB435/neo cells or on MDAMB435/5T4 cells using murine H8, chimeric versions of H8, and humanized versions of H8 at the indicated concentrations. All antibodies show selective binding to 5T4-expressing cells. See FIGS. 28A-28B.

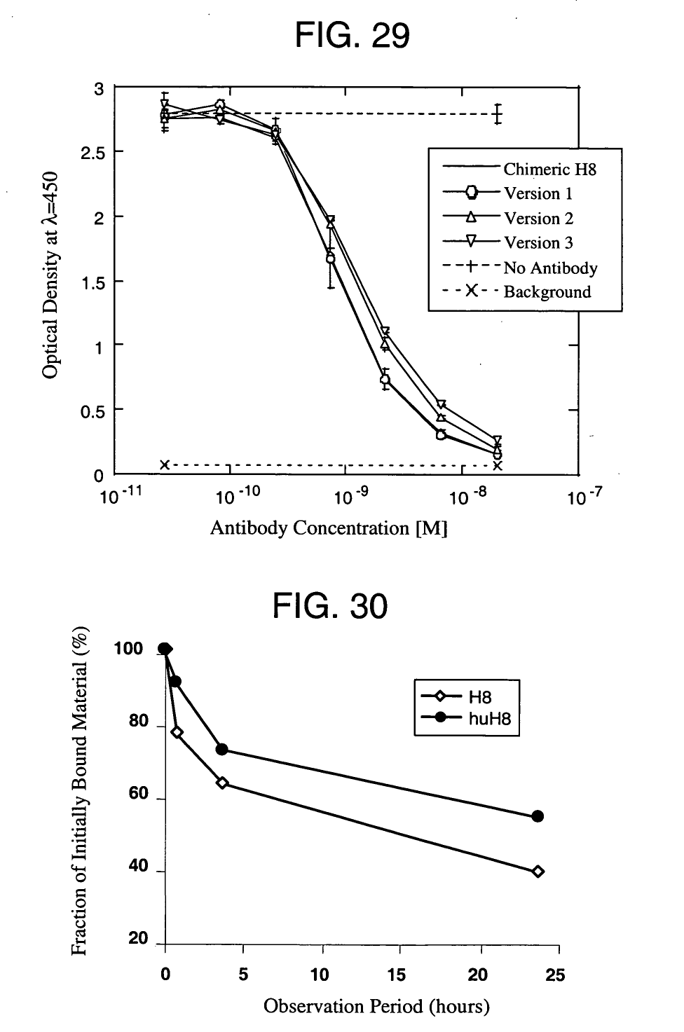

The binding properties of chimeric H8 antibody and humanized H8 versions 1-3 were determined using a competitive binding assay as follows. ELISA plates were coated with human 5T4 antigen. To each well was added 100 μl of 1 μg/ml 5T4 antigen in PBS-CMF pH 7.2. The plates were incubated overnight at 4° C. After coating with antigen, plates were washed in a blocking solution of 0.02% casein in PBS-CMF, pH 7.2 for 2-4 hours at room temperature. Serial dilutions of 250 ng/ml chimeric H8 antibody in assay buffer (0.5% BSA+0.02% TWEEN®-20 in PBS) were prepared, transferred to the coated and blocked ELISA plate, and incubated for 1-2 hours at room temperature. Plates were washed 4 times with 200 μl of 0.03% TWEEN®-20 in PBS. The signal was developed for 10-15 minutes at room temperature following addition of 100 ∥l BIOFX® TMB (Biofx Laboratories, Inc. of Randallstown, Md.) per well. The reaction was stopped by addition of 100 μl per well of 0.18 N H₂SO₄. All incubation and wash steps were performed with gentle agitation. ELISA plates were read at 450 nm. The ED50 of biotinylated antibody binding to antigen was determined by plotting OD₄₅₀ (duplicate data points averaged) as a function of biotinylated antibody concentration. Competition ELISAs were performed by preparing coated and blocked ELISA plates as above, transferring to such plates serial dilutions of test antibody along with biotinylated chimeric H8 antibody at the calculated ED50 concentration. The biotin label was amplified using streptavidin-HRP diluted 1:10,000 in assay buffer, followed by signal development and quantification as above. See FIG. 29.

To assess whether humanized anti-5T4 antibodies are internalized following binding to 5T4 antigen, the amount of antibody detected at the cell surface of MDAMB435/5T4 cells was determined using FACS analysis as described in Example 1. As observed for chimeric H8 antibody and H8 antibody/calicheamicin conjugates, humanized H8 antibodies were internalized by 5T4-expressing cells. See FIG. 30.

### Example 6

### Transient and Stable Expression of Humanized H8 Antibodies

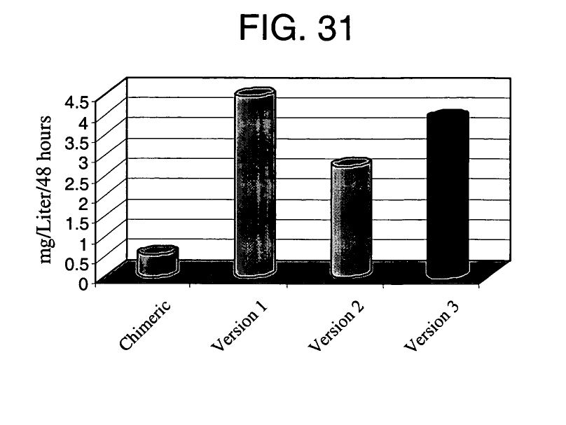

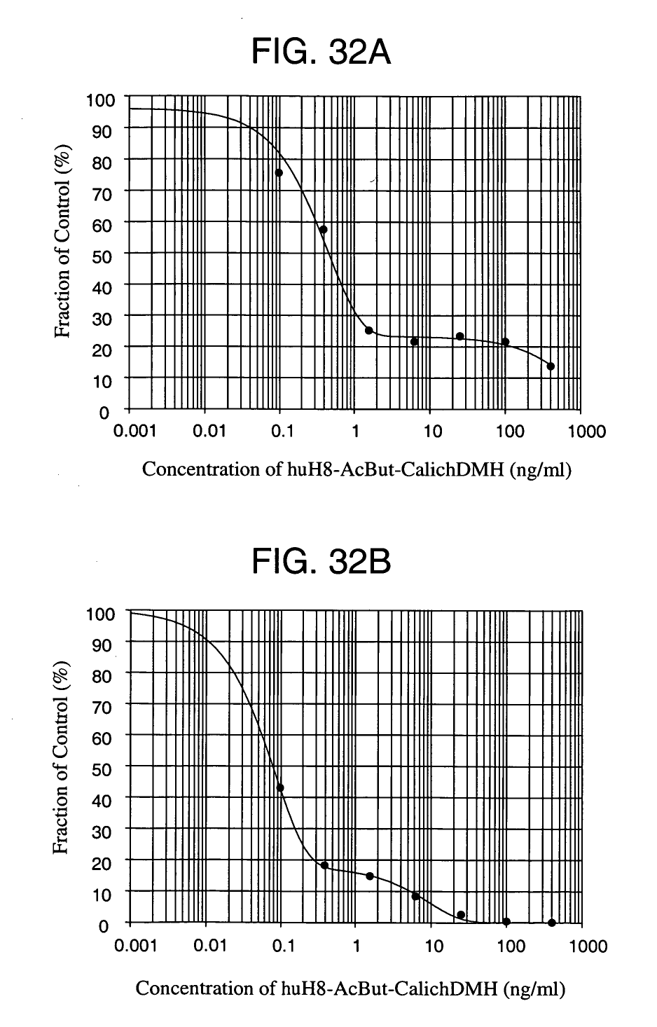

For large scale production of humanized H8 antibodies, stable CHO cell lines that express humanized H8 versions 1-3 were prepared. As an initial step, the level of antibody production was assessed following transient expression of the encoding vectors in COS-1 cells. DNA encoding humanized H8 heavy chains and light chains were subcloned into the bi-cistronic expression vectors pSMED2 (methotrexate resistance) and pSMEN2 (neomycin resistance), respectively. The three humanized H8 antibodies were expressed at similar levels, which were greater than that observed for chimeric H8 antibody. See FIG. 31.

For expression in CHO cells, the human IgG4 mutated constant region was further optimized by removing three introns, which resulted in higher expression and stability of humanized H8 antibodies. CHO cell lines expressing humanized H8 antibodies were prepared by co-transfecting pSMED2_huH8 heavy chain and pSMEN2_huH8 light chain into the pre-adapted CHO Dukx cell line 153.8. The expression level of the lead clone, selected in 50 nM methotrexate, had an average titer of 17 mg/liter/24 hours and an average cellular productivity of 10 μg/10⁶ cells/24 hours. The lead pool, selected in 50 nM methotrexate and G418 (1 mg/ml), had an average titer of 8 mg/liter/24 hours and its average cellular productivity was 6 μg/10⁶ cells/24 hours.

### Example 7

### Preparation and Characterization of Humanized H8-Calicheamicin Conjugates

Humanized H8 antibodies were conjugated to calicheamicin essentially as described in Example 2. Additives deoxycholate and sodium decanoate each produced a conjugate with low levels of unconjugated protein and aggregate. See Table 9.

TABLE 9
Conjugation of Humanized H8 and Calicheamicin at 5 mg Production
Scale

| | End of reaction data | | | |
|---|---|---|---|---|
| | Protein | Loading | | |
| Additive/conc | (mg/mL) | (mcg/mg) | Aggregate | LCF |
| Deoxycholate/10 mM | 5.27 | 78.9 | 7.1 | 1.88 |
| Decanoate/37.5 mM | 4.95 | 84.8 | 7.07 | 0 |

The binding kinetics of murine H8, humanized H8, and humanized H8 calicheamicin conjugate were compared by plasmon resonance, essentially as described in Example 2. The conjugate sample contained 61 μg CalichDMH per mg protein, 1% free antibody, and 1.4% aggregate. The binding properties of humanized H8 version 2-calicheamicin conjugates were comparable to that of murine H8-calicheamicin conjugates (Table 10), indicating that neither humanization of the antibody nor conjugation to calicheamicin affected the binding to 5T4. These results were independently confirmed by determining the binding of the antibodies and conjugates on 5T4 expressing tumor cells using flow cytometry.

TABLE 10
Results of BIACORE ® Assay Using Antibody/Calicheamicin
(CM) Conjugates

| Conjugate | KD (M) | kd (1/s) | ka (1/Ms) |
|---|---|---|---|
| Murine H8/CM | 5.8 × 10⁻¹⁰ | 5.9 × 10⁻⁵ | 1.0 × 10⁵ |
| Humanized H8 version 2/CM | 1.7 × 10⁻¹⁰ | 2.0 × 10⁻⁵ | 1.2 × 10⁵ |
| Humanized H8 version | 2.6 × 10⁻¹⁰ | 3.4 × 10⁻⁵ | 1.3 × 10⁵ |
| 2/AcBut/CalichDMH | | | |

### Example 8

### Anti-Tumor Efficacy of Humanized H8-Calicheamicin Conjugates In Vitro

To assess cytotoxicity of humanized H8-calicheamicin conjugates in vitro, MDAMB435/5T4 cells (human breast carcinoma cells overexpressing human 5T4 antigen) and MDAMB435/neo cells (control cells) were cultured in the presence of antibody-calicheamicin conjugates or free calicheamicin, essentially as described by Boghaert et al. (2004), Clin. Cancer Res., 10: 4538-4549. In Table 11, the cytotoxicity of each agent is reported as ED50 (ng/ml), which is the amount of calicheamicin given as conjugate or as free drug that caused 50% reduction of a cell culture relative to an untreated control. The number of cells in culture was determined using a vital dye (MTS) following 96 hours of drug exposure. The ED50 or the conjugate was consistently lower (3-fold t 6-fold) when added to MDAMB435/5T4 cells than when added to MDAMB435/neo cells.

The cytotoxicity of humanized H8-calicheamicin conjugates was also assessed using MDAMB435/5T4 and MDAMB435/neo cells cultured in a manner suitable for spheroid growth. This model approximates the conditions of a developing tumor and has an inherent greater resistance to cytotoxic drugs. Another advantage of the model is that it allows longer culturing periods. Following 144 hours of culture in the presence of antibody-calicheamicin conjugates or free calicheamicin, the dimensions of each spheroid was determined. As shown in Table 12, the ED50 of huH8-AcBut-CalichDMH was 6-fold lower when added to MDAMB435/5T4 cells than when added to MDAMB435/neo cells.

Using either assay, humanized H8-calicheamicin conjugates were substantially more potent at inducing cytotoxicity and inhibiting spheroid growth when compared to free calicheamicin or to CMA-676, an anti-CD33-calicheamicin conjugate. Selective toxicity of PC14PE6 cells could be demonstrated in a colony forming assay and spheroid assay but not in a vital dye assay. The results demonstrate that the cytotoxicity of the conjugate relates directly to the amount of 5T4 expressed by the cells. In addition the conjugate is more efficacious than free drug (CalichDMH) or control conjugate (CMA-676).

TABLE 11
Results for MTS Assay

| | Cells | | | |
|---|---|---|---|---|
| | | | MDAMB435Q/ | |
| | MDAMB435/5T4 | | neo | |
| | Experiment | | | |
| | A | B | A | B |
| ED50 (ng/ml) CalichDMH | 10 | 4 | 2.3 | 2.2 |
| ED50 (ng/ml) | 0.15 | 0.13 | 0.43 | 0.8 |
| huH8-AcBut-CalichDMH | | | | |
| ED50 (ng/ml) CMA-676 | 40 | >400 | 30 | 70 |
| X-fold potency of huH8-AcBut- | 100 | 31 | 5.3 | 2.8 |
| CalichDMH compared to | | | | |
| CalichDMH | | | | |
| X-fold potency of huH8-AcBut- | 267 | >3,077 | 70 | 80 |
| CalichDMH compared to | | | | |
| CMA-676 | | | | |

CalichDMH, free calicheamicin
huH8-AcBut-CalichDMH, humanized H8 antibody conjugated to calicheamicin using 4-(4′-acetylphenoxy)butanoic acid (AcBut)
CMA-676, anti-CD33 antibody conjugates to calicheamicin
Experiments A & B, experiments performed on separate days

TABLE 12
Results for Spheroid Growth Assay

| | Cells | |
|---|---|---|
| | MDAMB435/5T4 | MDAMB435/neo |
| ED50 (ng/ml) CalichDMH | 1.6 | 2 |
| ED50 (ng/ml) | 0.5 | 0.08 |
| huH8-AcBut-CalichDMH | | |
| ED50 (ng/ml) CMA-676 | 11 | 3.2 |
| X-fold potency of huH8-AcBut- | 3.2 | 25 |
| CalichDMH compared to | | |
| CalichDMH | | |
| X-fold potency of huH8-AcBut- | 22 | 40 |
| CalichDMH compared to | | |
| CMA-676 | | |

CalichDMH, free calicheamicin
huH8-AcBut-CalichDMH, humanized H8 antibody conjugated to calicheamicin using 4-(4′-acetylphenoxy)butanoic acid (AcBut)
CMA-676, anti-CD33 antibody conjugates to calicheamicin

### Example 9

### Anti-Tumor Efficacy of Humanized H8-Calicheamicin Conjugates Using Subcutaneous Xenografts

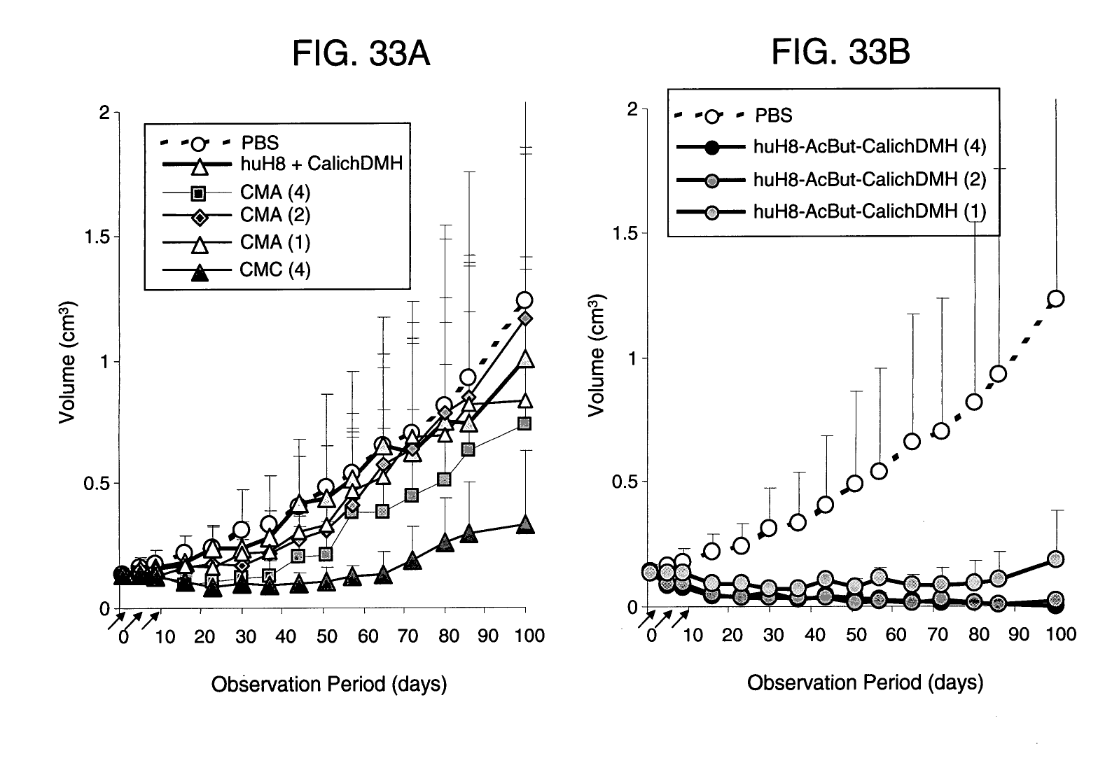

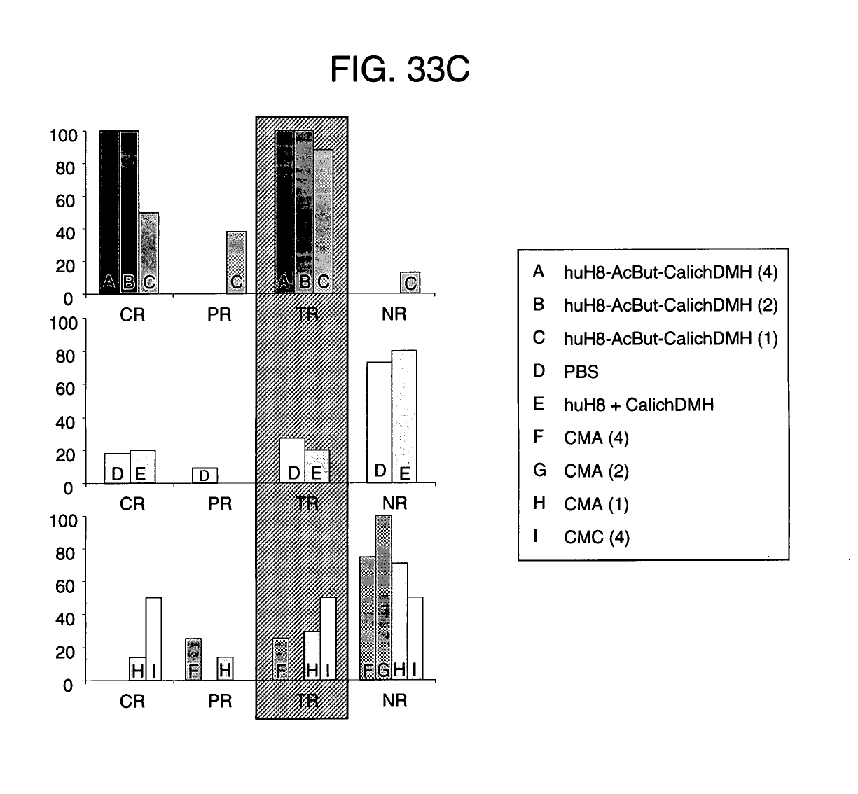

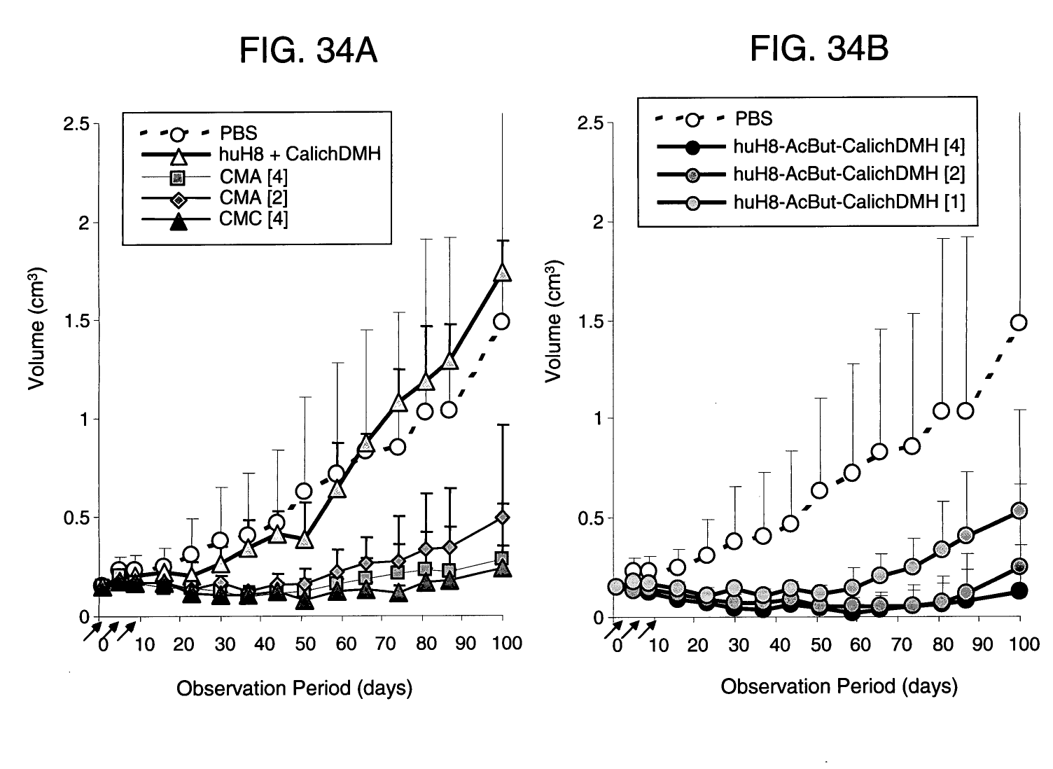

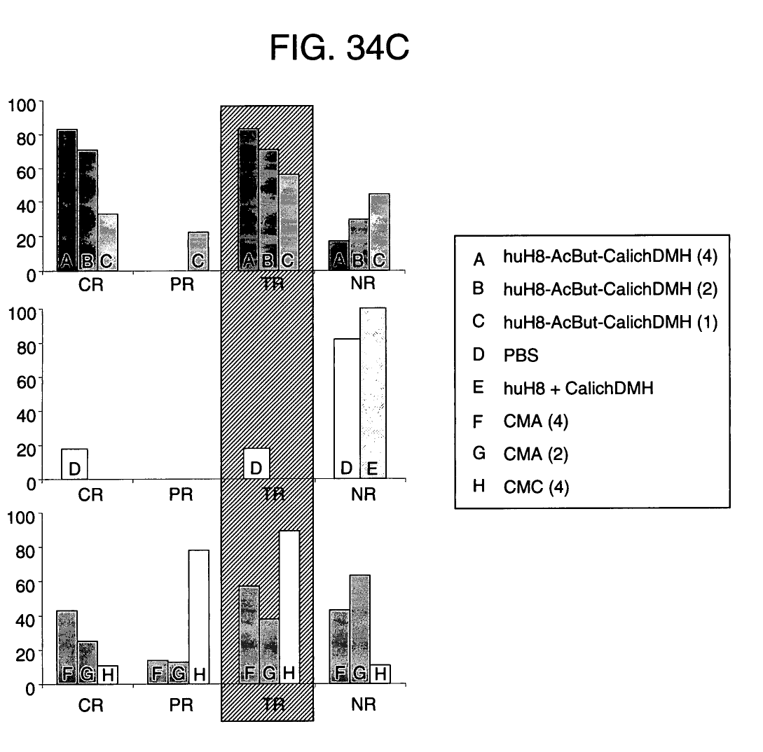

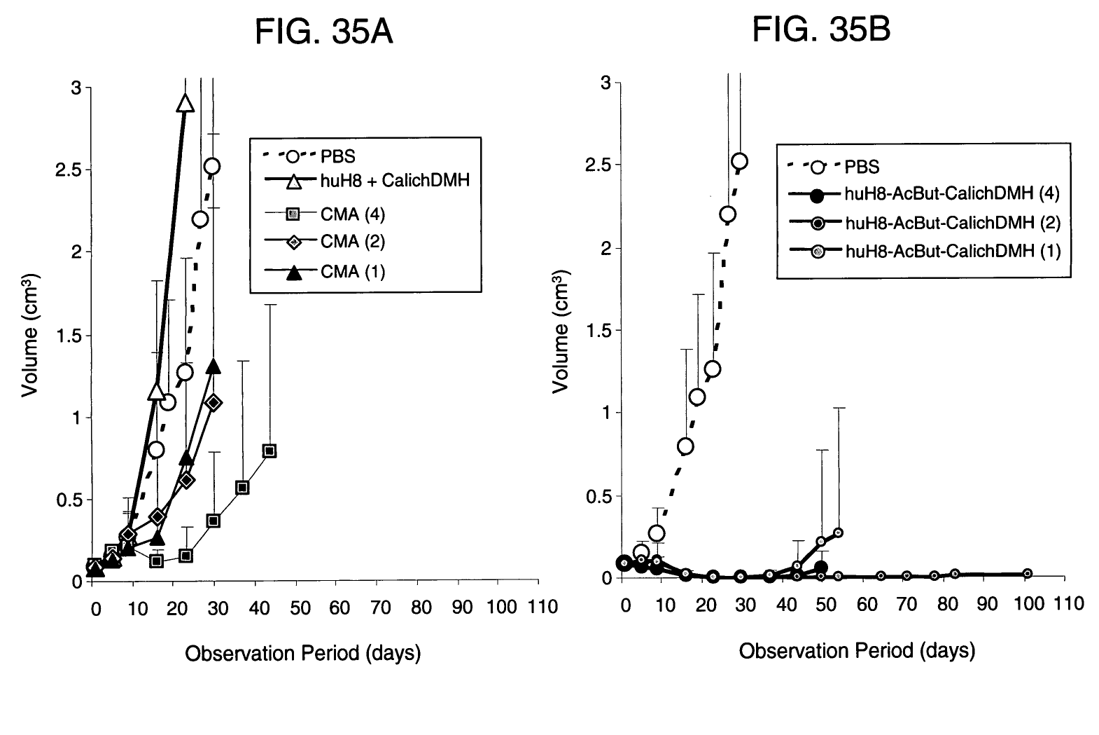

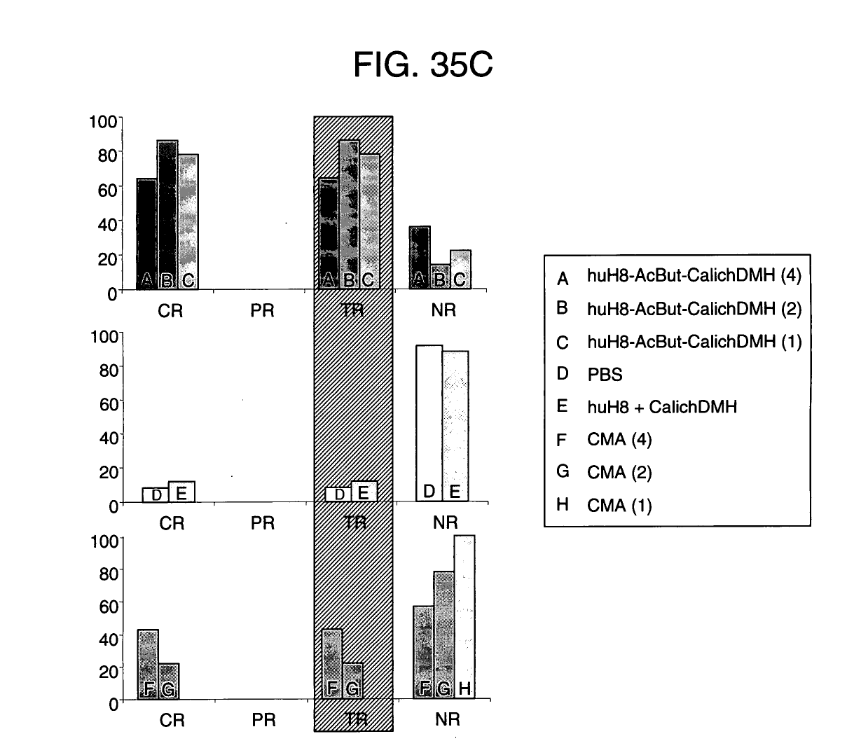

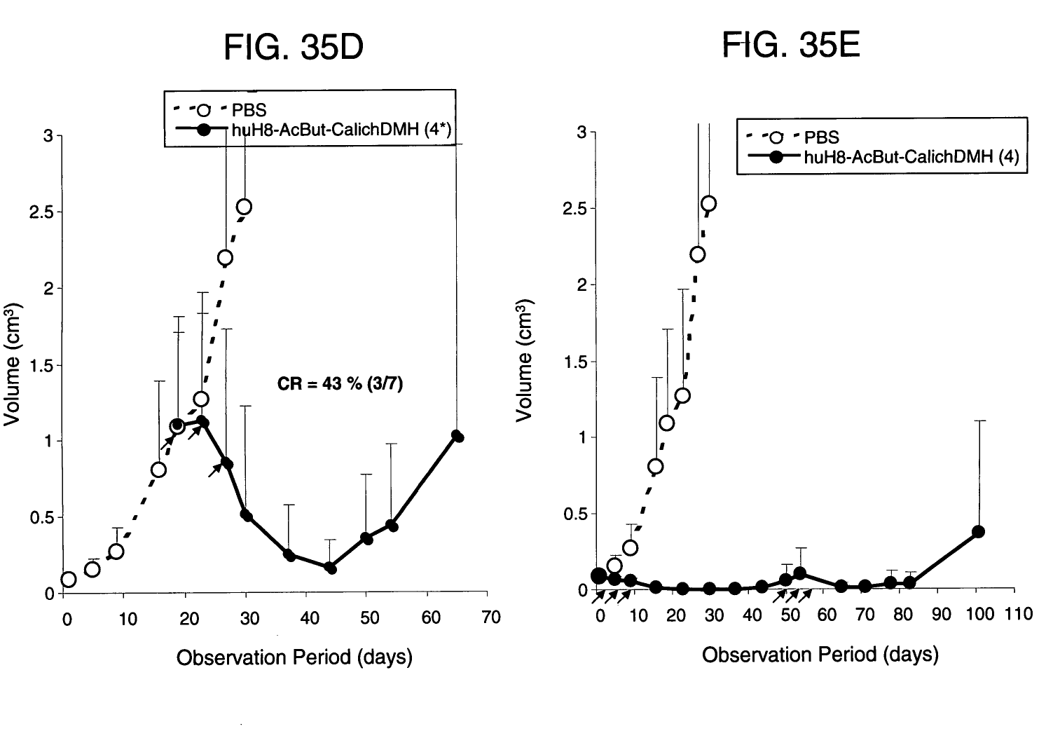

To assess the cytotoxicity of humanized H8-calicheamicin conjugates in vivo, tumors were prepared in nude mice by subcutaneous injection of N87 cells (human gastric carcinoma cells), MDAMB435/5T4 cells (human breast carcinoma cells overexpressing human 5T4 antigen), or PC14PE6 cells (human non-small cell lung cancer cells). Humanized H8-calicheacmicin conjugates and control agents were administered by intraperitoneal injection to tumor-bearing mice in a total of 3 doses given at 4-day intervals, i.e., on days 1, 5, and 9 following selection of tumors having achieved a size of approximately 0.08 cm³ (FIGS. 33A-33C, 34A-34C, 35A-35C, and 35E) or on days 19, 23, and 27 following selection of tumors having achieved a size of 1.08 cm³ (FIG. 35D). In one study, animals bearing relapsed tumors were treated (FIG. 35E). A total of 11 animals were treated with humanized H8-calicheamicin conjugates, and 13 animals were treated with the indicated control substances.

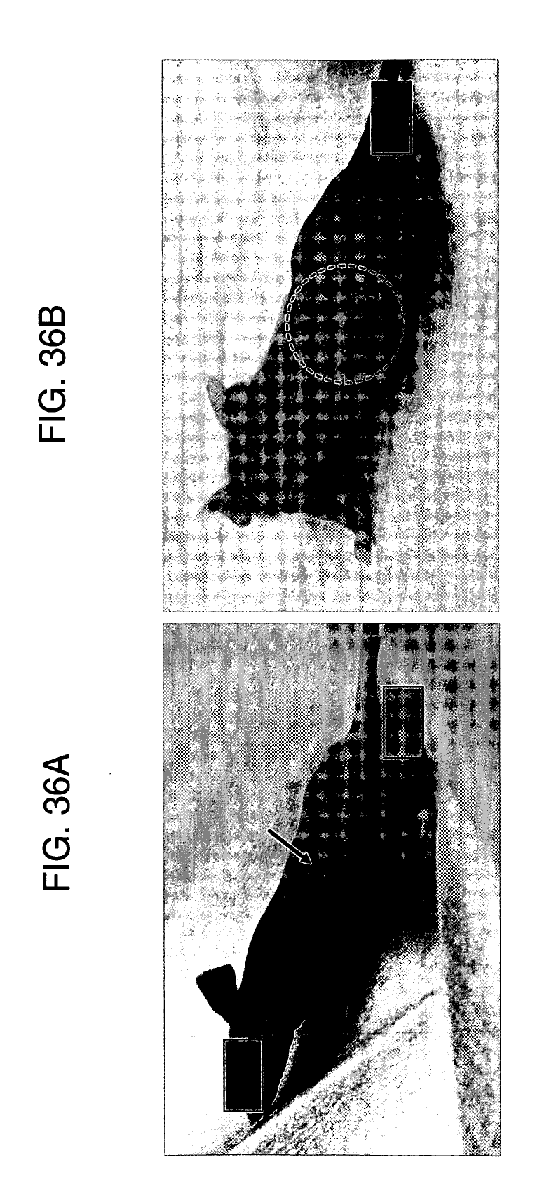

Response rates to therapy were determined 99 days after the first dose. Complete response rate (CR) is the percentage of surviving mice with a tumor size smaller or equal to the average initial tumor volume of the group. Partial response rate (PR) is the percentage of surviving mice with a tumor size smaller or equal to twice the average initial tumor volume of the group. Total response (TR) is the sum of CR and PR. No response (NR) is calculated as (100-TR). See FIGS. 33C, 34C, and 35C. Humanized H8-calicheamicin conjugates inhibited growth of all tumor types. See FIGS. 33A-33B, 34A-34B, 35A-35D, and 36A-36B. The amount of huH8-AcBut-CalichDMH needed to inhibit PC14PE6 cells, i.e, the minimal effective dose, is at least 16-fold lower than the maximal non-lethal dose.

1. A chimeric or humanized anti-5T4 antibody comprising at least one light chain or at least one heavy chain, or a chimeric or humanized fragment thereof, wherein the antibody or antibody fragment
(a) specifically binds to human 5T4 antigen with a binding affinity of at least about 1×10⁻⁷ M to about 1×10⁻¹² M;
(b) specifically binds to human 5T4 antigen with a binding affinity greater than 1×10⁻¹¹ M;
(c) specifically binds to human 5T4 antigen with a binding affinity greater than 5×10⁻¹¹ M;
(d) specifically binds to human 5T4 antigen with a binding affinity greater than a binding affinity of murine H8 anti-5T4 antibody binding to human 5T4 antigen;
(e) specifically targets 5T4-expressing cells in vivo;
(f) competes for binding to human 5T4 antigen with an antibody of any one of (a)-(e);
(g) specifically binds to an epitope bound by any one of (a)-(e); or
(h) comprises an antigen binding domain of any one of (a)-(e).

2. The chimeric or humanized anti-5T4 antibody or antibody fragment of claim 1 comprising constant regions derived from human constant regions.

3. The chimeric or humanized anti-5T4 antibody or antibody fragment of claim 2, wherein the human light chain constant region is derived from human kappa light chain constant region.

4. The chimeric or humanized anti-5T4 antibody or antibody fragment of claim 2, wherein the human heavy chain constant region is derived from a human IgG1 or human IgG4 heavy chain constant region.

5. The chimeric or humanized anti-5T4 antibody or antibody fragment of claim 4, wherein the human IgG1 heavy chain constant region comprises an amino acid sequence of any one of SEQ ID NOs:25 or 85-89.

6. The chimeric or humanized anti-5T4 antibody or antibody fragment of claim 4, wherein the human IgG4 heavy chain constant region comprises proline at position 241.

7. The chimeric anti-5T4 antibody or antibody fragment of claim 1, wherein the light chain variable region sequence comprises amino acids 1-107 of SEQ ID NO:1.

8. The chimeric anti-5T4 antibody or antibody fragment of claim 1, wherein the heavy chain variable region sequence comprises amino acids 1-120 of SEQ ID NO:2.

9. The chimeric anti-5T4 antibody or antibody fragment of claim 1, wherein the light chain comprises a variable region comprising an amino acid sequence of residues 1-107 of SEQ ID NO:1, and wherein the heavy chain comprises a variable region comprising an amino acid sequence of residues 1-120 of SEQ ID NO:2.

10. The chimeric anti-5T4 antibody or antibody fragment of claim 1, wherein
(a) the light chain comprises an amino acid sequence of SEQ ID NO:1, and the heavy chain comprises an amino acid sequence of SEQ ID NO:2; or
(b) the light chain comprises an amino acid sequence of SEQ ID NO:3, and the heavy chain comprises an amino acid sequence of SEQ ID NO:4.

11. The humanized anti-5T4 antibody or antibody fragment of claim 1, wherein the variable region of the at least one light chain or at least one heavy chain comprises:
(a) framework regions comprising residues of a human antibody framework region; and
(b) one or more CDRs of the light chain variable region of SEQ ID NO:17 or one or more CDRs of the heavy chain variable region of SEQ ID NO:18.

12. The humanized anti-5T4 antibody or antibody fragment of claim 11, wherein the framework regions comprise:
(a) a human antibody light chain framework region of a DPK24 subgroup IV germ line clone, a VκIII subgroup, or a VκI subgroup germ line clone;
(b) a human antibody heavy chain framework region selected from the group consisting of DP-75, DP-8(VH1-2), DP-25, VI-2b and VI-3 (VH1-03), DP-15 and V1-8 (VH1-08), DP-14 and V1-18 (VH1-18), DP-5 and V1-24P (VH1-24), DP-4 (VH1-45), DP-7 (VH1-46), DP-10, DA-6 and YAC-7 (VH1-69), DP-88 (VH1-e), DP-3 and DA-8 (VH1-f);
(c) a consensus sequence of a heavy chain framework region of (b); or
(d) a framework region that is at least 95% identical to a framework region of (a)-(c).

13. The humanized anti-5T4 antibody or antibody fragment of claim 11 comprising at least two CDRs of SEQ ID NOs:17 or 18.

14. The humanized anti-5T4 antibody or antibody fragment of claim 13, wherein the light chain comprises a variable region comprising at least two of three CDRs of SEQ ID NO:17.

15. The humanized anti-5T4 antibody or antibody fragment of claim 14, wherein the light chain comprises a variable region comprising three CDRs of SEQ ID NO:17.

16. The humanized anti-5T4 antibody or antibody fragment of claim 11, wherein the heavy chain comprises a variable region comprising at least two of three CDRs of SEQ ID NO:18.

17. The humanized anti-5T4 antibody or antibody fragment of claim 16, wherein the heavy chain comprises a variable region comprising three CDRs of SEQ ID NO:18.

18. The humanized anti-5T4 antibody or antibody fragment of claim 11, wherein the light chain comprises the CDRs of SEQ ID NOs:17-18.

19. The humanized anti-5T4 antibody or antibody fragment of claim 1, wherein the light chain variable region sequence comprises:
(a) an amino acid sequence of SEQ ID NO:17 or 23;
(b) an amino acid sequence that is at least 78% identical to SEQ ID NO:17; or
(c) an amino acid sequence that is at least 81% identical to SEQ ID NO:23.

20. The humanized anti-5T4 antibody or antibody fragment of claim 1, wherein the light chain variable region sequence is encoded by a nucleic acid comprising:
(a) a nucleotide sequence of SEQ ID NO:22 or 81;
(b) a nucleotide sequence that is at least 90% identical to the nucleic acid of SEQ ID NO:22;
(c) a nucleotide sequence that is at least 91% identical to the nucleic acid of SEQ ID NO:81; or
(d) a nucleic acid that specifically hybridizes to the complement of SEQ ID NO:22 or SEQ ID NO:81 under stringent hybridization conditions.

21. The humanized anti-5T4 antibody or antibody fragment of claim 1, wherein the heavy chain variable region sequence comprises:
(a) an amino acid sequence set forth as any one of SEQ ID NOs:18, 19, and 21;
(b) an amino acid sequence that is at least 83% identical to SEQ ID NO:18;
(c) an amino acid sequence that is at least 81% identical to SEQ ID NO:19; or
(d) an amino acid sequence that is at least 86% identical to SEQ ID NO:21.

22. The humanized anti-5T4 antibody or antibody fragment of claim 1, wherein the heavy chain variable region sequence is encoded by a nucleic acid comprising:
(a) a nucleotide sequence of SEQ ID NO:20, 82, or 83;
(b) a nucleotide sequence that is at least 91% identical to the nucleic acid of SEQ ID NO:20 or SEQ ID NO:83;
(c) a nucleotide sequence that is at least 94% identical to the nucleic acid of SEQ ID NO:82; or
(d) a nucleic acid that specifically hybridizes to the complement of any one of SEQ ID NOs:20, 82, and 83 under stringent hybridization conditions.

23. A humanized anti-5T4 antibody or antibody fragment comprising:
(a) a light chain variable region comprising an amino acid sequence of residues 1-107 of SEQ ID NO:5, and a heavy chain variable region comprising an amino acid sequence of residues 1-120 of SEQ ID NO:6; or
(b) a light chain amino acid sequence of SEQ ID NO:5, and a heavy chain amino acid sequence of SEQ ID NO:6.

24. A humanized anti-5T4 antibody or antibody fragment comprising:
(a) a light chain variable region comprising an amino acid sequence of residues 1-107 of SEQ ID NO:7, and a heavy chain variable region comprising an amino acid sequence of residues 1-120 of SEQ ID NO:8; or
(b) a light chain amino acid sequence of SEQ ID NO:7, and a heavy chain amino acid sequence of SEQ ID NO:8.

25. A humanized anti-5T4 antibody or antibody fragment comprising:
(a) a light chain variable region comprising an amino acid sequence of residues 1-107 of SEQ ID NO:9, and a heavy chain variable region comprising an amino acid sequence of residues 1-120 of SEQ ID NO:10; or
(b) a light chain amino acid sequence of SEQ ID NO:9, and a heavy chain amino acid sequence of SEQ ID NO:10.

26. A humanized anti-5T4 antibody or antibody fragment comprising:
(a) a light chain variable region comprising an amino acid sequence of residues 1-107 of SEQ ID NO:11, and a heavy chain variable region comprising an amino acid sequence of residues 1-120 of SEQ ID NO:12; or
(b) a light chain amino acid sequence of SEQ ID NO:11, and a heavy chain amino acid sequence of SEQ ID NO:12.

27. A humanized anti-5T4 antibody or antibody fragment comprising:
(a) a light chain variable region comprising an amino acid sequence of residues 1-107 of SEQ ID NO:11, and a heavy chain variable region comprising an amino acid sequence of SEQ ID NO:19; or
(b) a light chain amino acid sequence of SEQ ID NO:11, and a heavy chain amino acid sequence of SEQ ID NO:84.

28. A humanized anti-5T4 antibody or antibody fragment comprising:
(a) a light chain variable region comprising an amino acid sequence of residues 1-107 of SEQ ID NO:11, and a heavy chain variable region comprising an amino acid sequence of residues 1-120 of SEQ ID NO:8; or
(b) a light chain amino acid sequence of SEQ ID NO:11, and a heavy chain amino acid sequence of SEQ ID NO:8.

29. An antibody/drug conjugate for drug delivery comprising:
(a) a chimeric or humanized anti-5T4 antibody or antibody fragment; and
(b) a drug, which is directly or indirectly bound to the antibody or antibody fragment.

30. The antibody/drug conjugate of claim 29, wherein the chimeric or humanized anti-5T4 antibody or antibody fragment:
(a) specifically binds to human 5T4 antigen with a binding affinity of at least about 1×10⁻⁷ M to about 1×10⁻¹² M;
(b) specifically binds to human 5T4 antigen with a binding affinity greater than 1×10⁻¹¹ M;
(c) specifically binds to human 5T4 antigen with a binding affinity greater than 5×10⁻¹¹ M;
(d) specifically binds to human 5T4 antigen with a binding affinity greater than a binding affinity of murine H8 anti-5T4 antibody binding to human 5T4 antigen;
(e) specifically targets 5T4-expressing cells in vivo;
(f) competes for binding to human 5T4 antigen with an antibody of any one of (a)-(e);
(g) specifically binds to an epitope bound by any one of (a)-(e); or
(h) comprises an antigen binding domain of any one of (a)-(e)

31. The antibody/drug conjugate of claim 29, wherein the chimeric or humanized anti-5T4 antibody or antibody fragment comprises:
(a) a light chain variable region comprising an amino acid sequence of residues 1-107 of SEQ ID NO:5, and a heavy chain variable region comprising an amino acid sequence of residues 1-120 of SEQ ID NO:6; or
(b) a light chain amino acid sequence of SEQ ID NO:5, and a heavy chain amino acid sequence of SEQ ID NO:6.

32. The antibody/drug conjugate of claim 29, wherein the chimeric or humanized anti-5T4 antibody or antibody fragment comprises:
(a) a light chain variable region comprising an amino acid sequence of residues 1-107 of SEQ ID NO:7, and a heavy chain variable region comprising an amino acid sequence of residues 1-120 of SEQ ID NO:8; or
(b) a light chain amino acid sequence of SEQ ID NO:7, and a heavy chain amino acid sequence of SEQ ID NO:8.

33. The antibody/drug conjugate of claim 29, wherein the chimeric or humanized anti-5T4 antibody or antibody fragment comprises:
(a) a light chain variable region comprising an amino acid sequence of residues 1-107 of SEQ ID NO:9, and a heavy chain variable region comprising an amino acid sequence of residues 1-120 of SEQ ID NO:10; or
(b) a light chain amino acid sequence of SEQ ID NO:9, and a heavy chain amino acid sequence of SEQ ID NO:10.

34. The antibody/drug conjugate of claim 29, wherein the chimeric or humanized anti-5T4 antibody or antibody fragment comprises:
(a) a light chain variable region comprising an amino acid sequence of residues 1-107 of SEQ ID NO:11, and a heavy chain variable region comprising an amino acid sequence of residues 1-120 of SEQ ID NO:12; or
(b) a light chain amino acid sequence of SEQ ID NO:11, and a heavy chain amino acid sequence of SEQ ID NO:12.

35. The antibody/drug conjugate of claim 29, wherein the chimeric or humanized anti-5T4 antibody or antibody fragment comprises:
(a) a light chain variable region comprising an amino acid sequence of residues 1-107 of SEQ ID NO:11, and a heavy chain variable region comprising an amino acid sequence of SEQ ID NO:19; or
(b) a light chain amino acid sequence of SEQ ID NO:11, and a heavy chain amino acid sequence of SEQ ID NO:84.

36. The antibody/drug conjugate of claim 29, wherein the chimeric or humanized anti-5T4 antibody or antibody fragment comprises:
(a) a light chain variable region comprising an amino acid sequence of residues 1-107 of SEQ ID NO:11, and a heavy chain variable region comprising an amino acid sequence of residues 1-120 of SEQ ID NO:8; or
(b) a light chain amino acid sequence of SEQ ID NO:11, and a heavy chain amino acid sequence of SEQ ID NO:8.

37. The antibody/drug conjugate of claim 29, wherein the drug is a therapeutic agent selected from the group consisting of a cytotoxin, a radioisotope, an immunomodulatory agent, an anti-angiogenic agent, an anti-proliferative agent, a pro-apoptotic agent, a chemotherapeutic agent, and a therapeutic nucleic acid.

38. The antibody/drug conjugate of claim 37, wherein the therapeutic agent is a cytotoxin.

39. The antibody/drug conjugate of claim 38, wherein the cytotoxin is an antibiotic, an inhibitor of tubulin polymerization, an alkylating agent, a protein synthesis inhibitor, a protein kinase inhibitor, a phosphatase inhibitor, a topoisomerase inhibitor, or an enzyme.

40. The antibody/drug conjugate of claim 39, wherein the cytotoxin is an antibiotic.

41. The antibody/drug conjugate of claim 40, wherein the antibiotic is calicheamicin.

42. The antibody/drug conjugate of claim 41, wherein the calicheamicin is an N-acetyl derivative or disulfide analog of calicheamicin.

43. The antibody/drug conjugate of claim 42, wherein the calicheamicin is N-acetyl-γ-calicheamicin.

44. The antibody/drug conjugate of claim 29, wherein the drug is bound to the antibody via a linker.

45. The antibody/drug conjugate of claim 44, wherein the linker is selected from the group consisting of 4-(4′acetylphenoxy)butanoic acid (AcBut), 3-acetylphenyl acidic acid (AcPac), 4-mercapto-4-methyl-pentanoic acid (Amide), and derivatives thereof.

46. A method for delivering a drug to 5T4-expressing cells comprising contacting the cells with an antibody/drug conjugate comprising (i) a chimeric or humanized anti-5T4 antibody or antibody fragment, and (ii) a drug which is bound to the humanized anti-5T4 antibody or antibody fragment directly or indirectly.

47. The method of claim 46, wherein the drug is internalized in a target cell.

48. A method for treating a subject having a 5T4-positive cancer, said method comprising administering to the subject a therapeutically effective amount of an anti-5T4 antibody/drug conjugate comprising (i) a chimeric or humanized anti-5T4 antibody or antibody fragment, and (ii) a therapeutic agent which is bound to the chimeric or humanized anti-5T4 antibody or antibody fragment directly or indirectly.

49. The method of claim 48, wherein the anti-5T4 antibody/drug conjugate is a anti-5T4 antibody/calicheamicin conjugate, and further comprising administering a second therapeutic agent, wherein the anti-5T4/calicheamicin conjugate and the second therapeutic agent are administered concurrently or consecutively in either order.


SEQUENCE LISTING (89 sequences)

SEQ ID NO: 1 - protein, 214 aa, Artificial
  artificial sequence is derived from mouse and human antibody sequences
SIVMTQTPTFLLVSAGDRVTITCKASQSVSNDVAWYQQKPGQSPTLLISYTSSRYAGVPDRFIGSGYGTDFTFTISTLQAEDLAVYFCQQDYNSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial
  artificial sequence is derived from mouse and human antibody sequences
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial; identical to SEQ ID NO: 1
  artificial sequence is derived from mouse and human antibody sequences

SEQ ID NO: 4 - protein, 447 aa, Artificial
  artificial sequence is derived from mouse and human antibody sequences
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 214 aa, Artificial
  artificial sequence is derived from mouse and human antibody sequences
DIVMTQSPDSLAVSLGERATINCKASQSVSNDVAWYQQKPGQSPKLLISYTSSRYAGVPDRFSGSGSGTDFTLTISSLQAEDVAVYFCQQDYNSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 447 aa, Artificial; identical to SEQ ID NO: 4
  artificial sequence is derived from mouse and human antibody sequences

SEQ ID NO: 7 - protein, 214 aa, Artificial; identical to SEQ ID NO: 5
  artificial sequence is derived from mouse and human antibody sequences

SEQ ID NO: 8 - protein, 447 aa, Artificial
  artificial sequence is derived from mouse and human antibody sequences
QVQLVQSGAEVKKPGASVKVSCKASGYSFTGYYMHWVKQSPGQGLEWIGRINPNNGVTLYNQKFKDRVTMTRDTSISTAYMELSRLRSDDTAVYYCARSTMITNYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 214 aa, Artificial; identical to SEQ ID NO: 5
  artificial sequence is derived from mouse and human antibody sequences

SEQ ID NO: 10 - protein, 447 aa, Artificial
  artificial sequence is derived from mouse and human antibody sequences
QVQLVQSGAEVKKPGASVKVSCKASGYSFTGYYMHWVRQAPGQGLEWMGRINPNNGVTLYNQKFKDRVTMTRDTSISTAYMELSRLRSDDTAVYYCARSTMITNYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial
  artificial sequence is derived from mouse and human antibody sequences
DIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 447 aa, Artificial; identical to SEQ ID NO: 10
  artificial sequence is derived from mouse and human antibody sequences

SEQ ID NO: 13 - DNA, 417 bp, Mus musculus
atggaatggagctgggtctttctcttcctcctgtcagtaactacaggtgtccactctgaggtccagctgcagcagtctggacctgacctggtgaagcctggggcttcagtgaagatatcctgcaaggcttctggttactcattcactggctactacatgcactgggtgaagcagagccatggaaagagccttgagtggattggacgtattaatcctaacaatggtgttactctctacaaccagaaattcaaggacaaggccatattaactgtagacaagtcatccaccacagcctacatggagctccgcagcctgacatctgaggactctgcggtctattactgtgcaagatctactatgattacgaactatgttatggactactggggtcaagtaacctcagtcaccgtctcctca

SEQ ID NO: 14 - protein, 139 aa, Mus musculus
  MISC_FEATURE (1)..(19): leader sequence
MEWSWVFLFLLSVTTGVHSEVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSS

SEQ ID NO: 15 - DNA, 381 bp, Mus musculus
atgaagtcacagacccaggtcttcgtatttctactgctctgtgtgtctggtgcgcatgggagtattgtgatgacccagactcccacattcctgcttgtttcagcaggagacagggttaccataacctgcaaggccagtcagagtgtgagtaatgatgtagcttggtaccaacagaagccagggcagtctcctacactgctcatatcctatacatccagtcgctacgctggagtccctgatcgcttcattggcagtggatatgggacggatttcactttcaccatcagcactttgcaggctgaagacctggcagtttatttctgtcagcaagattataattctcctccgacgttcggtggaggcaccaagctggaaatcaaa

SEQ ID NO: 16 - protein, 127 aa, Mus musculus
  MISC_FEATURE (1)..(20): leader sequence
MKSQTQVFVFLLLCVSGAHGSIVMTQTPTFLLVSAGDRVTITCKASQSVSNDVAWYQQKPGQSPTLLISYTSSRYAGVPDRFIGSGYGTDFTFTISTLQAEDLAVYFCQQDYNSPPTFGGGTKLEIK

SEQ ID NO: 17 - protein, 107 aa, Artificial
  artificial sequence is derived from mouse and human antibody sequence
DIVMTQSPDSLAVSLGERATINCKASQSVSNDVAWYQQKPGQSPKLLISYTSSRYAGVPDRFSGSGSGTDFTLTISSLQAEDVAVYFCQQDYNSPPTFGGGTKLEIK

SEQ ID NO: 18 - protein, 120 aa, Artificial
  artificial sequence is derived from mouse and human antibody sequences
QVQLVQSGAEVKKPGASVKVSCKASGYSFTGYYMHWVKQSPGQGLEWIGRINPNNGVTLYNQKFKDRVTMTRDTSISTAYMELSRLRSDDTAVYYCARSTMITNYVMDYWGQGTLVTVSS

SEQ ID NO: 19 - protein, 120 aa, Artificial
  artificial sequence is derived from mouse and human antibody sequences
QVQLVQSGAEVKKPGASVKVSCKASGYSFTGYYMHWVRQAPGQGLEWMGRINPNNGVTLYNQKFKDRVTITRDTSTSTAYMELSSLRSEDTAVYYCARSTMITNYVMDYWGQGTLVTVSS

SEQ ID NO: 20 - DNA, 360 bp, Artificial
  artificial sequence is derived from mouse and human antibody sequences
caggtccagctggtgcagtctggagccgaggtgaagaagcctggggcttcagtgaaggtgtcctgcaaggcttctggttactcattcactggctactacatgcactgggtgcgccaggcccccggacagggccttgagtggatgggacgtattaatcctaacaatggtgttactctctacaaccagaaattcaaggaccgcgtgaccatgactcgcgacacctccatctccacagcctacatggagctctcccgcctgcgctctgacgacaccgccgtctattactgtgcacgctccactatgattaccaactatgttatggactactggggtcaaggcaccctggtcaccgtctcctca

SEQ ID NO: 21 - protein, 120 aa, Artificial
  artificial sequence is derived from mouse and human antibody sequences
QVQLVQSGAEVKKPGASVKVSCKASGYSFTGYYMHWVRQAPGQGLEWMGRINPNNGVTLYNQKFKDRVTMTRDTSISTAYMELSRLRSDDTAVYYCARSTMITNYVMDYWGQGTLVTVSS

SEQ ID NO: 22 - DNA, 321 bp, Artificial
  artificial sequence is derived from mouse and human antibody sequences
gatattgtgatgacccagtcccccgactccctggccgtttcactgggagagagggccaccataaactgcaaggccagtcagagtgtgagtaatgatgtggcttggtaccaacagaagccagggcagccccctaagctgctcatatactatacatccagtcgctacgctggagtccctgatcgcttctccggcagtggatccgggaccgatttcactctgaccatcagctccttgcaggctgaagacgtggcagtttattactgtcagcaagattataattctcctcccaccttcggtggaggcaccaagctggaaatcaaa

SEQ ID NO: 23 - protein, 107 aa, Artificial
  artificial sequence is derived from mouse and human antibody sequences
DIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIK

SEQ ID NO: 24 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 114 bp, Artificial
  oligonucleotide
cgcgcactccgatattgtgatgacccagtcccccgactccctggccgtttcactgggagagagggccaccataaactgcaaggccagtcagagtgtgagtaatgatgtggcttg

SEQ ID NO: 28 - DNA, 117 bp, Artificial
  oligonucleotide
gtaccaacagaagccagggcagtcccctaagctgctcatatcctatacatccagtcgctacgctggagtccctgatcgcttctccggcagtggatccgggaccgatttcactctgac

SEQ ID NO: 29 - DNA, 120 bp, Artificial
  oligonucleotide
catcagctccttgcaggctgaagacgtggcagtttatttctgtcagcaagattataattctcctcccaccttcggtggaggcaccaagctggaaatcaaacgtgagtagaataacttaat

SEQ ID NO: 30 - DNA, 105 bp, Artificial
  oligonucleotide
taagttattctactcacgtttgatttccagcttggtgcctccaccgaaggtgggaggagaattataatcttgctgacagaaataaactgccacgtcttcagcctg

SEQ ID NO: 31 - DNA, 116 bp, Artificial
  oligonucleotide
caaggagctgatggtcagagtgaaatcggtcccggatccactgccggagaagcgatcagggactccagcgtagcgactggatgtataggatatgagcagcttaggggactgccctg

SEQ ID NO: 32 - DNA, 124 bp, Artificial
  oligonucleotide
gcttctgttggtaccaagccacatcattactcacactctgactggccttgcagtttatggtggccctctctcccagtgaaacggccagggagtcgggggactgggtcatcacaatatcggagtg

SEQ ID NO: 33 - DNA, 27 bp, Artificial
  PCR primer
cagccccctaagctgctcatatactat

SEQ ID NO: 34 - DNA, 27 bp, Artificial
  PCR primer
atagtatatgagcagcttagggggctg

SEQ ID NO: 35 - DNA, 25 bp, Artificial
  PCR primer
gtggcagtttattactgtcagcaag

SEQ ID NO: 36 - DNA, 25 bp, Artificial
  PCR primer
cttgctgacagtaataaactgccac

SEQ ID NO: 37 - DNA, 86 bp, Artificial
  oligonucleotide
cgcgcactcccaggtccagctggtgcagtctggagccgaggtgaagaagcctggggcttcagtgaaggtgtcctgcaaggcttctg

SEQ ID NO: 38 - DNA, 90 bp, Artificial
  oligonucleotide
gttactcattcactggctactacatgcactgggtgaagcagagccccggacagggccttgagtggattggacgtattaatcctaacaatg

SEQ ID NO: 39 - DNA, 101 bp, Artificial
  oligonucleotide
gtgttactctctacaaccagaaattcaaggaccgcgtgaccatgactcgcgacacctccatctccacagcctacatggagctctcccgcctgcgctctgac

SEQ ID NO: 40 - DNA, 104 bp, Artificial
  oligonucleotide
gacaccgccgtctattactgtgcacgctccactatgattaccaactatgttatggactactggggtcaaggcaccctggtcaccgtctcctcaggtgagtcctg

SEQ ID NO: 41 - DNA, 99 bp, Artificial
  oligonucleotide
tcgacaggactcacctgaggagacggtgaccagggtgccttgaccccagtagtccataacatagttggtaatcatagtggagcgtgcacagtaatagac

SEQ ID NO: 42 - DNA, 93 bp, Artificial
  oligonucleotide
ggcggtgtcgtcagagcgcaggcgggagagctccatgtaggctgtggagatggaggtgtcgcgagtcatggtcacgcggtccttgaatttctg

SEQ ID NO: 43 - DNA, 92 bp, Artificial
  oligonucleotide
gttgtagagagtaacaccattgttaggattaatacgtccaatccactcaaggccctgtccggggctctgcttcacccagtgcatgtagtagc

SEQ ID NO: 44 - DNA, 97 bp, Artificial
  oligonucleotide
cagtgaatgagtaaccagaagccttgcaggacaccttcactgaagccccaggcttcttcacctcggctccagactgcaccagctggacctgggagtg

SEQ ID NO: 45 - DNA, 90 bp, Artificial
  oligonucleotide
gttactcattcactggctactacatgcactgggtgcgccaggcccccggacagggccttgagtggatgggacgtattaatcctaacaatg

SEQ ID NO: 46 - DNA, 92 bp, Artificial
  oligonucleotide
gttgtagagagtaacaccattgttaggattaatacgtcccatccactcaaggccctgtccgggggcctggcgcacccagtgcatgtagtagc

SEQ ID NO: 47 - DNA, 101 bp, Artificial
  oligonucleotide
gtgttactctctacaaccagaaattcaaggaccgcgtgaccatcactcgcgacacctccacctccacagcctacatggagctctcctccctgcgctctgag

SEQ ID NO: 48 - DNA, 93 bp, Artificial
  PCR primer
ggcggtgtcctcagagcgcagggaggagagctccatgtaggctgtggaggtggaggtgtcgcgagtgatggtcacgcggtccttgaatttctg

SEQ ID NO: 49 - protein, 30 aa, Artificial
  consensus antibody heavy chain framework 1 sequence based on human antibody sequences
QVQLVQSGAEVKKPGASVKVSCKASGYTFT

SEQ ID NO: 50 - protein, 14 aa, Artificial
  consensus antibody heavy chain framework 2 sequence based on human antibody sequences
WVRQAPGQGLEWMG

SEQ ID NO: 51 - protein, 32 aa, Artificial
  consensus antibody heavy chain framework 3 sequence based on human antibody sequences
RVTITRDTSTSTAYMELSSLRSEDTAVYYCAR

SEQ ID NO: 52 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 53 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 54 - protein, 98 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 55 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 56 - protein, 98 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 57 - protein, 98 aa, Homo sapiens
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 58 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 59 - protein, 98 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 60 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 61 - protein, 98 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 62 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 63 - protein, 101 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 64 - protein, 98 aa, Homo sapiens; identical to SEQ ID NO: 52

SEQ ID NO: 65 - protein, 98 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWPPTV

SEQ ID NO: 66 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 67 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 68 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 69 - protein, 96 aa, Homo sapiens
EIVMTQSPPTLSLSPGERVTLSCRASQSVSSSYLTWYQQKPGQAPRLLIYGASTRATSIPARFSGSGSGTDFTLTISSLQPEDFAVYYCQQDHNLP

SEQ ID NO: 70 - protein, 96 aa, Homo sapiens
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 71 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 72 - protein, 95 aa, Homo sapiens; identical to SEQ ID NO: 71

SEQ ID NO: 73 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 74 - protein, 95 aa, Homo sapiens; identical to SEQ ID NO: 73

SEQ ID NO: 75 - protein, 95 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 76 - protein, 95 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 77 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 78 - protein, 95 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 79 - protein, 95 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 80 - protein, 95 aa, Homo sapiens
AIQMTQSPSSLSASVGDRVTITCRASQSISSWLAWYQQKPGKAPKLLIYDASSLESGVPSRFSGSGSGTDFTLTISSLQPEDFATYYCQQYNSYS

SEQ ID NO: 81 - DNA, 321 bp, Artificial
  artificial sequence is derived from mouse and human antibody sequences
gatattgtgatgacccagtcccccgactccctggccgtttcactgggagagagggccaccataaactgcaaggccagtcagagtgtgagtaatgatgtggcttggtaccaacagaagccagggcagtcccctaagctgctcatatcctatacatccagtcgctacgctggagtccctgatcgcttctccggcagtggatccgggaccgatttcactctgaccatcagctccttgcaggctgaagacgtggcagtttatttctgtcagcaagattataattctcctcccaccttcggtggaggcaccaagctggaaatcaaa

SEQ ID NO: 82 - DNA, 360 bp, Artificial
  artificial sequence is derived from mouse and human antibody sequences
caggtccagctggtgcagtctggagccgaggtgaagaagcctggggcttcagtgaaggtgtcctgcaaggcttctggttactcattcactggctactacatgcactgggtgaagcagagccccggacagggccttgagtggattggacgtattaatcctaacaatggtgttactctctacaaccagaaattcaaggaccgcgtgaccatgactcgcgacacctccatctccacagcctacatggagctctcccgcctgcgctctgacgacaccgccgtctattactgtgcacgctccactatgattaccaactatgttatggactactggggtcaaggcaccctggtcaccgtctcctca

SEQ ID NO: 83 - DNA, 360 bp, Artificial
  artificial sequence is derived from mouse and human antibody sequences
caggtccagctggtgcagtctggagccgaggtgaagaagcctggggcttcagtgaaggtgtcctgcaaggcttctggttactcattcactggctactacatgcactgggtgcgccaggcccccggacagggccttgagtggatgggacgtattaatcctaacaatggtgttactctctacaaccagaaattcaaggaccgcgtgaccatcactcgcgacacctccacctccacagcctacatggagctctcctccctgcgctctgaggacaccgccgtctattactgtgcacgctccactatgattaccaactatgttatggactactggggtcaaggcaccctggtcaccgtctcctca

SEQ ID NO: 84 - protein, 447 aa, Artificial
  Humanized heavy chain with variable region derived from human and mouse and human IgG4 constant region
QVQLVQSGAEVKKPGASVKVSCKASGYSFTGYYMHWVRQAPGQGLEWMGRINPNNGVTLYNQKFKDRVTITRDTSTSTAYMELSSLRSEDTAVYYCARSTMITNYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK